SEQUENCE LISTING (65 sequences)

SEQ ID NO: 1 - DNA, 930 bp, synthetic construct
  source 1..930: mol_type = other DNA
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaa

SEQ ID NO: 2 - DNA, 1051 bp, synthetic construct
  source 1..1051: mol_type = other DNA
aaatctgagacaacgatgaacctcccatgtagattccaccgccccagttacttttttgggcaatcctgttgataagatccattttagagttgtttcatgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaa

SEQ ID NO: 3 - DNA, 1046 bp, synthetic construct
  source 1..1046: mol_type = other DNA
gatctctgagacaacgatgaacctcccatgtagattccaccgccccaattactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaa

SEQ ID NO: 4 - DNA, 564 bp, synthetic construct
  source 1..564: mol_type = other DNA
tgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagacttctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaa

SEQ ID NO: 5 - DNA, 750 bp, synthetic construct
  source 1..750: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 6 - DNA, 1005 bp, synthetic construct
  source 1..1005: mol_type = other DNA
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacag

SEQ ID NO: 7 - DNA, 1002 bp, synthetic construct
  source 1..1002: mol_type = other DNA
cagccattaatctcacctcagtttttgaatcagtagaatttttaatgaaacaaacggttggtatattatttgatagagttgccaaatttccaaagataaatttttcatcaggtaatatcctgaataccgtaacatagtgactattggaagacactgctatcatattatatttcggataaaaatccaaaccccagaccgacctcttgagtctcaactccaagtcagccgcaactttaattatccgtggattgggagctagtttggacaacgcatcagtataatataactttacggttccattatcagacgctattgcaagaacttcctttccattgatctcgccaatgcggcagtaattgatatcgtagggtaggtctggaaagacgctggcgcttgtgtcccattctgcaggaatctctggcacggtgctaatggtagttatccaacggagctgaggtagtcgatatatctggatatgccgcctataggataaaaacaggagagggtgaaccttgcttatggctactagattgttcttgtactctgaattctcattatgggaaactaaactaatctcatctgtgtgttgcagtactattgaatcgttgtagtatctacctggagggcattccatgaattagtgagataacagagttgggtaactagagagaataatagacgtatgcatgattactacacaacggatgtcgcactctttccttagttaaaactatcatccaatcacaagatgcgggctggaaagacttgctcccgaaggataatcttctgcttctatctcccttcctcatatggtttcgcagggctcatgccccttcttccttcgaactgcccgatgaggaagtccttagcctatcaaagaattcgggaccatcatcgatttttagagccttacctgatcgcaatcaggatttcactactcatataaatacatcgctcaaagctccaactttgcttgttcatacaattcttgatattcacagg

SEQ ID NO: 8 - DNA, 619 bp, synthetic construct
  source 1..619: mol_type = other DNA
cttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaacc

SEQ ID NO: 9 - DNA, 750 bp, synthetic construct
  source 1..750: mol_type = other DNA
gtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaaggcatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttcta

SEQ ID NO: 10 - DNA, 996 bp, synthetic construct
  source 1..996: mol_type = other DNA
ggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtattgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 11 - DNA, 425 bp, synthetic construct
  source 1..425: mol_type = other DNA
gtcgaggaaagggtcgtttcggggagttaaatatttttggctatgtagcagacatgtttcgacgctggcgtcgcgtcgatcggaaaatattaccccaggaacaagcacttgcttgggttagccaccaccctgcgcaagcctttttgccggctctacacagggccaatgaaatctgggcggaatctgaaaccgatgaaacggacgacactggcaacaagctcactgcactattttttttttctagtgaaatagcctatcctcgtctcgctcccctcatacctgtaaaggggtgcaatttagcctcgttccagccattcacgggccactcaacaacacgtcggctaccatggggtgcttgggcaccaaaaggcctataaataggcccccatccgtctgctacacagtcatctctgtcttttcttccc

SEQ ID NO: 12 - DNA, 1050 bp, synthetic construct
  source 1..1050: mol_type = other DNA
aagatcgggttatccatgaaatcctttcaaggagtagagacctattcctcacgaaattcgcacatagctgcaagagttcgtggtgggtgcaacctcccaagaattacaaggctgaagatgggactgagatagaatgaacagcctcaaaaaagaaaataaaaccgtgagcctgagaacgaaagataagggtctagatgcgctcagacgtgaaacggcaacatcaattagttaaaatgaccgtaaaggcaattgatgtacgccgctgaagcaactgaatgaatagttccaaagaagtaggctcgtcaatttaaaatatctcgtagtcagttagtttactgatattcaggtgcttgacgcgcggtttgcacccatatcaaccaatcacacctcttgatcggatcaaaaagactccagaaatccagaaaatcaaatgctgtggagctggaatcctaaactccttcagttcatcctcttccagctctgcctgcctcaacttagatcagggctgataaataattttcataccactaataaagtcacgtgaaattggtattaataacccctcctctgtcatagtgcattgcttatcgataatagggggttcgcgcaaaagaacagggctgtggccgcgggtcccttccgaccccctttagttgtagagagcagcaaagcactagttgacagatttaaagcttcgccgcttcacgtggccttcgaggttgccacatctgcaaaagttcacctatcaaggtttcagatcggttatcagtaacttagttggttcccctccatgaatcgaaatcaaaacacactcctgaagcgcaaccttctgattggcggattcaagcgagggtcacgatttttttttgaaattagcttgaaaaagagtcaaagaggggtgtgactgggccaattttttcggatgccaccccctcttttctccccatgacatctaaagaccgattgaaaaaaaaaaaaatcgccagggtataaattgtgtgaaattccctgatttaagtccaattactcttcatatcagtctcattaata

SEQ ID NO: 13 - DNA, 700 bp, synthetic construct
  source 1..700: mol_type = other DNA
ccggaggttggagattttgtatatataaaactttcttggagcttattaataaatgcgggatgcagtaaacttgcatatatctattgtaacacttttgcaatagctgcatgccttgactcatcattcagtatcgtgtgaaaaccaatgatacatccgtacattcaaactacaaccttcctcattagtaattctttttgaatttttcggaacccgaagctccgcctatccccccaactaacacatcttccaatttgggtgggagaacacctagcaacatcacgatcattgcgcgaacgttcgcactgtatttttttctcccaaacacccaacttctaggccaaatatccacttctcggggttctattcacccatttaattgttggccttaaaagtcaattgagttccaatcatagtccctagttgattgcttgtagcaaatgccacaacagtaggcatttacgtcctcacagtctcttcccttgtccctcattgatacctctttattctcccccaccaccatacactaccttcctcgcacccctgtcatcacaaccgcaatataatcgatgcgcggtttcttgcctaatccatcgtccaacagagaggtcgctctccttatatatatagttgatccccctttttttctacccttgcaattttttttttgggaccaaagaaaagaaacaagactgatacaaat

SEQ ID NO: 14 - DNA, 325 bp, synthetic construct
  source 1..325: mol_type = other DNA
gtacgttaagtatgccaaaaacatcacaatagacaatacgccctacaacagaacgtcaagttgtacgcgagcaccgttaaatcacacgtaacatccaacacctttctttggtagggcatagccccaggtggcaccacgtgcataaaccattttacaccccaacacccaccaatcctcgccctctggcatttgctcaaattttttaaccagcttctgattacataggtaaccagttcaattcataggtaagtcaacgccgaacaacaagggaaatcacccatccgctcacatcagtccagttgaagactaacattaaaagacaaaa

SEQ ID NO: 15 - DNA, 471 bp, synthetic construct
  source 1..471: mol_type = other DNA
gtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaag

SEQ ID NO: 16 - DNA, 680 bp, synthetic construct
  source 1..680: mol_type = other DNA
atttaaatgtaagatctttatataaatgaatctacatggtgtgttttatttagatcctccaaaccaaggaaagaaactaaacttatctccggacttacgagtcaaataactatccgcagttccttggaactcagactttcttccataagcggtcatatcatctttggactgtgggaatcctggacgaatctttgaaatgtcataatcttgctctctatctccaagcacagcgtccggtaaatgctggttcttctttctcagatgaatcttggatttaacaaataaagccgtgcctatggctaatgtactcaaaaacaaagtctgcttccagaatttcgcaaacgatggaatgccatttcctgtaaatgtactcattgaacctatgtttgattaaagttggtgtgaagtcatcaaacgagagtaaaatcagatactcgtgcaccggccaaaattgactgagctaatctctgctggcttgacatccgaacacaacaaataggcgacaaatcttaactatctaatcgtaggctatggtagaactttgtgggggtagaggaagactacaacagcaagacaaaacaaaagagtcatagtttgactctctgcttttttcttctttctcttctttttcttcctccatattcgttatttatttcgaactggacaactaattattgaaa

SEQ ID NO: 17 - DNA, 1501 bp, synthetic construct
  source 1..1501: mol_type = other DNA
tccagtgtagcactaaaatctaatatcttcggctttatacttttttgttcatccgaaagcttacgaacaattctttctcctgttttattgtggatatagacaatttcgtcagtttcttggagagaagagttatttccggttttggctggccctataaacgggttcttggatttggatctagtaataaaaatgtcactgtcattctcggagctgaactttgtgttgtacgaagatgggttgttccactgttttgccagctcttcattgatgattttcttagtgggtgttcttggaggttcacgttgcctataatcttgacgttcttcttcatcactatcgatgccatcaaaattaagcgtccttattgcaggcttttgtgatttcaactgcaatccttctatctcttcatcagagctttcgaactgaatactatcactcaaaactggcgacattgcacatttccgcaaaccatttcgggaatctatgctagctcttctagacgataaagaacgaccggaaccaatacggggttgtgcaggtgggaataaatatgttggtttggattcttgacgtgaagaaggtattctagtcgatgaagtggttgataaggatatggcgtcactgagttgttttcttttcctatgttgcggtgttgggtcaggagttaattgattcacctccataactctggaatttcttgaatgtggggttttcagatgggcatctttcttgacggggttgtgagtaacggaggaacctggtgtcttgggtgtgaacggtgtttgagcctgtacgcggttacttctgggcggagtactcggagtcatgagagccattgattagaaggtgaatgagggagtcaccactctaagcaaacaaaatgaggtcgaagcaaaaaataaagtaaagtagcacttctggcaggttagatcaaagagtgacgggagatttgaagatggctggtttttccttagtcttggaagaggtttgtgtgggtatcagcgaatattccccgattaggcaaattagttgcattgaaattaacacgacatggtgatttgtggtaacaaatatctattggtggttggtgtgtgggtgtaatagtggtcgtgtcatgatgatggtgttcaggtgttgtcatagatcggtcttcagtaagagaaggaagcttggtgacgatcacagctatgatgtaatagaaattgctaagcaattgtgaggtgtgatgtattttgcagagcaattgtgcggtacaacggggtgttattgtcttcacaaggcatttattgcgaatttcgtagttgaaagaatattttagcacagggtgcttgacccctattgttgctcgctaaaccatgattgctaaatgatgacatagcaatcactttactaagattgctataaggacacctttcttagtataaatggacactcttttcccctgctaaacttcttttatttttcacacttaaacagttacaaaacacaaacacaactagaa

SEQ ID NO: 18 - DNA, 1501 bp, synthetic construct
  source 1..1501: mol_type = other DNA
gtgctaaaatctgaggtttacaagctgtgatgttcccctaagatctcacaatcgaacaatcgcgaagccaatgcaagttgtttaaggggaaacgactcactattcctgaaattagtattcaaaacttggtccggaagaacaatgaggcggccgttaaaatactcacgtaaacggtgtctacaagcgcattaaaatccgtttgaattcaagcaaaagccaccagaggcttatgcttggttatacccagcattgacctttggtatgagcatctgaaaaacaaccaggtgttgcaaagttaaacatccttctttgttcatatagaacccactattcatggtactccccaatcgaatttcacattctggttttgaaattacacaccacgttagcttataagatttcatataacttattgatatacggtttccattgttcgaatagttgaggttgtatgtaattcgattgaaggggccatttttgtttcctacttttcctgggagcttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 19 - DNA, 1500 bp, synthetic construct
  source 1..1500: mol_type = other DNA
tcacattcatagcatctctcgcctgcaatagcttccacgataggaatatctgtgaaagtgaacatgctatttcgatgatataagactttaagatctggcatgtttgtgttggaggttaccctggggtcaataaccctaattatctccttcactaaaaatgatgaagattcttcggattcgtttttgaacagagttaatgccatttcttcgtcaatagaaaaatcaatatctggtatctcatcttttacatattgaggatttagttttcttccctttggatagtacattatgatcaatgtattcctgtctttattgataaagtattggcattctgcttcttgtacacctttgaattgtttgtctggaagtgactgacatttttccacattgctaacggtttggcacgaattacatctaaataaaatgtcttctccggattcgtgtattaagtgatactccaatgataaatccccacctatcgaaccagaatcggcattggccacagtcacaggtaactttaggtcttgaaaaatccttctataggcttcattgacattgtcataagacttaagaccatcttctttggtcaagtcaaaagaataggcatctttcatgagaaactctcgtcctctcaacaaacctcccctaggtctcaactcatctctatatttgcgggaaatttggtacacgagaaggggtaaatctttatatgacgaacataagtcaccaactaagtttgtgatttcctcttcacaagttggcactaaacagtagtctctatccttggagtctttgaacttgaacaattcattgttgtcccatctcttagttctctcccataaatgcttggaagacaggctacttaattccatttccagcccaccagcctgatccattcttttcctaattacattttgaagctttttataggtacggagtcctaatggaagccagtgaactattcctgctgcaggctggtaaataaaccttgattgaaggagcatatcatgagtagtaaggtcctttacagaaaatagtttacttccttgaagagaagtagaataaaacctcatgttgggtctccatgaaaggttcaaaggcattgatcctttaggtacttcaggatgtttaagtcatcaaactgtccatcaaaggtagtatagtatttaccatctagatagtgatgtatgggtgtaacacaacatttaaatgttgtaaattaacattaggactgagtccggagatgctattgtcacctaaatctattagaaagcacttcagttatatcatcgatagaggtttgaagataaacctattgttgataaataaccccattacccgtttacgtagcaaggttcaaaaatttgcttagatcggagctaaaaattcgactgacttctttcgaaaatgtggattatgcaagcaacgttgctatcggaatagtatataaggtcgatctgccccattacaaattgtaaagcaacaaacatcctacgcaaa

SEQ ID NO: 20 - DNA, 1500 bp, synthetic construct
  source 1..1500: mol_type = other DNA
tcagtttcacggttatgtgagctgtctccgcgtgaggcagtaacctctgtgtcatggatacaggctggtacacatttggcagtaggaacacaatctggtttagttgaaatatgggacgccacgacgtccaaatgtacaagatcaatgactgggcattcggcccgaacctcagcgctgagttggaaccgtcatgttttgagttctggttcaagagatcgcagtatcttacatcgggatgtacgtgcagcagctcactatacaagtcgcattgttgaacaccgccaagaggtttgtggcttacgttggaacgtggatgaaaacaagctggccagtggttccaatgataaccgtatgatggtatgggatgcactgcgtgtagaacagccccttatgaaagttgaagagcatactgcggctgttaaggcgttggcatggtcacctcatcaacgtggaatactggcttcgggtggaggtactgctgacagacgtatcaaggtgtggaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacctgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 21 - DNA, 1500 bp, synthetic construct
  source 1..1500: mol_type = other DNA
agaagtactgttatgaatcgatcgacgtgacatgttgttgatggttctgacttcttgatgtccgcgttttctgtctctcaatagtggtgttcgggggaagtatggttctaatacttaacaggtaagatggttgcaatgagcacctggtaaagcaacttgaatttcctgccctgtctccgttaagttatattcgactcaaggtccttgcttcctgtctgttctgtaaaacttccctttggtgtcttctatatcaactttaaaaacaaggtagtgtgtcgagcgatagtactgtgtctttttccctatgaaaaaaatcgcaccatccaagacttctcaccttcaacagcttcaacatcatgttcggtccttttagagctacgctggtcgatctaggaggtctgctatggaaacgtccttggagaatgtccaaaccacagaaatatagactccgcaaaagaatgcaacttgtagactccaatatcgacattatttaccagggactgactgaggagggtctgtcttgcaaagtgatagataacttgaaacaaaacttcccaaaggagcatgaagtgctccccaaaaacaagtataccgtgtttaacaagacagccaaaaactatagaaagggtgttcatttggttccaaaatggaccaagaagtctttgagagagaaccccgagttcttctaattgcacatttcttcctgttcatagattatcccacacatagttgctcacaaaaaaatcactataattttcctccaccggcagtatatcactaacacctttatctttattgtagattataatctgatctttatccttagatgtatctatcatcaaccccatgctcttgaaaagcttgagtcttaacactgtcgaatcgtagttttcttgtagatcattcgatatcactgctttttcttgctcttctaattcgttgagattctgggtcaaactagagattgaattctgaaggtgattcatgttcatctccagatctgttattgattttgctaatttaaatttttcgtgttcaagctcttcgatactctttagggtctgttgacggtcttctgtttccaataattgcttgttgaactctttaagttcgtctctctgtttactgatacgtgacaacaaatctagctggtgatcgagtttaagtttccgtttggagctcaacagagaaagattttcattaatttggttgatagtttgcacgtccggttcgatctgaaaattctctatagtcgacctgattaaggacacagtctcttgaagatcggacattggatttatggagaagggagatcaaagcggaaccagttgcactgtttacctttccagtcgagatacttatcccacagggccctcactttccaggcagaagtcacctaggaggcgcatccctccgtttgcttccctcgcgacaaactcccctgtaaaagaaaacttcactgaatcgtacacctaatcatacgacactaacacagatata

SEQ ID NO: 22 - DNA, 1500 bp, synthetic construct
  source 1..1500: mol_type = other DNA
gtcctttccaaatttttggttgaaggcatcgcttaaattatgagcaggatcggtggaaataagcaggtatttcttgttaggattgtgaagggcaagctggatagatatagaagaagatgtcgtggttttaccgacaccccccttacctccaacaaagatccacttcagcgattcgtggttcacaattgatcgcaaacttggctctgcctcaatatccatggttgatgtctagttgagtggcgtttgtggtctcttgatgagttcaaggcgaaagaatatgataggaaagcatggtttgaacttttcgcgaaagaaggaatactgttccgcgagaaactccccggtgccagaaccttccattgaggttaatcggtgggaggtgttcgaatgacaatgtcagacaaggcgaacacgtcttgtgacaccagctggactaagaagattcggtatgcaccgaagaagaaggccgtgtctcaattggcaactttgcaacaaactacggaggaaaagtctcacaagcttttaaccaagttgaatcacgacgacaacgataaagaaatcctcaaccatctaacacatgaagtacaaagtagaaatgtgatcttattggacaaactagaggagctcaacaaggaactgggctggattaaagaccgaaaatgaggaaccatgagcactgggcgtttccagaaaaactgcaaccaacgatgggaaaatgataccacactactatggtcaccccacattgtgaaatttcaaaccaaaaaagatcaaccccataattccccagagggttttcccaacaattttccaacggacttgataatgagtcagatcatttgagcatattcatcttaccccttattccgtgacaatttacctattccattcaaagcatacggtatcccgtgaccttctcatggagatcattctccaccgatacagcatatacacagatatacccaactaatatcaattggaccttgatatggtcgaccttgatggtcccgtccaaccttaaaacttagtttaatgctatactttcgccttgaaccaaatctgtctccccctcaatcatctctatgcaagaaggtcaacactgattacgtgagcaacagccagcaatcgttcgagtccccgccaaaaaaggcggagttactgctccttgtgaccacaccccctgagaccacgtccctaaacgatccttgtcggttccttcgtccaattggcaattgccacgcatacgtgaatcgttattgtttcgcctaccttgcgtcattcgttccagaatgttcgacatactcctctagaacataccgtcacaccaccatcttaagttatcttcacgtgaccatgacgtacattgtagttgactaccccattctcatcattccgatgcggccaaaaatctctatataaagaccgtatcccctaatattctcttcttgttaagacattaacttagttaattcaccaattactcacttataaacaaacaaa

SEQ ID NO: 23 - DNA, 1500 bp, synthetic construct
  source 1..1500: mol_type = other DNA
gtttctcttggggagatacttttttcgcgtgctcctccgtgcggaacttccttctgagcttctacctctcagattagtctaatcgcatcaggaataagactgagaatgcttttaaggagaggcttgagattggctaattgcgttccgaagtactctttcaaaaggagttatacccctctcaactacgattctctaaagaattatcgtaggcatgctcaggcgcctcaaccccatcagtttgacgccactagatgggaccaacaaccagttactaatgagcaaggagtaatactcccatccgactcaattgcaaacattctgagacaaccaactctggtcatagaacggcaaatggaaatgatgaatatatttttaggatttgagcaggcgaaccgatatgttatcatggatcctacaggaagtattttgggttacatgctagaaagggatctgggcatcaccaaagctatattgagacagatctaccgtttgcatcgaccttttacagtggatgtaatggatactgcaggaaatgtattaatgacaatcaagaggccgtttagtttcatcaattcgcacatcaaagctatattaccccctttcaggaacagcgacccagacgaacatgtaattggagaatccgttcaaagctggcatccttggagacgaagatacaatctatttacagcacaaattggcgaaaaggacactgtctacgatcagttcgggtacattgacgcaccgtttctttcctttgagtttcctgtactttcagaatctaggcaaacgctaggtgctgtctctagaaacttcgtgggctttgcaagagagcttttcacagatacaggagtttacatcatccgtatggggcctgaatcttttgtagggctagaagggaactacgggaacaatgtggcccaacatgcccttacgctggaccaaagggctgtattattagccaatgccgtttcaattgactttgattacttttctaggcactcgtcacacagtggtggcttcattgggtttgaggaatagacagggtctcgtcaactcagctcctgccaccaaaccaatcattgatcaacgagcacacttttgtccacgtgagatcgctttcgcttgcagaaagagcaatgcatgaaaacggcaaacgcaaaacgagcaaaaaaacgagtaaataactacaatttcaccaccaacagggtcaaagagcttttgagacactataaaaggggccctttccccccaggttccttgaaatcctcattcaattatgttttttactcataatttgactcaattggcatcttcttctttgttcatatacagtaattgatatgacgcttagtcattattagtgttctcgactagcagtggcgaaaaaagggggagttattttctagaaccgaccgcaaactataaaagaaagctgcccctcatatacctttcgaattctttattttctgtgtttcttccctatttaacatctacacaaaa

SEQ ID NO: 24 - DNA, 261 bp, synthetic construct
  source 1..261: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcg

SEQ ID NO: 25 - DNA, 476 bp, synthetic construct
  source 1..476: mol_type = other DNA
aattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgca

SEQ ID NO: 26 - DNA, 499 bp, synthetic construct
  source 1..499: mol_type = other DNA
acgggaagtctttacagttttagttaggagcccttatatatgacagtaatgctagtacgttttgttttgtttaattaataacttagtttatgttagcctagtatagactccatcaattttttttgttattacgtaagccgcgatgataatatctgatgaaaaattcctatcagaaaataatttatcaaaagtttcatgcgatatgagactaagtagaatagggactcccaaagtgtcagtcacaagggtcattcccgttcgtaatgtggtgatagcgaggagaaaacctgtcagagcaagtaacaccgacgcaaagacatggctaatgaaagaagagcagagaagaataagacagaaggagcaggagatgaaacaaaggctagaggaactagaaaggttcaaaacaaaagtacagaaatcatatataaggaaagaggataggcatttggcacaagagatagaaaaggatcttgacataatcactgatgattacaatttg

SEQ ID NO: 27 - DNA, 194 bp, synthetic construct
  source 1..194: mol_type = other DNA
ggagacgtggaaggacataccgcttttgagaagcgtgtttgaaaatagttctttttctggtttatatcgtttatgaagtgatgagatgaaaagctgaaatagcgagtataggaaaatttaatgaaaattaaattaaatattttcttaggctattagtcaccttcaaaatgccggccgcttctaagaacgttgtc

SEQ ID NO: 28 - DNA, 202 bp, synthetic construct
  source 1..202: mol_type = other DNA
tcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 29 - DNA, 267 bp, synthetic construct
  source 1..267: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagct

SEQ ID NO: 30 - DNA, 267 bp, synthetic construct
  source 1..267: mol_type = other DNA
atgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaagctgaagct

SEQ ID NO: 31 - DNA, 561 bp, synthetic construct
  source 1..561: mol_type = other DNA
gctgaagtagactgctcaagatttccaaatgctactgacaaggaaggaaaggatgtcctcgtatgtaacaaggaccttagacccatttgcggtacggatggcgtgacatacactaatgattgtttactatgtgcctatagcattgagttcggtacaaacatctccaaagagcacgatggagaatgtaaagagactgtccctatgaactgttcctcttacgcaaatacaacttcagaggacggtaaggtgatggtcttgtgtaacagggctttcaatccagtttgtggtactgacggtgttacttacgataacgaatgtctgttgtgtgctcataaagttgagcaaggagcatctgttgataaaagacacgatggtggatgccgtaaggaattggccgcagtttcggtggactgctccgaatatccaaaacctgactgtaccgctgaggatcgtcctctgtgcggaagtgacaacaagacctatggtaataagtgtaatttctgtaatgctgttgttgaaagcaatggtacattaacattgtctcattttggtaaatgttaa

SEQ ID NO: 32 - DNA, 558 bp, synthetic construct
  source 1..558: mol_type = other DNA
gcagaagttgactgttctcgtttcccaaatgctactgacaaggaaggaaaagacgtcttggtgtgtaacaaggatttgaggccaatttgtggtacagatggtgtgacttacactaatgattgtctactttgcgcatatagcatcgagtttggaaccaatatctcaaaagagcacgacggtgaatgtaaagagactgtcccaatgaactgttcttcctacgctaatacaacctccgaggatggtaaagtaatggttttgtgcaacagagcctttaatcctgtttgtggcacggatggagtcacttatgataatgaatgtctcctgtgcgcccacaaggtagaacaaggtgctagcgttgataagcgtcatgacggtggatgtagaaaggaattagctgctgtgtctgttgattgttcagaatatcccaagcctgactgtacagctgaggacagacctctgtgcggttccgacaacaaaacatacggaaacaaatgcaacttctgtaatgcagtggttgagtcgaatggaacattgactttaagtcatttcggtaaatgt

SEQ ID NO: 33 - DNA, 1113 bp, synthetic construct
  source 1..1113: mol_type = other DNA
ctagtaaaggtgcctctagttagaaagaagagtctgagacaaaacctaattaagaacggaaaactgaaggatttcttaaaaacgcataaacataaccccgcctccaaatactttcctgaagcagccgctttaataggcgacgaacctttagaaaattacttagataccgagtatttcggcactattggtattggtacgcccgcacaagatttcacggtaatcttcgacaccggcagttcaaatttatgggtgccctccgtgtattgtagtagtttggcttgctccgaccataatcagttcaaccccgatgattcctccacgttcgaggccacgagtcaagaattgagtataacctacggcaccggttccatgacaggcatcctaggatacgatacagtacaagtcggcggcatttccgacaccaatcagatatttggcctaagtgagaccgagcccggatctttcttgtactacgcccctttcgacggaatcttgggtctagcttatcctagtatatctgcatccggagctacacccgtgtttgacaacctatgggatcagggccttgtctcccaggatctattctcagtctacctgagtagtaatgatgattcaggctcagtagtgttgctaggcggaattgattctagttactacacaggttctctgaactgggttcctgtcagtgtagagggctattggcagatcacactggattccataactatggatggagagaccatcgcctgctccggcggttgtcaggcaatagtggataccggaaccagtctgttgactggccctacctctgccatagctaatatacaaagtgatataggagcatctgagaactctgacggcgagatggtaatctcttgttctagtatcgattcattacctgacatagtttttaccataaatggtgttcaataccccctaagtccttccgcctatatcttgcaagatgatgactcatgtacaagtggctttgaaggtatggatgtacccacgtcatcaggtgagctttggatactgggcgatgtgtttatcaggcaatactacaccgtgttcgatagggctaacaacaaggtgggtctagcacctgttgcataa

SEQ ID NO: 34 - DNA, 1885 bp, synthetic construct
  source 1..1885: mol_type = other DNA
agtgaaaacgaaaagtgaaaatatcctgaggacgacttttattcttttggctggtgctagcgctgcatgttcgttactagccgttcaatacccattttctaaagttcagtcaatacatttagcaaggttggaagcgttggatattttcaacgagaactcctgggataagaaaagtaaattccgtctatattaccgatcatacttagatacattccaccagttggtgagcttacatgagaagtctaaactatcttggacccgctggttttataaagggttcgttaggaatgcgttaactaccattccagcaacatccgtggggcttctggtgtttgaaatactgcgtcaaaaattgagcgatgaaattgaagatcgattcagttgaatcgcccgaaacaattgatcccctgtacatacttgtaatttacctcagaatattttggtaagcttcccacccagcttttctataccgttcaccttcttttaagggatctctgcccttgccaaacaaaccacgacctacgattatgatgtcagtgccagtggaaaatacttgactcactgttcgatattgttggcctagagcatcaccagtgtcatccaaaccaacacctggtgtcataataatccaatcgaacccttcatcttgtcctcccatagaattttgagcaataaacccaatgacgaattccttgtctgattttgcaatttctacagtttcttcggtgtacttaccatgggcaattgatccctttgacgacagttcagccaacatcaatagtccccttggttgatctgttgtctcagtggctgcctcctttagaccctttacaattccactaccaatgacaccatgagcatttgtaatatctgcccattgtgcaatcttgtagacacctccttgatattgatgcttgacagtgttgcctatatcagcaaactttctgtcctcaaaaattaaaaacttgtgtttctttgatagttccaataaaggcagaatagttccatcatacgtgaagtcatcaattatgtcgatatgagtcttggccaaacagataaatgggcccaatttatctagaagctccaataattctttagttgttctcacgtcgactgatgcgcataggttactctgtttctgttccataagcgcaaacagtcgtcgtgccacaggtgattgatgagtatttgctctctcggcataactgcgagccattgtctaggtatctatccctttgatcaggttgatgttaactcattagagtggatcaatgcgaaggataggtgcgacgtgtaccgtccaaaaaacttttttcttcaatcttgacaaaaactggtaacagagagagcaagtgctaactctaccccaaccaagtacatcacaaaatggacgcattgaacgctaaagaacaacaagagttccagaaactcgttgaacaaaaacaaatgaaagacttcatgcgtctttactccgatttggttagcaaatgttttacagactgtgtcaatgattttacatctaacaagttgacttctaaggaggaaggctgcatcaacaagtgtgcagaaaagttcctcaagcacagtgagagagttggtcaacgtttccaagaacaaaaccaacttatgatgcaacagctaagacgttaaccccatatttttgtacataaagttcattgtccaggactaatccagactttctctgaacagctctataatcttagtagtttcttccatcatttcaatcgttagcttcgaaacatcactgtcttcatcgttatagatgatggcatttgtaaacatgatctgtagaactttagtcaattcgtcaaaggtggttatctccccatttctgcagtgcttgagaatggtcttcagatcttga

SEQ ID NO: 35 - DNA, 375 bp, synthetic construct
  source 1..375: mol_type = other DNA
atggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaa

SEQ ID NO: 36 - DNA, 810 bp, synthetic construct
  source 1..810: mol_type = other DNA
atgggtaaagagaaaacgcacgtcagtcgtccaagattgaactccaatatggatgcagacctgtacggttacaaatgggctagagataacgttggacaatctggtgcaactatatatagattgtatgggaagccagacgcaccagagttgtttctaaagcatgggaaaggctctgttgctaatgatgtgactgatgaaatggtacgtttgaattggctaacagagtttatgcccttgcctactattaagcattttattcgtactcccgatgacgcttggttgctaaccaccgcaattcctggtaaaactgcctttcaagttctggaagaatacccagattccggtgaaaacatcgttgacgccttggctgttttcctgcgaagacttcactctattcccgtatgtaattgtccctttaattcagacagagtttttagattggctcaggctcaatctaggatgaataatggtttggttgatgcaagtgacttcgatgacgaaagaaacggttggcctgtcgagcaggtgtggaaggaaatgcataagttacttccattttctcctgattctgttgtaacccacggtgatttttccctagacaaccttatattcgatgagggcaagttgattggttgtattgacgtcggcagagtgggtatcgccgataggtatcaagatttagcaatactgtggaattgtctaggagaattttcacccagtctgcaaaagagattgttccagaaatacggaattgacaaccccgatatgaataagttgcagtttcatttgatgttggacgagttcttctaa

SEQ ID NO: 37 - DNA, 1029 bp, synthetic construct
  source 1..1029: mol_type = other DNA
atgggaaagaaaccagagctgaccgcaacgagtgtcgaaaaatttcttattgaaaaatttgatagtgtgtccgatttaatgcagcttagtgaaggcgaagagtcacgtgctttctcattcgacgttggtggacgtggctacgttttgagagttaatagttgtgcagatggcttttataaggatcgttatgtataccgtcattttgctagtgcagccctgccaatcccagaggttttagatataggtgagtttagtgagtctcttacttattgtattagtcgtagagcccaaggtgttacccttcaggatttgccagagactgagcttcctgctgtattgcaacctgtcgctgaggctatggacgccattgccgcagcagatttatctcaaacgtcaggtttcggccccttcggcccacaaggcatcggacagtacacaacgtggcgtgactttatctgtgccatcgctgaccctcatgtctaccactggcaaacggtcatggatgacacggtgtccgcctctgtggcccaagcattggatgaactgatgctttgggctgaggattgtcccgaagtccgtcacctggttcacgctgacttcggctccaacaatgttttgaccgacaatggccgtatcaccgctgtcatcgactggtctgaggcaatgtttggcgactctcagtatgaagtcgccaatatatttttttggagaccctggttggcatgcatggaacagcaaactcgttactttgaaagacgtcatccagagttagctggtagtccacgtctgcgtgcttacatgttgcgtatcggcttagaccaactgtatcagtcacttgtcgatggtaactttgatgacgcagcatgggcacaaggacgttgtgacgctattgtacgttcaggtgcaggcacggtcggccgtacacaaattgcacgtagaagtgcagcagtctggaccgatggttgtgttgaggtccttgcagattcaggaaatagacgtccatctactcgtcctcgtgctaaggaataa

SEQ ID NO: 38 - DNA, 25 bp, synthetic construct
  source 1..25: mol_type = other DNA
ggaattgtgagcggataacaattcc

SEQ ID NO: 39 - DNA, 1032 bp, synthetic construct
  source 1..1032: mol_type = other DNA
atggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcgcatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaa

SEQ ID NO: 40 - DNA, 34 bp, synthetic construct
  source 1..34: mol_type = other DNA
ataacttcgtataatgtatgctatacgaacggta

SEQ ID NO: 41 - DNA, 34 bp, synthetic construct
  source 1..34: mol_type = other DNA
taccgttcgtataatgtatgctatacgaagttat

SEQ ID NO: 42 - DNA, 673 bp, synthetic construct
  source 1..673: mol_type = other DNA
cccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcaca

SEQ ID NO: 43 - DNA, 110 bp, synthetic construct
  source 1..110: mol_type = other DNA
gttacaaataaaaagtatcaaaaatgaagcctgcatctctcaggcaaatggcattctgacatcctcttgacctcgaggttatcaggcttcagcttctctcttgtccaaag

SEQ ID NO: 44 - DNA, 61 bp, synthetic construct
  source 1..61: mol_type = other DNA
aaacacttcttccttagcagcaatagaagcaatagtagtgttaatgaacaacaaaccgttg

SEQ ID NO: 45 - DNA, 25 bp, synthetic construct
  source 1..25: mol_type = other DNA
acagcaacgtcgaagtcaccttcca

SEQ ID NO: 46 - DNA, 274 bp, synthetic construct
  source 1..274: mol_type = other DNA
cgaaacgatgagattcccttctattttcactgctgttttgttcgctgcttcttctgctttggctgctccagttaacactactaccgaagacgaaactgctcaaattcctgctgaagctgttattggttactctgacttggaaggtgacttcgacgttgctgttttgccattctctaactctactaacaacggtttgttgttcattaacactactattgcttctattgctgctaaggaagaaggtgtttctttggacaagagagaagctgaagcc

SEQ ID NO: 47 - DNA, 274 bp, synthetic construct
  source 1..274: mol_type = other DNA
cgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaaaagagaggccgaagct

SEQ ID NO: 48 - DNA, 97 bp, synthetic construct
  source 1..97: mol_type = other DNA
attgccattcgcctgggatatactttcccccagtttatgtatggctcaaaggcttacgttaaacatccagccgatatacctgattatttaaaactgt

SEQ ID NO: 49 - protein, 20 aa, synthetic construct
HCHSPGIYFPPVYVWLKGLR

SEQ ID NO: 50 - protein, 6 aa, synthetic construct
TSSRYT

SEQ ID NO: 51 - protein, 5 aa, synthetic construct
LFKTV

SEQ ID NO: 52 - protein, 32 aa, synthetic construct
IAIRLGYTFPQFMYGSKAYVKHPADIPDYLKL

SEQ ID NO: 53 - protein, 29 aa, synthetic construct
LPFAWDILSPSLCMAQRLTLNIQPIYLII

SEQ ID NO: 54 - DNA, 96 bp, synthetic construct
  source 1..96: mol_type = other DNA
attgccattcgcctgggatatactttccccccagtttatgtatggctcaaaggcttacgttaaacatccagccgatatacctgattatttaaaact

SEQ ID NO: 55 - protein, 30 aa, synthetic construct
HCHSPGIYFPPSLCMAQRLTLNIQPIYLII

SEQ ID NO: 56 - protein, 20 aa, synthetic construct
IAIRLGYTFPPVYVWLKGLR

SEQ ID NO: 57 - protein, 4 aa, synthetic construct
LFKT

SEQ ID NO: 58 - protein, 32 aa, synthetic construct
LPFAWDILSPQFMYGSKAYVKHPADIPDYLKL

SEQ ID NO: 59 - DNA, 93 bp, synthetic construct
  source 1..93: mol_type = other DNA
ggatatactttcccccagtttatgtatggctcaaaggcttacgttaaacatccagccgatatacctgattatttaaaactgtcattccctgaa

SEQ ID NO: 60 - protein, 24 aa, synthetic construct
DILSPSLCMAQRLTLNIQPIYLII

SEQ ID NO: 61 - protein, 6 aa, synthetic construct
NCHSLK

SEQ ID NO: 62 - protein, 15 aa, synthetic construct
GIYFPPVYVWLKGLR

SEQ ID NO: 63 - protein, 7 aa, synthetic construct
LFKTVIP

SEQ ID NO: 64 - protein, 31 aa, synthetic construct
GYTFPQFMYGSKAYVKHPADIPDYLKLSFPE

SEQ ID NO: 65 - DNA, 94 bp, synthetic construct
  source 1..94: mol_type = other DNA
ggatatactttccccccagtttatgtatggctcaaaggcttacgttaaacatccagccgatatacctgattatttaaaactgtcattccctgaa

(19) United States
(12) Patent Application Publication
(10) Pub. No.: US 2024/0209383 A1
(43) Pub. Date: Jun. 27, 2024

(54) SYSTEMS AND METHODS FOR HIGH YIELDING RECOMBINANT MICROORGANISMS AND USES THEREOF

(71) Applicant: Clara Foods Co., Daly City, CA (US)

(72) Inventors: Frank Douglas IVEY, Daly City, CA (US); Tyler DOUGHTY, Daly City, CA (US); Yuka SCHWARZ, Daly City, CA (US); Madeline TRAN, Daly City, CA (US); Lauren KOLYER, Daly City, CA (US); Ranjan PATNAIK, Daly City, CA (US); Joel Andrew KREPS, Daly City, CA (US); Charles Albert TINDELL, Daly City, CA (US); Weixi ZHONG, Daly City, CA (US)

(73) Assignee: Clara Foods Co., Daly City, CA (US)

(21) Appl. No.: 18/391,447

(22) Filed: Dec. 20, 2023

Related U.S. Application Data
(60) Provisional application No. 63/434,249, filed on Dec. 21, 2022.

(51) Int. Cl. C12N 15/81

(52) U.S. Cl. CPC C12N 15/815; C12N 2830/002

(57) ABSTRACT

Provided are systems and methods for production of recombinant proteins in engineered microorganisms. The systems and methods provide high-titer expression of recombinant proteins in large scale production and are particularly useful for expressing heterologous proteins in a microbial host, such as food-based proteins or other protein types such as therapeutic proteins and enzymes. Disclosed are also kits for making the same.

### CROSS-REFERENCE

This application claims benefit of and priority to U.S. Provisional Application No. 63/434,249, filed Dec. 21, 2022, the disclosure of which is incorporated herein by reference in its entirety.

### SEQUENCE LISTING

The instant application contains a Sequence Listing which has been submitted electronically in XML format and is hereby incorporated by reference in its entirety. Said XML copy, created on Dec. 19, 2023, is named 57854WO_CRF_sequencelisting.xml, and is 91,323 bytes in size.

### BACKGROUND

In industrial protein production, a goal towards cost reduction is to maximize expression of the protein product in the recombinant organism. Methylotrophic yeasts such as Pichia sp. are an important production system for proteins. Despite their widespread use, high yield expression, particularly for expression of heterologous proteins remains a challenge. This hurdle is particularly apparent in larger scale fermentation settings. While increasing the number of integrated copies can lead to increases in protein expression, there appear to be limitations to the amount of transcript produced with increasing copy number (Aw and Polizzi; Microb Cell Fact. 2013; 12: 128).

There is a need for novel methods for high-yield industrial production of recombinant proteins, e.g., alternative animal-free egg proteins.

### SUMMARY

The present invention addresses this need. The systems and methods provide high-titer expression of recombinant proteins in large scale production and are particularly useful for expressing heterologous proteins in a microbial host, such as food-based proteins or other protein types such as therapeutic proteins and enzymes.

In one aspect, provided herein are engineered host cells for expressing one or more heterologous genes, the engineered host cell comprising a plurality of expression cassettes integrated into the genome of the engineered host cell, the engineered host cell comprising: the plurality of expression cassettes each having two or more transcriptional elements, wherein at least one of the expression cassettes comprises a combination of a set of transcriptional elements that are non-native to the engineered host cell, and each of the plurality of expression cassettes lacks a sequence of the one or more heterologous genes, wherein the engineered host cell is capable of integrating a plurality of coding constructs into the expression cassette without requiring a nuclease enzyme, wherein each coding construct comprises the sequence of at least one of the heterologous genes and at least a sequence homologous to the expression cassette or a partial sequence thereof.

In some embodiments, the plurality of expression cassettes comprises at least two different expression cassettes integrated into the genome of the engineered host cell.

In accordance with any one of the embodiments, each of the plurality of expression cassettes does not comprise a nuclease targeting sequence.

In accordance with any one of the embodiments, the transcriptional elements non-native to the engineered host cell are selected from the group consisting of a promoter, a terminator sequence, a signal sequence, or combinations thereof.

In accordance with any one of the embodiments, one or more of the plurality of expression cassettes comprise an inducible promoter, a regulated promoter, a repressible promoter, and/or a constitutive promoter.

In accordance with any one of the embodiments, one or more of the plurality of coding constructs lacks a full-length promoter sequence, an operable promoter sequence, or a promoter sequence native to the engineered host cell.

In accordance with any one of the embodiments, at least one of the plurality of expression cassettes further comprises a unique barcode sequence.

In accordance with any one of the embodiments, the engineered host cell is a yeast cell.

In accordance with any one of the embodiments, two or more of the plurality of expression cassettes are integrated in different chromosomes in the engineered host cell's genome.

In some embodiments, two or more of the plurality of expression cassettes are integrated in the same chromosome in the engineered host cell's genome.

In some embodiments, two or more expression cassettes integrated in the same chromosome are oriented in opposite transcriptional directions.

In some embodiments, two or more expression cassettes integrated in the same chromosome are oriented in the same transcriptional direction.

In some embodiments, the engineered host cell is a bacterial cell.

In accordance with any one of the embodiments, at least two of the plurality of expression cassettes comprise different promoters, different secretion signal sequences, different terminator sequences, or combinations thereof.

In accordance with any one of the embodiments, the engineered host cell comprises one or more integrated helper expression cassettes comprising a promoter driving expression of a helper protein.

In one aspect, provided herein are methods of expressing one or more heterologous genes in an engineered host cell, the method comprising: introducing a plurality of coding constructs into the host cell, wherein the host cell comprises a genome having a plurality of integrated expression cassettes each lacking a sequence of the one or more heterologous genes, wherein each coding construct comprises a sequence of at least one of the one or more heterologous genes and a first 5′ recognition zone comprising at least a sequence homologous to the expression cassette or a partial sequence thereof; and incubating the engineered host cell and the plurality of coding constructs in conditions that allow homologous recombination of the one or more coding constructs comprising the sequence of one or more heterologous genes with the expression cassettes, thereby integrating the sequence of one or more heterologous genes into the engineered host cell genome; wherein at least one of the plurality of expression cassettes comprises two or more transcriptional elements that are non-native to the engineered host cell; wherein the engineered host cell is capable of integrating the sequence of the one or more heterologous genes into the expression cassette without requiring a nuclease enzyme.

In some embodiments, at least one of the plurality of coding constructs is vector-less.

In accordance with any one of the embodiments, each of the plurality of expression cassettes does not comprise a nuclease targeting sequence.

In accordance with any one of the embodiments, at least one of the plurality of coding constructs does not comprise an origin of replication for a plasmid or vector.

In accordance with any one of the embodiments, at least one of the plurality of coding constructs is a linear DNA fragment.

In accordance with any one of the embodiments, at least one of the plurality of coding constructs lacks regulatory elements operably linked to the coding sequence of the heterologous gene.

In accordance with any one of the embodiments, at least one of the plurality of coding constructs lacks a full-length promoter sequence, an operable promoter sequence, or a promoter sequence native to the engineered host cell.

In accordance with any one of the embodiments, the first 5′ recognition zone comprises at least 50 nucleotides located 5′ to the coding sequence of the heterologous gene.

In some embodiments, the sequence of the 5′ recognition zone is homologous to a portion of the promoter sequence or a signal peptide sequence in one or more of the plurality of expression cassettes.

In accordance with any one of the embodiments, at least one or each of the plurality of coding constructs comprises a first 3′ recognition zone comprising at least 50 nucleotides located 3′ to the coding sequence of the heterologous gene.

In some embodiments, the sequence of the 3′ recognition zone is homologous to portion of a terminator sequence in one or more of the plurality of expression cassettes.

In accordance with any one of the embodiments, at least two different coding constructs are transformed into the engineered host cell.

In some embodiments, the two different coding constructs comprise the coding sequence for the same heterologous gene but comprise a different 5′ or 3′ recognition zone flanking the coding sequence of the heterologous gene.

In accordance with any one of the embodiments, the introduction comprises transformation and the coding constructs are transformed into the engineered host cell simultaneously.

In accordance with any one of the embodiments, at least 3, 4, 5, 6, 7, 8, 9 or 10 different coding constructs are transformed into the engineered host cell simultaneously.

In accordance with any one of the embodiments, each coding construct comprises the coding sequence of the same heterologous gene.

In accordance with any one of the embodiments, the plurality of the coding constructs comprises the coding sequence of at least two different heterologous genes.

In accordance with any one of the embodiments, the transcriptional elements non-native to the engineered host cells are selected from the group consisting of a promoter, a terminator sequence, a signal sequence non-native to the host cell, or combinations thereof.

In some embodiments, at least one of the combinations of transcriptional elements comprises a promoter sequence non-native to the host cell.

In accordance with any one of the embodiments, one or more of the plurality of expression cassettes comprise an inducible promoter, a regulated promoter, a repressible promoter, and/or a constitutive promoter.

In accordance with any one of the embodiments, at least one promoter in the plurality of expression cassettes comprises a unique barcode sequence.

In accordance with any one of the embodiments, the engineered host cell is a yeast cell.

In accordance with any one of the embodiments, two or more of the plurality of expression cassettes are integrated in different chromosomes in the engineered host cell's genome.

In accordance with any one of the embodiments, two or more of the plurality of expression cassettes are integrated in the same chromosome in the engineered host cell's genome.

In some embodiments, two or more expression cassettes integrated in the same chromosome are oriented in opposite transcriptional directions.

In accordance with any one of the embodiments, two or more expression cassettes integrated in the same chromosome are oriented in the same transcriptional direction.

In accordance with any one of the embodiments, the engineered host cell is a bacterial cell.

In accordance with any one of the embodiments, at least two of the plurality of expression cassettes comprise different promoters, different secretion signal sequences, different terminator sequences, or combinations thereof.

In accordance with any one of the embodiments, the engineered host cell comprises one or more integrated helper expression cassettes comprising a promoter driving expression of a helper protein.

In some embodiments, the method further comprises removing one or more expression cassettes that do not comprise the coding construct from the engineered host cell after step (b).

In some embodiments, the removing of the one or more expression cassettes is performed by inducing a double stranded break in or near the expression cassette.

In some embodiments, the double stranded break is not induced by a Cas enzyme.

In accordance with any one of the embodiments, the method further comprises culturing the engineered host cell in fermentation media and measuring an amount of the protein expressed by the one or more heterologous genes.

In accordance with any one of the embodiments, the method further comprises incubating the engineered host cell in conditions that allow integration of a second plurality of expression cassettes into the engineered host genome wherein each of the second plurality of expression cassettes comprises two or more transcriptional elements selected from the group consisting of a promoter, signal sequence and terminator sequence; and wherein at least one of the expression cassettes in the second plurality comprises a set of transcriptional elements that are non-native to the engineered host cell.

In some embodiments, the method further comprises transforming a second plurality of coding constructs into the engineered host cell, each construct comprising a coding sequence for a heterologous gene; incubating the engineered host cell with the second plurality of coding constructs in conditions that allow homologous recombination of the second plurality of coding constructs with the engineered host cell, thereby integrating the second plurality of coding constructs into the engineered host cell.

In accordance with any one of the embodiments, the method further comprises sequencing the engineered host cell genome.

In one aspect, provided herein are methods of producing an engineered host cell comprising, transforming a plurality of expression cassettes into the engineered host cell, the plurality of expression cassettes each lacking a coding sequence of a heterologous gene to the engineered host cell; incubating the engineered host cell in conditions that allow integration of the plurality of different expression cassettes into the host genome; wherein at least one of the plurality of expression cassettes comprises two or more transcriptional elements non-native to the engineered host cell, wherein the transcriptional elements are selected from the group consisting of a promoter, signal sequence and terminator sequence; and wherein the engineered host cell is capable of integrating the coding sequence of the heterologous gene into the one or more expression cassettes without requiring a nuclease enzyme.

In some embodiments, each of the plurality of expression cassettes do not comprise a nuclease targeting sequence.

In some embodiments, the engineered host cell comprises at least one heterologous expression cassette capable of driving expression of a heterologous gene sequence in the engineered host cell.

In some embodiments, the engineered host cell comprises at least one heterologous expression cassette driving expression of a helper factor gene sequence.

In accordance with any one of the embodiments, the method further comprises mating the engineered host cell with a second host cell.

In some embodiments, the second host cell comprises a plurality of different expression cassettes driving expression of a heterologous gene sequence to the second host cell.

In some embodiments, the second host cell has an antibiotic resistance marker different from an antibiotic resistance marker in the engineered host cell.

In one aspect, provided herein are kits for preparing the engineered host cell in accordance with any one of the embodiments, the kit comprising a plurality of engineered host cells and a set of instructions for culturing the engineered host cells, at least, for expressing recombinant proteins and/or instructions for performing the method in accordance with any one of the embodiments.

In one aspect, provided herein are libraries of vectors for transformation of a host cell, the library of vectors comprises a plurality of expression cassettes wherein: (a) at least one of the plurality of expression cassettes comprises two or more transcriptional elements non-native to the engineered host cell, wherein the transcriptional elements are selected from the group consisting of a promoter and a terminator sequence, and, optionally, signal sequence; at least one of the expression cassettes comprises a combination of transcriptional elements that is non-native to the host cell; wherein one or more of the plurality of expression cassettes lacks a coding sequence of a protein heterologous to the host cell.

In one aspect, provided herein are libraries of more than one different engineered host cell lines, the library comprising a cell of each of the more than one different engineered host cell lines comprises a plurality of expression cassettes, wherein at least one of the plurality of expression cassettes comprises two or more transcriptional elements non-native to the engineered host cell, wherein the transcriptional elements are selected from the group consisting of a promoter and a terminator sequence, and, optionally, signal sequence; wherein one or more of the plurality of expression cassettes lacks a coding sequence of a protein heterologous to the host cell; wherein each one of the different engineered host cell lines in the library comprises a different combination of expression cassettes.

In some aspects, provided herein may be an engineered host cell for expressing a heterologous gene. In some embodiments, the engineered host cell may comprise a plurality of expression cassettes integrated into the genome of the engineered host cell. In some cases, each of the plurality of expression cassettes may comprise two or more transcriptional elements selected from the group consisting of a promoter and a terminator sequence, and, optionally, a signal sequence. In some cases, at least one of the expression cassettes may comprise a combination of transcriptional elements that may be non-native to the engineered host cell; and one or more of the plurality of expression cassettes lacks a coding sequence of the heterologous gene.

In some embodiments, the plurality of expression cassettes may comprise at least two different expression cassettes integrated into the genome of the engineered host cell.

In some embodiments, the plurality of expression cassettes may comprise at least 3, 4, 5, 6, 7, 8, 9, or 10 different expression cassettes integrated into the genome of the engineered host cell.

In some embodiments, at least one of the plurality of expression cassettes does not comprise a coding sequence of the heterologous gene operably linked to a transcriptional element.

In some embodiments, each of the plurality of expression cassettes lacks a coding sequence of the heterologous gene operably linked to a transcriptional element.

In some embodiments, at least one of the plurality of expression cassettes may comprise a non-native combination of transcriptional elements.

In some embodiments, each of the plurality of expression cassettes may comprise a non-native combination of transcriptional elements.

In some embodiments, at least one of the non-native combinations of transcriptional elements may comprise a promoter sequence non-native to the host cell.

In some embodiments, one or more of the plurality of expression cassettes comprise an inducible promoter, a regulated promoter, a repressible promoter, and/or a constitutive promoter.

In some embodiments, at least one of the non-native combination of transcriptional elements may comprise a signal sequence non-native to the host cell.

In some embodiments, at least one of the non-native combination of transcriptional may comprise a terminator sequence non-native to the host cell.

In some embodiments, at least one of the plurality of expression cassettes further may comprise a unique barcode sequence.

In some embodiments, each promoter in the plurality of expression cassettes may comprise a unique barcode sequence.

In some embodiments, the unique barcode sequence may be a 100-3000 base pair sequence.

In some embodiments, the engineered host cell may be a eukaryotic cell.

In some embodiments, the engineered host cell may be a yeast cell.

In some embodiments, two or more of the plurality of expression cassettes are integrated in different chromosomes in the engineered host cell's genome.

In some embodiments, two or more of the plurality of expression cassettes are integrated in the same chromosome in the engineered host cell's genome.

In some embodiments, two or more expression cassettes integrated in the same chromosome are oriented in opposite transcriptional directions.

In some embodiments, two or more expression cassettes integrated in the same chromosome are oriented in the same transcriptional direction.

In some embodiments, the engineered cell may be a prokaryotic cell.

In some embodiments, the engineered host cell may be a bacterial cell.

In some embodiments, at least two of the plurality of expression cassettes comprise different promoters.

In some embodiments, at least two of the plurality of expression cassettes comprise different signal sequences.

In some embodiments, at least two of the plurality of expression cassettes comprise different terminator sequences.

In some embodiments, the engineered host cell may comprise one or more integrated helper expression cassettes may comprise a promoter driving expression of a helper protein.

In some aspects, described herein are methods of expressing a heterologous gene in an engineered host cell. In some embodiments, the method may comprise: providing an engineered host cell may comprise a genome may comprise a plurality of integrated expression cassettes each lacking a coding sequence of a heterologous gene prior to the transformation; transforming the host cell with a plurality of coding constructs, wherein each construct may comprise a coding sequence of the heterologous gene; and incubating the engineered host cell obtained at the completion of step b in conditions that allow integration of one or more coding constructs via homologous recombination into the integrated expression cassettes. In some cases, each of the plurality of coding constructs may be vector-less and/or lacks regulatory elements when transformed. In some cases, each of the plurality of expression cassettes may comprise two or more transcriptional elements selected from the group consisting of a promoter, signal sequence and terminator sequence. In some cases, at least one of the expression cassettes comprise a combination of transcriptional elements that may be non-native to the engineered host cell.

In some embodiments, at least one of the plurality of coding constructs may be vector-less.

In some embodiments, at least one of the plurality of coding constructs may be double stranded DNA.

In some embodiments, at least one of the plurality of coding constructs may be single stranded DNA.

In some embodiments, at least one of the plurality of coding constructs may comprise a promoter sequence.

In some embodiments, at least one of the plurality of coding constructs does not comprise an origin of replication for a plasmid or vector.

In some embodiments, at least one of the plurality of coding constructs may be linear DNA fragment.

In some embodiments, at least one of the plurality of coding constructs lacks regulatory elements linked to the coding sequence of the heterologous gene.

In some embodiments, at least one of the plurality of coding constructs lacks a full-length promoter sequence.

In some embodiments, each of the plurality of coding constructs may comprise a first 5′ recognition zone may comprise at least 50 nucleotides located 5′ to the coding sequence of the heterologous gene.

In some embodiments, the sequence of the 5′ recognition zone may be homologous to portion of a promoter or a signal sequence in one or more of the plurality of expression cassettes.

In some embodiments, at least one or each of the plurality of coding constructs may comprise a first 3′ recognition zone may comprise at least 50 nucleotides located 3′ to the coding sequence of the heterologous gene.

In some embodiments, a sequence of the 3′ recognition zone may be homologous to portion of a terminator in one or more of the plurality of expression cassettes.

In some embodiments, at least two different coding constructs are transformed into the engineered host cell.

In some embodiments, the two different coding constructs comprise the coding sequence for the same heterologous gene but comprise a different 5′ or 3′ recognition zone flanking the coding sequence of the heterologous gene.

In some embodiments, the coding constructs are transformed into the engineered host cell simultaneously.

In some embodiments, at least 3, 4, 5, 6, 7, 8, 9 or 10 different coding constructs are transformed into the engineered host cell.

In some embodiments, at least 3, 4, 5, 6, 7, 8, 9 or 10 different coding constructs are transformed into the engineered host cell simultaneously.

In some embodiments, each coding construct may comprise the coding sequence of the same heterologous gene.

In some embodiments, the plurality of the coding constructs may comprise the coding sequence of at least two different heterologous genes.

In some embodiments, the engineered host cell provided in step a may comprise at least two different expression cassettes integrated into its genome.

In some embodiments, the engineered host cell provided in step a may comprise at least 3, 4, 5, 6, 7, 8, 9, or 10 different expression cassettes integrated into its genome.

In some embodiments, when the engineered host cell may be provided in step a, at least one of the plurality of expression cassettes does not comprise a coding sequence of the heterologous gene operably linked to a transcriptional element prior to the transformation.

In some embodiments, when the engineered host cell may be provided in step a, each of the plurality of expression cassette lacks a coding sequence of the heterologous gene operably linked to a transcriptional element prior to the transformation.

In some embodiments, at least one of the plurality of expression cassettes may comprise a non-native combination of transcriptional elements.

In some embodiments, each one of the plurality of expression cassettes comprise a non-native combination of transcriptional elements.

In some embodiments, at least one of the non-native combinations of transcriptional elements may comprise a promoter sequence non-native to the host cell.

In some embodiments, one or more of the plurality of expression cassettes comprise an inducible promoter, a regulated promoter, a repressible promoter, and/or a constitutive promoter.

In some embodiments, at least one of the non-native combinations of transcriptional elements may comprise a signal sequence non-native to the host cell.

In some embodiments, at least one of the non-native combinations of transcriptional may comprise a terminator sequence non-native to the host cell.

In some embodiments, at least one promoter in the plurality of expression cassettes may comprise a unique barcode sequence.

In some embodiments, each of the plurality of expression cassettes may comprise a unique barcode sequence.

In some embodiments, the unique barcode sequence may be a 100-3000 base pair sequence.

In some embodiments, the engineered host cell may be a eukaryotic cell.

In some embodiments, the engineered host cell may be a yeast cell.

In some embodiments, two or more of the plurality of expression cassettes are integrated in different chromosomes in the engineered host cell's genome.

In some embodiments, two or more of the plurality of expression cassettes are integrated in the same chromosome in the engineered host cell's genome.

In some embodiments, two or more expression cassettes integrated in the same chromosome are oriented in opposite transcriptional directions.

In some embodiments, two or more expression cassettes integrated in the same chromosome are oriented in the same transcriptional direction.

In some embodiments, the engineered cell may be a prokaryotic cell.

In some embodiments, the engineered host cell may be a bacterial cell.

In some embodiments, at least two of the plurality of expression cassettes comprise different promoters.

In some embodiments, at least two of the plurality of expression cassettes comprise different signal sequences.

In some embodiments, at least two of the plurality of expression cassettes comprise different terminator sequences.

In some embodiments, the engineered host cell may comprise one or more integrated helper expression cassettes may comprise a promoter driving expression of a helper protein.

In some embodiments, the method further may comprise removing one or more expression cassettes that do not comprise the coding construct from the engineered host cell after step (c).

In some embodiments, the removing of the one or more expression cassettes may be performed by inducing a double stranded break in or near the expression cassette.

In some embodiments, the double stranded break may be induced by a Cas9 enzyme.

In some embodiments, the method further may comprise culturing the engineered host cell in fermentation media and measuring an amount of the protein generated.

In some embodiments, the culturing the engineered host cell may comprise culturing the engineered host cell for at least 1 hour.

In some embodiments, the method further may comprise incubating the engineered host cell in conditions that allow integration of a second plurality of expression cassettes into the engineered host genome; wherein each of the second plurality of expression cassettes may comprise two or more transcriptional elements selected from the group consisting of a promoter, signal sequence and terminator sequence; and wherein at least one of the expression cassettes in the second plurality comprise a combination of transcriptional elements that may be non-native to the engineered host cell.

In some embodiments, the method further may comprise transforming a second plurality of coding constructs into an engineered host cell, each construct may comprise a coding sequence for a heterologous gene; incubating the engineered host cell in conditions that allow integration of one or more coding constructs of the second plurality via homologous recombination into one or more of the second plurality of expression constructs that are integrated into genome of the engineered host cell.

In some embodiments, the method further may comprise sequencing the engineered host cell genome.

In some embodiments, method further may comprise incubating the engineered host cell in conditions that allow integration of a second plurality of expression cassettes into the engineered host genome; wherein each of the second plurality of expression cassettes may comprise two or more transcriptional elements selected from the group consisting of a promoter, signal sequence, and terminator sequence; and wherein at least one of the expression cassettes in the second plurality comprise a combination of transcriptional elements that may be non-native to the engineered host cell.

In some embodiments, the method further may comprise transforming a second plurality of coding constructs into a host cell, each construct may comprise a coding sequence for a heterologous gene; incubating the engineered host cell in conditions that allow integration of one or more coding constructs of the second plurality via homologous recombination into one or more of the second plurality of expression constructs that are integrated into genome of the engineered host cell.

In some aspects, provided herein are methods of producing an engineered host cell. In some embodiments, the method may comprise, transforming a plurality of expression cassettes lacking a protein coding sequence into the engineered host cell and incubating the engineered host cell in conditions that allow integration of the plurality of different expression cassettes into the host genome. In some embodiments, each of the plurality of expression cassettes may comprise two or more transcriptional elements selected from the group consisting of a promoter, signal sequence and terminator sequence. In some embodiments, at least one of the expression cassettes comprise a combination of transcriptional elements that may be non-native to the engineered host cell.

In some embodiments, the engineered host cell may be a naïve host cell.

In some embodiments, the engineered host cell may comprise one or more genetic modification prior to the transforming of the plurality of expression cassettes lacking a protein coding sequence.

In some embodiments, the engineered host cell has one or more genes knocked out.

In some embodiments, the engineered host cell may comprise at least one heterologous expression cassette capable of driving expression of a protein coding sequence.

In some embodiments, the engineered host cell may comprise at least one heterologous expression cassette driving expression of a helper factor gene sequence.

In some embodiments, the method further may comprise mating the engineered host cell with a second host cell.

In some embodiments, the second host cell may comprise a plurality of different expression cassettes driving expression of a heterologous protein coding sequence, optionally, wherein the second host cell may be created by a method of any one of claims 27 to 81.

In some embodiments, the second host cell has an antibiotic resistance marker.

In some embodiments, the antibiotic resistance marker in the second host cell may be different from an antibiotic resistance marker in the host cell of any one of claims 82 to 87.

In some aspects, provided herein is a kit comprising a plurality of engineered host cells described herein and a set of instructions for culturing the engineered host cells, at least, for expressing recombinant proteins and/or instructions for performing the methods described herein.

In some aspects, provided herein is a library of vectors for transformation of a host cell, wherein the library of vectors may comprise a plurality of expression cassettes. In some embodiments, each of the plurality of expression cassettes may comprise two or more transcriptional elements selected from the group consisting of a promoter and a terminator sequence, and, optionally, signal sequence. In some embodiments, at least one of the expression cassettes may comprise a combination of transcriptional elements that may be non-native to the host cell. In some embodiments, one or more of the plurality of expression cassettes lacks a coding sequence of a protein heterologous to the host cell.

In some aspects, provided herein is a library of more than one different engineered host cell lines, wherein a cell of each of the more than one different engineered host cell lines may comprise a plurality of expression cassettes. In some embodiments, each of the plurality of expression cassettes may comprise two or more transcriptional elements selected from the group consisting of a promoter and a terminator sequence, and, optionally, signal sequence. In some embodiments, at least one of the expression cassettes may comprise a combination of transcriptional elements that may be non-native to the host cell. In some embodiments, wherein one or more of the plurality of expression cassettes lacks a coding sequence of a protein heterologous to the host cell. In some embodiments, wherein each one of the different engineered host cell lines in the library may comprise a different combination of expression cassettes.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates examples of expression cassettes that may be used in the methods described herein.

FIG. 1B provides a schematic representation of a host cell with an integrated expression cassette which ultimately comprises the coding sequence of a heterologous gene.

FIG. 1C provides a schematic representation of integration of a coding construct into the integrated expression cassette in a host cell.

FIG. 2A-D shows illustrative schematics of integrated expression cassettes in a host cell.

FIG. 3A shows illustrative approaches to generating recombinant protein producing strains.

FIG. 3B illustrates a strategy to generate a hybrid strain combining elements from two strains with multiple integrated expression cassettes.

FIG. 3C illustrates a strategy to generate a host cell with a balanced or desired expression profile.

FIG. 3D illustrates a strategy for generating high protein expression strains using Crispr/Cas9 to target sites with expression cassettes that do not contain a coding sequence for a gene of interest. Figure discloses SEQ ID NOS 43-45, respectively, in order of appearance.

FIGS. 4A-D illustrate multicopy expression cassettes in vector backbones.

FIG. 5 shows mCherry fluorescence in cell free supernatants.

FIG. 6 shows SDS-PAGE analysis of mCherry secretion.

FIG. 7 shows results of a genetic marker stability test.

FIG. 8 shows diagnostic PCR results for mCherry expression.

FIG. 9A illustrates the array of integrated cassettes in an illustrative host cell expressing mCherry.

FIG. 9B illustrates the PCR product sequence matched up to an expression cassette in the illustrative host cell. Figure discloses SEQ ID NOS 46-47, respectively, in order of appearance.

FIG. 9C illustrates a diagnostic PCR schematic.

FIG. 10A illustrates the array of integrated cassettes in another illustrative host cell expressing mCherry.

FIGS. 10B-C show a close-up of the integrated expression constructs with successful integration events of mCherry.

FIGS. 11A-B illustrate base-calling errors observed in genomic DNA sequences. Figures discloses SEQ ID NOS 48-53, 48, 54-56, 50, 57-62, 50, 63-64, 59, and 65, respectively, in order of appearance.

FIGS. 12A-B illustrate titer distribution of goi producing host cells.

FIG. 12C shows the results of SDS-PAGE analysis of goi.

FIG. 13 shows results of SDS-PAGE analysis of GOI expression.

FIG. 14 shows titer distribution of GOI expressing cells.

FIGS. 15A-B show results of SDS-PAGE analysis of GOI expression.

FIGS. 16A-B illustrate titers of GOI producing stains.

FIGS. 17A-B illustrate expression cassette integration via PCR and SDS-PAGE analysis of goi expressing transformants.

FIG. 18 illustrates distribution of goi-expressing transformants.

FIG. 19 illustrates SDS-PAGE results of goi expression in transformants.

FIG. 20 illustrates 0 hr titers of goi producing strains.

FIG. 21A-D illustrate strain schematics of integrated expression cassettes in host cells.

FIG. 22 illustrates titers of host cells producing rEWP4 during fermentation.

### DETAILED DESCRIPTION

Provided herein are biological systems and methods for production of proteins, such as food-related proteins using regulatory control in engineered host cells such as yeast and bacteria. The biological systems and methods described herein employ integration of heterologous expression constructs with or without gene sequences. The systems and methods described herein increase the speed and efficiency of expressing heterologous genes of interest in host cells with high titers. The systems and methods described herein provide a modular approach towards expression of a variety of proteins in host cells where the In some embodiments, the integrated heterologous sequence encodes for a food-based or food-related protein, such as one that is used as a food ingredient or for processing to make a food product.

The systems and methods provided herein are designed for engineering of a host cell by introducing into the host cell, heterologous sequences or a heterologous combination of regulatory elements comprised within one or more expression cassettes. In an initial round of engineering the host cell, the expression cassettes integrated can lack a coding sequence for any genes of interest. One of the various technical advantages of using this approach to host cell engineering is to generate a host cell which is capable of integrating any gene into the cell without the need for cloning multiple vectors in vitro.

The following disclosure describes systems and methods of driving high expression of a heterologous protein in a host cell by combining (i) stable integration of a plurality of expression cassettes driven by a diverse set of promoters, each integration site carrying a plurality of copies of one or more expression cassettes; (ii) co-transformation of the multiple expression cassettes into the genome of a host cell, using non-homologous recombination methods; and (iii) transformation of coding constructs comprising the coding sequences of genes of interest post-integration of the expression cassettes in the host cell genome. The integration of expression cassettes with diverse promoters can overcome potential issues with multiple copy integration such as possible depletion of cognate transcription factors that are required for the expression of the cassettes and the potential for deletion of copies through recombination events or other host mechanisms. The integration of expression cassettes without the coding sequences of genes of interest allows for the use of a high titer producing genomic background host cell for more than one gene and reduce the need for cloning multiple vectors for each gene of interest.

In some embodiments, after integration, the engineered cells described herein do not contain sequences encoding selectable markers such as auxotrophic markers or antibiotic resistance genes, thereby reducing the amount of extraneous heterologous DNA that is integrated into the host genome. Additionally, because many auxotrophic markers are highly homologous to endogenous genes in the host cell, the use of such markers may favor homologous recombination of the transformed DNA.

The systems and methods herein are particularly useful for producing nutritive proteins, e.g., plant or animal proteins for food ingredients and products, with applications in food and health, as well as animal-derived proteins for food production, because of the improved capability for high-titer expression in large-scale settings as well as a ‘cleaner’ production system without the utilization of antibiotic or other selection markers.

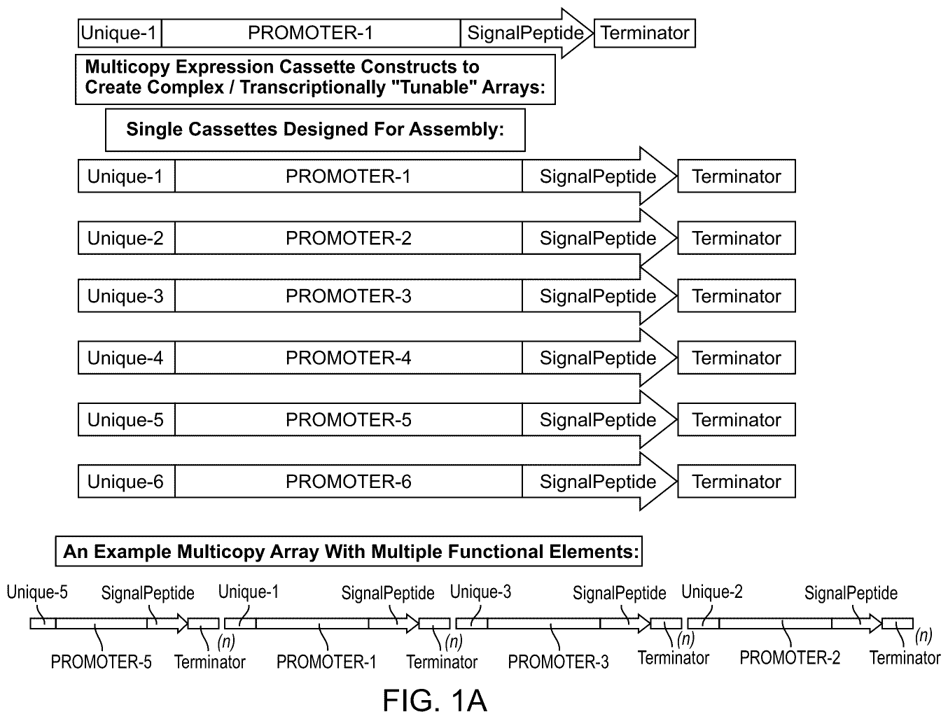

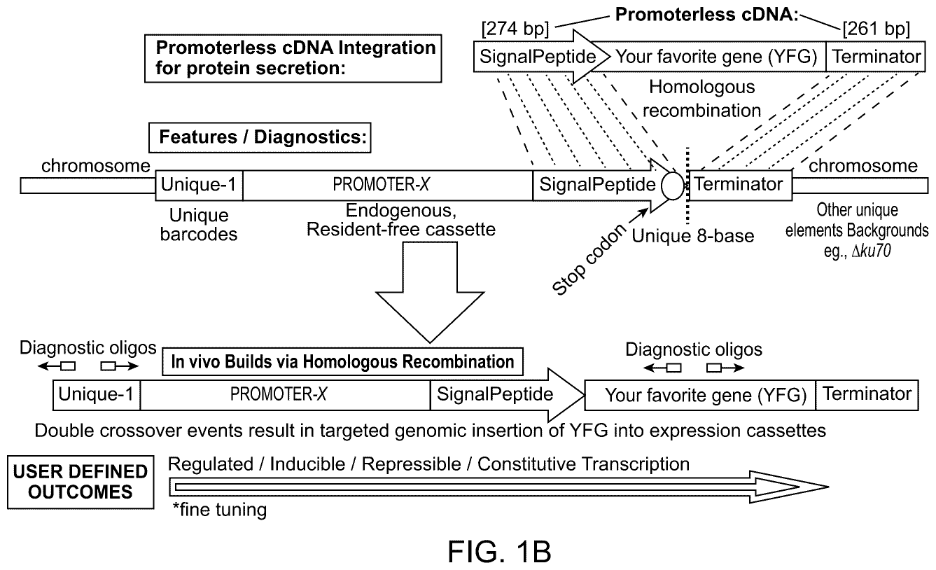

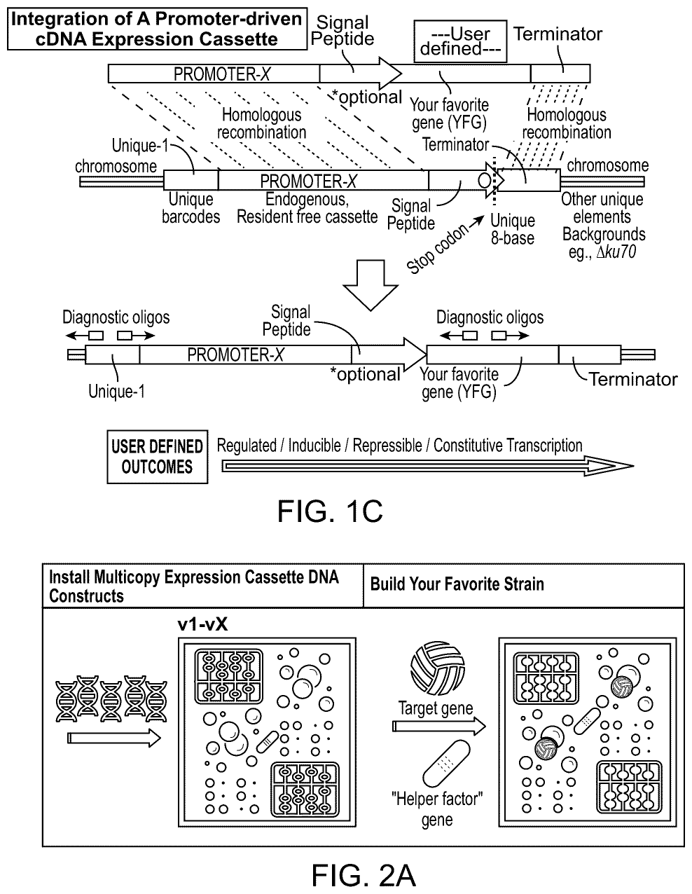

Provided herein are illustrative methods for generating host cells described herein. FIGS. 1A-C provide an illustrative schematic of the method. As shown in FIG. 1A, multiple expression cassettes may be generated wherein each cassette comprises a different promoter. In this example, 6 different expression cassettes were designed, each with a unique barcode sequence, a different promoter, a signal peptide and a terminator sequence. These cassettes can be introduced into the host cell using various methods including plasmid transformation. In some cases, one or more cassettes may comprise a sequence that directs integration of the cassette into a defined locus. Alternatively, the one or more cassettes may be allowed to randomly integrate into the host cell. FIG. 1A also shows an illustrative array of expression cassettes integrated into the host genome.

FIG. 1B shows an illustrative schematic of integration of a coding construct comprising the coding sequence of your favorite gene (YFG) into the already integrated expression cassettes in the host cell. The coding construct in this example comprises 2 different recognition zones in the form of a 274 bp signal peptide sequence at the 5′ end of the YFG coding sequence and a 261 bp terminator sequence at the 3′ end of the YFG coding sequence. The coding construct here lacks a promoter sequence. Such a coding construct can be transformed or transfected in the host cell as a linear DNA sequence as well as part of the plasmid. Homologous recombination allows for the integration of the YFG containing coding construct into all of the expression cassettes as they may have the same signal and/or terminator sequences. In other examples, the signal sequences and/or the terminator sequences may be different and therefore the coding construct can be directed towards specific expression cassettes. This can be especially helpful in generating strains with user defined outcomes. For instance, a user can select a constitutive promoter for YFG expression or a regulated promoter, an inducible promoter or a repressible promoter.

This method may be used to produce multiple proteins. For instance, YFG1 and YFG2 can be expressed in the same host cell. The recognition zones for YFG1 can be directed by constitutive promoter containing cassettes whereas recognition zones for YFG2 can be directed by inducible promoter containing cassettes. In this example, YFG2 can be expressed for a portion of the culturing time while a mixture of both YFG1 and YFG2 occurs throughout the culturing time. Other such permutations and combinations of elements are also envisioned. The methods, engineered host cells, and kits described herein are not restricted to any type of protein or promoter or host cell, thereby, allowing extensive options in for protein expression.

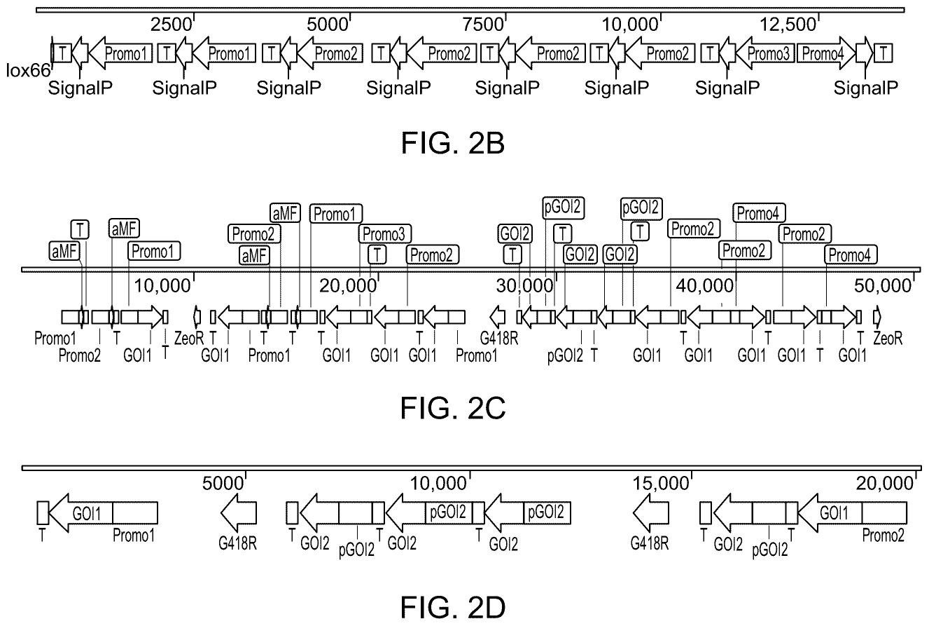

FIG. 2A provides an example of the methods described herein. Multiple copies of expression cassettes (shown as different colored helices), each different from each other (for instance, with different promoters or a combination of elements) can be installed in a host cell. The host cell may be a host cell with no genetic modifications, a host cell which has expression cassettes driving expression of a gene or a host cell with any other genetic modifications. The target gene or the gene of interest may be transformed into the host cell with the integrated expression cassettes as shown in FIG. 2A. In addition, in some cases, a helper factor gene sequence may also be transformed into a host cell with expression cassettes integrated. FIG. 2B provides an illustrative schematic of an array formed by integration of expression cassettes in a host cell. FIG. 2C shows illustrative results from a single round of transformation of a gene of interest (GOI). As shown, a single round of transformation can lead to multiple successful integrations of GOI in the cassettes. Of note is the integration of expression cassettes in various different orientations and directions. Additionally, shown in FIG. 2D are illustrative random integrations of the coding constructs in the host cell without the presence of an expression cassette. It was noted that a single round of transformation of the gene of interest into strains with or without the expression cassettes already integrated showed higher copies integrated in the strains with the expression cassettes (such as the one showed in FIG. 2C) and also led to titers at least twice the titers of strains where expression cassettes were not present (such as the one shown in FIG. 2D).

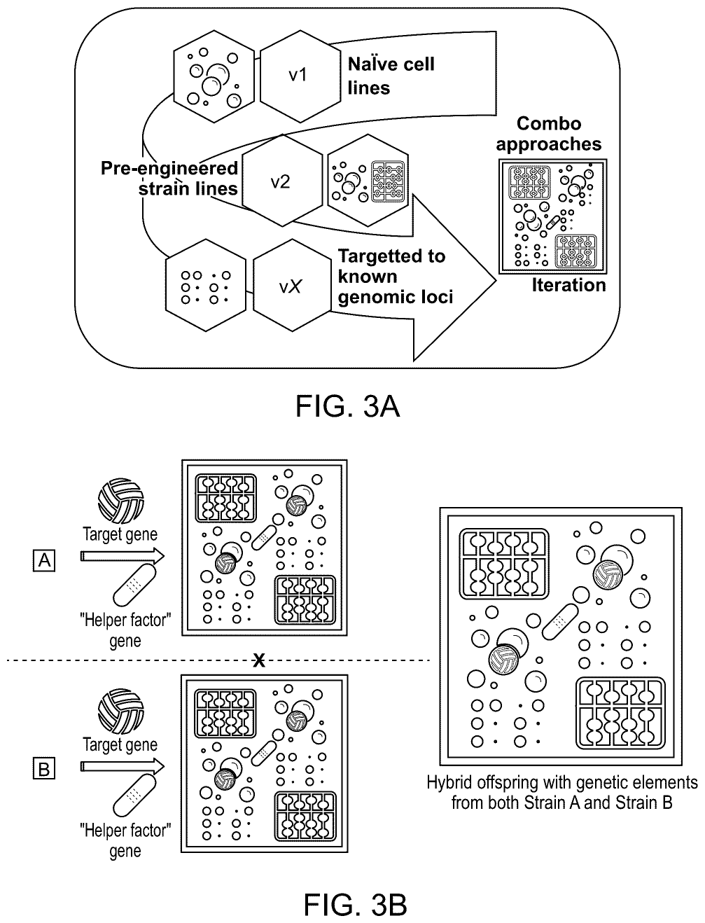

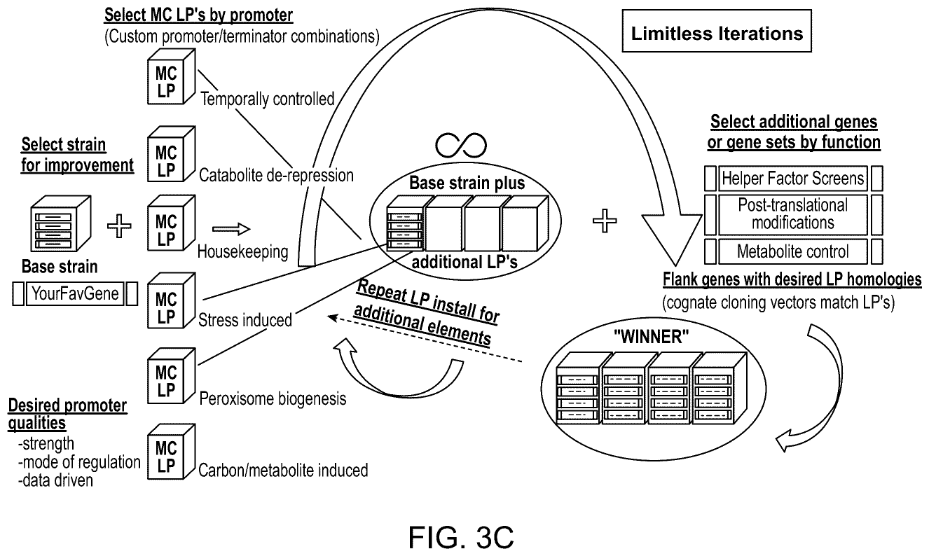

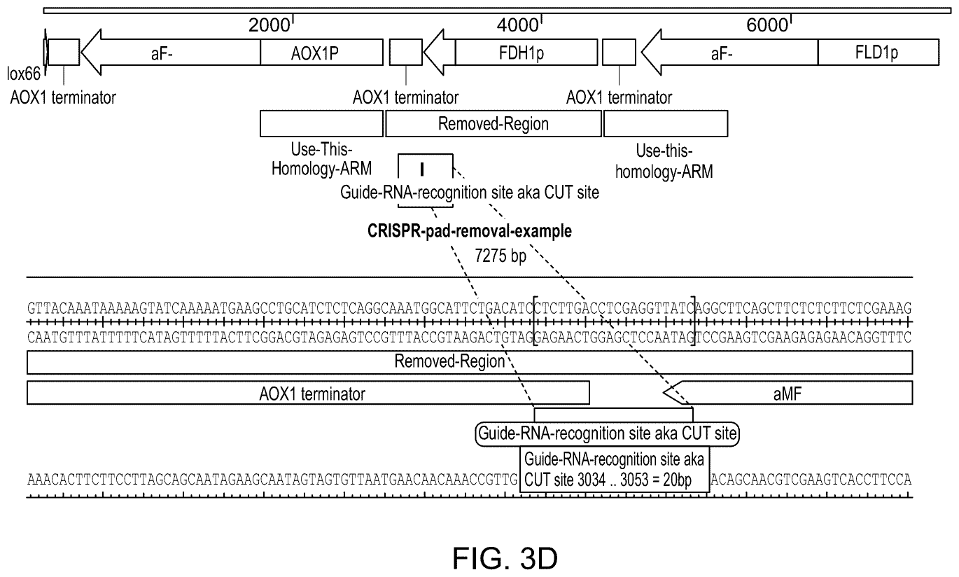

The methods described herein also allow for an iterative approach to producing strains with high titers and stable integrations. In one example, a host cell, such as the one shown in FIG. 2B comprising various integrations of different expression cassettes may be transformed with coding constructs directing integration using common signal sequence and terminator sequences as recognition zones. This transformation may result in a high titer producing strain with a balanced use of expression cassettes and promoters (for instance, if each type of expression cassette with a different promoter sequence can have the coding sequence integrated). For instance, as described in FIG. 3A, a first set of expression cassettes may be integrated into a naïve cell line. Such a transformation may be followed by a second round of integration with different expression cassettes or multiple rounds of transformation of the gene of interest. In some cases, expression cassettes may be integrated into pre-engineered strain lines. Such strains may be pre-engineered to comprise expression cassettes driving expression of heterologous genes, strains with one or more genes knocked out, other genetic modifications such as promoter modifications, etc.

If a higher titer or a more balanced expression profile is desired, an illustrative method may comprise mating two different host cells with different profiles as shown in FIG. 3B. For instance, a first round of transformation in host cell A can favor the integration of a gene in expression cassettes A, B and C whereas in host cell B can favor the integration of a gene in expression cassettes B, D and E, the two host cells A and B can be mated to produce an offspring containing coding sequence integrations in all cassettes. This may be further facilitated by the presence of different resistance markers in each expression cassette for easier selection of viable offspring.

Alternatively, one or more subsequent rounds of transformations may be used with specific coding constructs depending on the results of the first round. For example, FIG. 2C shows the illustrative results from a first round of transformations. As is shown in FIG. 2C, many of the expression cassettes with Promoters 1 and 2 did not integrate the GOI in the first round of transformation. To maximize expression efficiency, a user can design coding constructs that have the promoter 1 or 2 sequence as the recognition zone and direct homologous recombination into those cassettes.

In another example, a user may be able to take the host cell of FIG. 2C and integrate additional expression cassettes comprising promoters 5 and 6 to diversify the promoter population. These cassettes 5 and 6 can be allowed to randomly integrate in the host cell or their integration can be directed to avoid the disruption of the existing cassettes. For example, if cassettes 1, 2, 3 and 4 are integrated into chromosome 2, the integration of cassettes 5 and 6 can be directed to chromosome 4. The coding constructs in this example can be designed to have homology to promoters 5 and 6 to avoid their integration into any other cassette. In another variation of this method, the host cell of FIG. 2C can be transformed with plasmids comprising a complete expression cassette with promoters 5 and 6 driving expression of the GOI. These plasmids can be allowed to integrate randomly or directed to certain loci in the genome as explained above.

For instance, one or more expression cassettes comprising promoter 1 may be directed to a locus with higher availability for integration. This may be achieved by adding homology to a more available locus. For example, an expression cassette with a PMP47 promoter may have a fragment of the AOX1 gene/promoter so the PMP47 expression cassette may be able to integrate into the AOX1 locus.

In another example, to achieve a higher titer strain or a more balanced expression profile, a user may be able to design a strain as illustrated in FIG. 3C. For instance, a strain may be transformed with one or more expression cassettes (designated as LPs in FIG. 3C) wherein the expression cassettes express a gene of interest. The same cell can also be transformed with one or more multicopy (MC) expression cassettes which have promoters directed to various life stages of a host cell. For instance, the promoters in an expression cassette may be one or more temporally controlled promoters (for instance, the TLR1, GND2 or RPL40B promoters, one or more catabolite de-repression promoters (for instance the FMD, TKL3, RGIP, TMA10, MH2 promoters, promoters P1-P7 from Table 1), one or more housekeeping promoters (for instance, a GPI-anchored cell wall protein promoter like GCW14), one or more stress-induced promoters (for instance the unfolded protein response (UPR) transcriptional regulator HAC1 promoter, MH2 promoter), one or more peroxisome biogenesis promoters (for instance as Pex11, PMP20, PMP47 promoters), one or more carbon/metabolite induced/repressed promoters (for instance, methanol inducible or glucose repression/de-repression promoters such as AOX1, DAS1, DAS2, FGH1, FDH1, FLD1, TKL3, RGIP, TMA10, MH2 promoters, promoters P1-P7 from Table 1) or other such promoters. Such host cells can be tested for expression profiles and further modified as required. In some cases, if a host cell activates a stress response due to heterologous protein expression a new set of expression cassettes may be introduced into the host cell which have stress-inducible promoters and a new expression construct relative to the expression cassette may be introduced to drive the expression of the heterologous protein. This technique may maximize protein expression by a host cell.

In another example, a user may be able to use tools such as CRISPR/Cas9 to direct homologous recombination of a gene of interest into expression cassettes that did not end up with the integration of the gene of interest after a first round of transformation. A “recognition sequence” such as one shown as the 20 bp sequence shown in FIG. 3D for CRISPR/Cas9 which spans the alpha-MF (αMF) and AOX1 transcriptional terminator junction, present only in empty expression cassettes may be used for modifications. Such a Cas9 recognition sequence (or similar empty expression cassette “signatures” such as ones in promoter-terminator junctions) may allow the Cas9 enzyme to cut next to the recognition sequence and create a double-stranded break. Once the break is created at this site, the repair fragment (black) may enable the cell to repair the break via homologous recombination. The repair may lead to the removal of an empty expression cassette. Alternatively, the repair may lead to the integration of the gene of interest into the expression cassette.

In another example, an engineered host cell as described herein, comprising multiple expression cassettes may be used to produce a complex protein or multiple proteins or peptides in a biosynthetic pathway. For instance, individual components such as subunits of a protein or units of a biosynthetic pathway may be expressed in the same host cell. Different units (of one protein or different proteins or peptides of a pathway) may be produced at different expression levels as well. For instance, the coding sequence of one unit may be targeted to an expression cassette with a high producing constitutive promoter. The coding sequence of a second unit, less required than the first one may be targeted to an expression cassette with a low expressing promoter or an inducible promoter. By virtue of the differences in respective promoter strengths, users are able to balance the expression of individual components of these complexes and potentially balance critical components stoichiometrically.

### Expression Cassettes

One or more expression cassettes may be integrated into a host cell. One or more expression cassettes described herein may comprise one or more transcriptional elements. Transcriptional elements may include a promoter, a signal sequence, a terminator sequence, etc. In some cases, a host cell comprises at least 1, 2, 3, 4, 5, 6, 7, 8, 9, 10, 12, 14, 16, 18, or 20 integrated expression cassettes wherein each integrated cassette comprises at least a promoter sequence and a terminator sequence. In some cases, a host cell comprises at least 1, 2, 3, 4, 5, 6, 7, 8, 9, 10, 12, 14, 16, 18, or 20 integrated expression cassettes wherein each integrated cassette comprises at least a promoter sequence, a signal sequence and a terminator sequence.

A high number of expression cassettes may be integrated into a host cell. One or more expression cassettes described herein may comprise one or more transcriptional elements. Transcriptional elements may include a promoter, a signal sequence, a terminator sequence, etc. In some cases, a host cell comprises at least 10, 20, 30, 40, 50, 60, 70, 80, 90, 100, 120, 140, 160, 180, or 200 integrated expression cassettes wherein each integrated cassette comprises at least a promoter sequence and a terminator sequence. In some cases, a host cell comprises at least 10, 20, 30, 40, 50, 60, 70, 80, 90, 100, 120, 140, 160, 180, or 200 integrated expression cassettes wherein each integrated cassette comprises at least a promoter sequence, a signal sequence and a terminator sequence.

One or more unique expression cassettes may be integrated into a host cell. Each unique expression cassette described herein may comprise a unique combination of one or more transcriptional elements. Transcriptional elements may include a promoter, a signal sequence, a terminator sequence, etc. In some cases, a host cell comprises at least 1, 2, 3, 4, 5, 6, 7, 8, 9, 10, 12, 14, 16, 18, or 20 integrated unique expression cassettes wherein each integrated unique cassette comprises at least a promoter sequence and a terminator sequence. In some cases, a host cell comprises at least 1, 2, 3, 4, 5, 6, 7, 8, 9, 10, 12, 14, 16, 18, or 20 integrated unique expression cassettes wherein each integrated unique cassette comprises at least a promoter sequence, a signal sequence and a terminator sequence.

One or more expression cassettes integrated into a host cell may comprise transcriptional elements that are heterologous or non-native to the host cell. For instance, in some cases, a promoter sequence in the one or more expression cassettes may be heterologous to the host cell. In some cases, a signal sequence in the one or more expression cassettes may be heterologous to the host cell. In some cases, a terminator sequence in the one or more expression cassettes may be heterologous to the host cell. Alternatively, a combination of transcriptional elements may be heterologous or non-native to the host cell. For instance, an expression cassette may comprise a promoter sequence with a terminator sequence wherein the combination of the promoter sequence and terminator sequence is non-native to the host cell. In one example for instance, a yeast FDH1 promoter may be combined with a terminator sequence from the AOX1 gene in the expression cassette. In some cases, 1, 2, 3, 4, 5, 6, 7, 8, 9 or 10 of the expression cassettes integrated in the host cell may comprise a non-native or heterologous combination of transcriptional elements. In some cases, all of the expression cassettes introduced and integrated in the host cell may comprise a non-native or heterologous combination of transcriptional elements.

In some cases, the one or more expression cassettes integrated into a host cell may comprise transcriptional elements that are native to the host cell. Additionally, a combination of transcriptional elements may be heterologous to the host cell and may be native to the host cell.

The one or more expression cassettes integrated into the genome of the host cell lack a coding sequence for a gene of interest. In such cases, the gene of interest may be a gene heterologous to the host cell. In some cases, all of the heterologous expression cassettes integrated into the host cell lack a coding sequence of a gene of interest. In some cases, 1, 2, 3, 4, 5, 6, 7, 8, 9 or 10 of the expression cassettes integrated in the host cell lack a coding sequence of a heterologous gene. Alternatively, the gene of interest may be native to the host cell.

The expression cassettes described herein may comprise unique barcode sequences. A unique barcode sequence may be placed before the promoter sequence in the expression construct. A unique barcode sequence may be placed after the terminator sequence in the expression construct. In some cases, an expression construct comprises unique barcode sequences before the promoter sequence and after the terminator sequence. The unique barcode sequences may be used as a diagnostic tool to detect successful integration events in the host cell.

In some cases, a unique barcode sequence may be from 6 base pairs (bp) to 20 bp. In some cases, a unique barcode sequence may be at least 6 bps long. In some cases, a unique barcode sequence may be at most 20 bp long. In some cases, a unique barcode sequence may be from 6 bp to 8 bp, 6 bp to 10 bp, 6 bp to 12 bp, 6 bp to 14 bp, 6 bp to 16 bp, 6 bp to 18 bp, 6 bp to 20 bp, 8 bp to 10 bp, 8 bp to 12 bp, 8 bp to 14 bp, 8 bp to 16 bp, 8 bp to 18 bp, 8 bp to 20 bp, 10 bp to 12 bp, 10 bp to 14 bp, 10 bp to 16 bp, 10 bp to 18 bp, 10 bp to 20 bp, 12 bp to 14 bp, 12 bp to 16 bp, 12 bp to 18 bp, 12 bp to 20 bp, 14 bp to 16 bp, 14 bp to 18 bp, 14 bp to 20 bp, 16 bp to 18 bp, 16 bp to 20 bp, or 18 bp to 20 bp long. In some cases, a unique barcode sequence may be 6 bp, 8 bp, 10 bp, 12 bp, 14 bp, 16 bp, 18 bp, or 20 bp long. In some cases, a unique barcode sequence may be at least 6 bp, 8 bp, 10 bp, 12 bp, 14 bp, 16 bp, 18 bp, or 20 bp long. In some cases, a unique barcode sequence may be at most 6 bp, 8 bp, 10 bp, 12 bp, 14 bp, 16 bp, 18 bp, or 20 bp long.

In some cases, a unique barcode sequence may be from 15 bp to 500 bp. In some cases, a unique barcode sequence may be at least 15 bp. In some cases, a unique barcode sequence may be at most 500 bp. In some cases, a unique barcode sequence may be 15 bp to 30 bp, 15 bp to 50 bp, 15 bp to 100 bp, 15 bp to 150 bp, 15 bp to 200 bp, 15 bp to 300 bp, 15 bp to 400 bp, 15 bp to 500 bp, 30 bp to 50 bp, 30 bp to 100 bp, 30 bp to 150 bp, 30 bp to 200 bp, 30 bp to 300 bp, 30 bp to 400 bp, 30 bp to 500 bp, 50 bp to 100 bp, 50 bp to 150 bp, 50 bp to 200 bp, 50 bp to 300 bp, 50 bp to 400 bp, 50 bp to 500 bp, 100 bp to 150 bp, 100 bp to 200 bp, 100 bp to 300 bp, 100 bp to 400 bp, 100 bp to 500 bp, 150 bp to 200 bp, 150 bp to 300 bp, 150 bp to 400 bp, 150 bp to 500 bp, 200 bp to 300 bp, 200 bp to 400 bp, 200 bp to 500 bp, 300 bp to 400 bp, 300 bp to 500 bp, or 400 bp to 500 bp. In some cases, a unique barcode sequence may be about 15 bp, 30 bp, 50 bp, 100 bp, 150 bp, 200 bp, 300 bp, 400 bp, or 500 bp. In some cases, a unique barcode sequence may be at least 15 bp, 30 bp, 50 bp, 100 bp, 150 bp, 200 bp, 300 bp or 400 bp. In some cases, a unique barcode sequence may be at most 30 bp, 50 bp, 100 bp, 150 bp, 200 bp, 300 bp, 400 bp, or 500 bp.

In some cases, a unique barcode sequence may be from 20 bp to 2,000 bp. In some cases, a unique barcode sequence may be from at least 20 bp. In some cases, a unique barcode sequence may be from at most 2,000 bp. In some cases, a unique barcode sequence may be from 20 bp to 50 bp, 20 bp to 100 bp, 20 bp to 300 bp, 20 bp to 500 bp, 20 bp to 1,000 bp, 20 bp to 1,500 bp, 20 bp to 2,000 bp, 50 bp to 100 bp, 50 bp to 300 bp, 50 bp to 500 bp, 50 bp to 1,000 bp, 50 bp to 1,500 bp, 50 bp to 2,000 bp, 100 bp to 300 bp, 100 bp to 500 bp, 100 bp to 1,000 bp, 100 bp to 1,500 bp, 1M bp to 2,000 bp, 300 bp to 500 bp, 300 bp to 1,000 bp, 300 bp to 1,500 bp, 300 bp to 2,000 bp, 500 bp to 1,000 bp, 500 bp to 1,500 bp, 500 bp to 2.000 bp, 1,000 bp to 1,500 bp, 1,000 bp to 2,000 bp, or 1,500 bp to 2,000 bp. In some cases, a unique barcode sequence may be from 20 bp, 50 bp, 100 bp, 300 bp, 500 bp, 1,000 bp, 1,500 bp, or 2,000 bp.

### Expression Cassette Integration Copy Number

In some embodiments, a plasmid for integration into the host cell can comprise one or multiple expression cassettes or one or more copies of one expression cassette. In some embodiments, a plasmid for integration into the host cell can comprise one or multiple copies of a first expression cassette and one or multiple copies of a second expression cassette.

In some cases, an engineered host cell can integrate one or more plasmids, with each plasmid comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 copies, at least 25 copies, at least 30 copies, at least 40 copies, at least 50 copies, at least 60 copies, at least 70 copies or at least 100 copies of a first expression cassette. In some cases, an engineered host cell can integrate one or more plasmids, with each plasmid comprising at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20 copies at least 25 copies, at least 30 copies, at least 40 copies, at least 50 copies, at least 60 copies, at least 70 copies or at least 100 copies of a first expression cassette.

In some cases, an engineered host cell can integrate one or more plasmids, each plasmid comprising at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 copies, at least 25 copies, at least 30 copies, at least 40 copies, at least 50 copies, at least 60 copies, at least 70 copies or at least 100 copies of a second expression cassette. In some cases, an engineered host cell can integrate one or more plasmids, each plasmid comprising at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20 copies, at least 25 copies, at least 30 copies, at least 40 copies, at least 50 copies, at least 60 copies, at least 70 copies or at least 100 copies of a second expression cassette.

In some cases, an engineered host cell can integrate one or more copies of a first expression cassette, one or more copies of a second expression cassette, and optionally one or more copies of a third expression cassette. In some cases, the host cell can integrate one or more copies of 1, 2, 3, 4, 5, 6, 7, 8, 9, 10, 15, 20 or 25 expression cassettes.

In some embodiments, the one or more expression cassettes lack a coding sequence for a gene of interest or a heterologous gene. In some cases, one or more copies of the one or more expression constructs lack a coding sequence of a gene of interest or a heterologous gene.

### Promoter Diversity

It is possible that bottle necks of transcription arise from depletion of the pool of those cognate transcription factors that are available to mediate the activity of the promoter in the one or more expression cassettes. In some embodiments, a variety of expression cassettes are introduced, with each expression cassette carrying a different promoter thus diversifying the demand on available transcription factors to drive expression. Additionally, the use of multiple promoters reduces the homology between cassettes, which may increase stability of integration and copy number, particularly when multiple copies are integrated together at a site within the genome.

In some embodiments, the one or more expression cassettes contain different promoter sequences. The promoters can be derived from different sources (e.g., different regulatory regions). The promoters can be derived from the same or substantially similar sources but different in overall length of sequence and/or arrangement of regulatory elements. In some cases, the promoters can be synthetic promoters. In some cases, the promoters in an expression cassette may be one or more temporally controlled promoters (for instance, TLR1, GND2, RPL40B promoters), one or more catabolite de-repression promoters (for instance, FMD promoters), one or more housekeeping promoters (for instance, GCW14 promoter), one or more stress-induced promoters (HAC1 promoter), one or more peroxisome biogenesis promoters (PEX11, PMP20, PMP47 promoters), one or more carbon/metabolite induced promoters (for instance, AOX1, DAS1, DAS2, FLD1 FDH1, FGH1, TMA10, RGIP, MH2 promoters or promoters P1-P7 from Table 1) or other such promoters.

The promoter for an expression cassette can be an inducible promoter. Inducible promoters include promoters that express under conditions where the inducer, such as a small molecule, protein, peptide, temperature, light or other environmental condition, induces expression and where absent the inducer, there is little or no expression. In some embodiments, an expression cassette includes an alcohol inducible promoter, such as a methanol inducible promoter. In some embodiments, such as where two or more different expression cassettes are employed in the systems and methods herein, each expression cassette can employ a different inducible promoter. In some embodiments, such as where two or more different expression cassettes are employed in the systems and methods herein, each expression cassette can employ the same inducible promoter. In some embodiments, the promoters of the first and the second expression cassettes are different promoter sequences, but are all inducible by the same inducer, such as for example, all methanol inducible promoters. Illustrative methanol inducible promoters for use in Pichia include AOX1, AOX2, FDH, and sugar inducible promoters such as glucose-induced, glycerol-induced and rhamnose regulated promoters. Other examples of inducible promoters that can be included in the expression cassettes are described elsewhere in this disclosure.

An expression cassette can include a constitutive promoter which expresses absent the need for an inducer. Constitutive promoters for use herein can include those providing a spectrum of expression level from highly expression constitutive promoters, to those providing more moderate and lower expression levels. In some embodiments, such as where two or more different expression cassettes are employed in the systems and methods herein, an and a second type of expression cassette employ a different constitutive promoter. In some embodiments where two or more different expression cassettes are employed in the systems and methods herein, an expression cassette employs an inducible promoter, and a second expression cassette employs a constitutive promoter.

In some cases, the sequence identity of the promoter sequences may be a sequence having at least 80%, 90%, 95%, 96%, 97%, 98%, 99% or 99.5% homology to one of SEQ ID NOs set forth in Table 1. In some embodiments, the one or more promoters are selected from the group consisting of adh1+, alcohol dehydrogenase (ADH1, ADH2, ADH4), AHSB4m, AINV, alcohol oxidase I (AOX1), alcohol oxidase 2 (AOX2), dihydroxyacetone synthase (DAS), enolase (ENO, ENOI), formaldehyde dehydrogenase (FLD1), FMD, formate dehydrogenase (FMDH), G1, G6, GAA, GCW14, gdhA, glyceraldehyde-3-phosphate dehydrogenase (gpdA, GAP, GAPDH), phosphoglycerate mutase (GPM1), glycerol kinase (GUTI), HSP82, inv1+, isocitrate lyase (ICL1), acetohydroxy acid isomeroreductase (ILV5), KAR2, β-galactosidase (lac4). LEU2, melO, MET3, nmt1, NSP, pcbC, PET9, peroxin 8 (PEX8), phosphoglycerate kinase (PGK, PGK1), pho1, PHO5. PH089, phosphatidylinositol synthase (PIS1), PYK1, pyruvate kinase (pki1), RPS7, sorbitol dehydrogenase (SDH), 3-phosphoserine aminotransferase (SERI), SSA4, TEF, translation elongation factor 1 alpha (TEF1), THI11, homoserine kinase (THR1), tpi, TPS1, triose phosphate isomerase (TPI1), XRP2, YPT1, Tac promoter, AprE promoter, Pxut promoter, xylose-inducible promoters, RGIP, TMA10, MH2 promoters.

### Terminators

An expression cassette can include a terminator 3′ to the eventual site of a protein coding sequence. In some embodiments the terminator and promoter sequences are from the same gene source (e.g. a DAS promoter and a DAS terminator). In other embodiments, the promoter and terminator of an expression cassette are derived from different gene sources. In some embodiments, such as where two or more different expression cassettes are employed in the systems and methods disclosed herein, each expression cassette can employ a different terminator sequence. In some embodiments, such as where two or more different expression cassettes are employed in the systems and methods herein, each expression cassette can employ the same terminator.

In some cases, the sequence identity of the terminator sequences may be a sequence having at least 80%, 90%, 95%, 96%, 97%, 98%, 99% or 99.5% homology to either of SEQ ID NOs set forth in Table 2. In some embodiments, a terminator for an expression cassette is selected from the group consisting of adh1+, alcohol dehydrogenase (ADH1, ADH2, ADH4), AHSB4m, AINV, alcohol oxidase I (AOX1), alcohol oxidase 2 (AOX2), dihydroxyacetone synthase (DAS), enolase (ENO, ENOI), formaldehyde dehydrogenase (FLD1), FMD, formate dehydrogenase (FMDH), G1, G6, GAA, GCW14, gdhA, glyceraldehyde-3-phosphate dehydrogenase (gpdA, GAP, GAPDH), phosphoglycerate mutase (GPM1), glycerol kinase (GUTI), HSP82, inv1+, isocitrate lyase (ICL1), acetohydroxy acid isomeroreductase (ILV5), KAR2, β-galactosidase (lac4), LEU2, melO, MET3, nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SERI), SSA4, TEF, translation elongation factor 1 alpha (TEF1), THI11, homoserine kinase (THR1), tpi, TPS1, triose phosphate isomerase (TPI1), XRP2, and YPT1.

### Signal Secretion Sequences

In embodiments, the systems and methods provided herein are designed for production of a desired recombinant heterologous protein by stable integration of a plurality of expression cassettes in a host cell genome. In some cases, it is achieved by fusing a secretion signal in-frame to the coding region of the recombinant heterologous protein in the plurality of expression cassettes integrated into the host cell genome (once transformed with the coding construct). In some embodiments, a plurality of the expression cassettes can include a heterologous secretion signal (e.g., not derived natively from the heterologous protein to be expressed). In some embodiments, a plurality of the expression cassettes employed in the systems and methods herein, can include a heterologous secretion signal and lack any naturally occurring secretion signal.

In some embodiments, such as where two or more different expression cassettes are employed in the systems and methods disclosed herein, each expression cassette can employ a different secretion signal peptide sequence. In some embodiments, such as where two or more different expression cassettes are employed in the systems and methods disclosed herein, each expression cassette can employ the same secretion signal peptide sequence. Illustrative secretion signals include but are not limited to the mating factor alpha-factor pro sequence from Saccharomyces cerevisiae, an Ost1 signal sequence, hybrid Ost1-alpha-factor pro sequence, and synthetic signal sequences.

In some cases, the sequence identity of the signal peptides may be a sequence having at least 80%, 90%, 95%, 96%, 97%, 98%, 99% or 99.5% homology to either of SEQ ID NO set forth in Table 3. In any one of the preceding embodiments, the signal peptide may be selected from the group consisting of acid phosphatase, albumin, alkaline extracellular protease, α-mating factor, amylase, β-casein, carbohydrate binding module family 21-starch binding domain, carboxypeptidase Y, cellobiohydrolase I, dipeptidyl protease, glucoamylase, heat shock protein (e.g., bacterial Hsp70), hydrophobin, inulase, invertase, killer protein or killer toxin (e.g., 128 kDa pGKL killer protein, α-subunit of the K1 killer toxin (e.g., Kluyveromyces lactis, K1 toxin KILM1, K28 pre-pro-toxin, Pichia acaciae), leucine-rich artificial signal peptide CLY-L8, lysozyme, phytohemagglutinin, maltose binding protein, P-factor. Pichia pastoris Dse. Pichia pastoris Exg, Pichia pastoris Pir1, Pichia pastoris Scw, Pir4, and any combination thereof.

### Selectable Markers

In the systems and methods provided herein, an expression cassette for integration in the host cell can be designed lacking in selectable markers. In some other cases, an expression cassette for integration in the host cell can be designed for identification of a positive integrant using one or more selectable markers. In some cases, an expression cassette for integration in the host cell can include one or more antibiotic resistance genes, auxotrophic markers or a combination thereof. In some embodiments, such as where two or more different expression cassettes are employed in the systems and methods herein, each expression cassette can employ a different combination of selectable markers. In some embodiments, such as where two or more different expression cassettes are employed in the systems and methods herein, each expression cassette can employ the same combination of selectable markers. Illustrative selectable markers can include: an antibiotic resistance gene that encodes resistance to an antibiotic (e.g. zeocin, ampicillin, blasticidin, kanamycin, nourseothricin, chloramphenicol, tetracycline, triclosan, ganciclovir) or any combination thereof. Other examples of selectable markers can include an auxotrophic marker (e.g. ade1, arg4, his4, ura3, met2) or any combination thereof. In some cases, the auxotrophic marker may be a defective auxotrophic marker, e.g., leu2-d or a variant of leu2-d involved in leucine metabolism (Betancur et. al, 2017). In some cases, the sequence identity of the selectable markers may be a sequence having at least 80%, 90%, 95%, 96%, 97%, 98%, 99% or 99.5% homology to SEQ ID NOs set forth in Table No. 5.

### Illustrative Combinations of Genetic Elements for Expression Cassettes

The genetic elements of the expression cassette can be designed to be suitable for expression in the intended host cell organism. For example, the genetic elements in the plurality of expression cassettes can be codon-optimized for effective expression in the intended host cell organism.

An expression cassette can be constructed to comprise any combination of the genetic elements (e.g., promoters, terminators, signal sequence, selectable markers, etc.) In some examples, a host strain for the expression of a transgene coding sequence may be generated by transforming an expression cassette containing the pAOX1 promoter, the alpha mating factor secretion signal along, and/or a tAOX1 terminator along with a Ura3 selection marker. In some examples, the pDAS2 promoter may be combined with the alpha mating factor secretion signal and a tAOX1 terminator (no selectable marker) to generate a cassette. In some examples, an expression cassette can include the pPEX11 promoter and a tAOX1 terminator. In some examples, an expression cassette may include the pAOX1 promoter, an alpha mating factor secretion signal and a tAOX1 terminator along with a selection marker.

In some examples, a host strain for the expression of the target gene coding sequence may be generated by transforming an expression cassette containing a pAOX1 promoter, an alpha mating factor secretion signal, a tAOX1 terminator along with a selection marker. In some examples, a host strain for the expression of the heterologous coding sequence may be generated by transforming an expression cassette containing a pAOX1 promoter, an alpha mating factor secretion signal, a tAOX1 terminator along with a selection marker. In some examples, a host strain may be generated by transforming an expression cassette containing a pDAS2 promoter, an alpha mating factor secretion signal, a tAOX1 terminator along with a selection marker. In some examples, a host strain may be generated by transforming an expression cassette containing a pFLD1 promoter, an alpha mating factor secretion signal, a tAOX1 terminator along with a selection marker.

In some examples, a host strain for the expression of the coding sequence may be generated by transforming an expression cassette containing a pAOX1 promoter, an alpha mating factor secretion signal, a tAOX1 terminator along with a selection marker. In some examples, a host strain may be generated by transforming an expression cassette containing a pFDH1 promoter, an alpha mating factor secretion signal, a tAOX1 terminator along with a selection marker. In some examples, a host strain may be generated by transforming an expression cassette containing a pFLD1 promoter, an alpha mating factor secretion signal, a tAOX1 terminator along with a selection marker.

In some cases, the methods herein can include transformation with an expression cassette for the expression of a helper factor, such as one that promotes protein folding, protein stability, protein translation and/or that increase transcription from a promoter.

### Methods for Co-Transformation of Expression Cassettes

The methods herein employ co-transformation to generate the multiple expression cassettes into the genome. The expression cassettes (e.g., 1, 2, 3 or more different cassettes), which preferably lack a coding sequence of a heterologous gene, can be mixed together and transformed into a host cell. Alternate methods may employ pre-joined cassettes, whereby the DNA sequences for the multiple copies of a single cassette, or the DNA sequences for different expression cassettes are linked in vitro (e.g., in a single plasmid) prior to transformation. In some cases, one or more plasmids comprising a designated copy number of the heterologous expression constructs may be linearized and combined in a starting mixture of nucleic acids for a single transformation reaction into the host cell (e.g., Pichia). For example, plasmid 1 can contain 2 head-to-tail copies of a cassette with a promoter A, a secretion signal A, followed by a terminator A, while plasmid 2 is constructed with four head to tail copies of a cassette containing a promoter B, a secretion signal B, followed by a terminator B. Both plasmids can include a selection marker or cassette. In a combinatorial transformation, plasmids 1 and 2 are both linearized and combined in a starting mixture of nucleic acids for a single transformation reaction into a host cell such as a yeast cell and a transformation strain A is recovered.

In other cases, one or more plasmids comprising one or more copies of the expression cassettes lacking a coding sequence of a host cell may be sequentially transformed into the host cell. For example, strain A obtained from combinatorial transformation previously can be used as the starting material. Strain A can then be sequentially transformed with two plasmids (plasmid 3 and 4), each containing a signal sequence and a terminator sequence. Each plasmid can contain a unique combination of promoter and terminator. For example, plasmid 3 may comprise a promoter C and terminator A while Plasmid 4 contained a promoter D with terminator A. The backbone in plasmid 3 may comprise a Zeocin resistance gene. The backbone in plasmid 4 can comprise a Hygromycin resistance. First strain A can be transformed with plasmid 3 and a transformant strain B may be recovered by selection. Then strain B may be transformed with plasmid 4 and the final transformant strain C can be recovered by selection. In some cases, the plasmids may be integrated in the same genomic locus, or in the vicinity of the same genomic locus. In other cases, the plasmids may be integrated in different genomic loci.

In some cases, all plasmids—for instance plasmids 1, 2, 3 and 4 may lack a coding sequence of the gene of interest or a heterologous gene. In some cases, one or more plasmids comprising the one or more expression cassettes may lack a coding sequence of the gene of interest or a heterologous gene. In some cases, one or more plasmids comprising one or more expression cassettes may lack a coding sequence of the gene of interest or a heterologous gene and one or more plasmids comprise a coding sequence of the gene of interest or the heterologous gene.

Integration of Expression Cassettes into Host Cell Genome

In some embodiments, multiple expression cassettes are integrated into a single site in the genome of the host cell. In some embodiments, multiple expression cassettes are integrated within the vicinity of one another site in the genome of the host cell. In some cases, where two or more different expression cassettes are employed in the systems and methods disclosed herein, the integration sites of the expression cassettes can be located on the same chromosome. Alternatively, one or more expression cassettes may have integration sites on different chromosomes. For instance, a first and a second expression cassette can be located on the same chromosome. In some cases, additionally, a third or fourth expression cassette can be integrated in the genome of the engineered cell at an integration site different from that of the first cassette and second cassette. In some cases, additionally, a third expression cassette can be integrated in the genome of the engineered cell at the same integration site as that of the first cassette and second cassette. In some cases, the first, second, third and fourth expression cassettes may be introduced into the host cell as one plasmid or vector. Alternatively, they may be introduced into the cell in more than one plasmid or vectors. In some cases, where two or more different expression cassettes are employed in the systems and methods herein, the integration sites of the plurality of the expression cassettes can be located on homologous sites in different chromosomes of the host cell genome.

In some embodiments, the multiple expression cassettes are integrated in tandem at a genomic site of the host cell, where all the cassettes are in a single orientation (e.g., with reference to 5′ to 3′ orientation of the cassette). In some embodiments, the multiple expression cassettes are integrated into the genome of the host cell, in arrangements where one or more of the cassettes is in a different orientation as compared to other cassettes. In some cases, where two or more different expression cassettes are employed in the systems and methods herein, a first and a second expression cassette are integrated into the genome in opposite 5′ to 3′ orientations. In some cases, where two or more different expression cassettes are employed in the systems and methods herein, a first and a second expression cassette are integrated into the genome in the same 5′ to 3′ orientation.

In some cases, additionally, a third expression cassette can integrate in the genome of the engineered cell at an integration site in a 5′ to 3′ orientation different from that of the first cassette, the second cassette, or both the first and the second cassette. In some cases, additionally, a third expression cassette can be integrated in the genome of the engineered cell in the same 5′ to 3′ orientation as that of the first cassette, the second cassette or both the first and the second cassette. In some cases, the orientation of the integrated expression constructs may be different in the host cell as compared to their orientation prior to their introduction to the host cell. For instance, a first, a second and a third expression cassette may be sequential in a plasmid but once they are integrated into the host cell they may have a different orientation, order or location.

In some embodiments, multiple expression cassettes may be ectopically integrated by non-homologous recombination in a single genomic locus of the host cell genome. In some embodiments, multiple expression cassettes may be ectopically integrated by non-homologous recombination in the vicinity of or at the same genomic locus in the host cell genome. In some embodiments, multiple expression cassettes may be ectopically integrated by non-homologous recombination on the same chromosome in the host cell genome. In some embodiments, multiple expression cassettes may be ectopically integrated by non-homologous recombination on different chromosomes in the host cell genome.

In some cases, multiple expression cassettes can be integrated into the genome of the host cell, by non-homologous recombination methods. In some cases, where two or more different expression cassettes are employed in the systems and methods herein, each expression cassette of the plurality of the expression cassettes can be integrated by non-homologous recombination. In some cases, where two or more different expression cassettes are employed in the systems and methods herein, at least one of the expression cassettes in the plurality of expression cassettes can be integrated by non-homologous recombination.

In some cases, where two different expression cassettes are employed in the systems and methods herein, the first and the second expression cassettes can be both integrated by non-homologous recombination. In some cases, multiple expression cassettes can be integrated into the host cell genome by homologous recombination. In some cases, where two or more different expression cassettes are employed in the systems and methods herein, a first expression cassette can be integrated by non-homologous recombination and a second expression cassette can be integrated by homologous recombination. In some cases, additionally, a third expression cassette can integrate in the genome of the engineered cell by a recombination method different from that of the first cassette, the second cassette or both the first and the second cassette. In some cases, additionally, a third expression cassette can be integrated in the genome of the engineered cell by the same recombination method as the first cassette, the second cassette or both the first and the second expression cassette.

In some cases, there is insubstantial sequence homology between a sequence in the one or more expression cassettes and a corresponding sequence in the host cell genome resulting in random integration of the one or more expression cassettes. For instance, where two or more different expression cassettes are employed in the systems and methods herein, the genomic locus of integration in the host cell does not share sequence homology with a first promoter, second promoter, first gene, second gene, first signal sequence, second signal sequence, first selective marker or second selective marker.

In some cases, there is sequence homology between a sequence in the host cell genome and one or more sequences with an expression cassette. In some cases, the sequence homology resides at or in part at a sequence at the 5′ and 3′ ends of a linearized expression cassette. In some cases, where two different expression cassettes are employed in the systems and methods herein, and where the first expression cassette and second expression cassette are linear molecules, the first expression cassette or the second expression cassette can comprise homology at the 5′ end with the host cell genome locus. For instance, the sequence homology between a sequence in the genomic locus of integration and a sequence at the 5′ sequence or the 3′ sequences of an expression cassette may be at least 5 bp, at least 10 bp, at least 20 bp, at least 30 bp, at least 40 bp, at least 60 bp, at least 80 bp, at least 100 bp, at least 120 bp, at least 150 bp, at least 180 bp, at least 200 bp, at least 250 bp, at least 300 bp, at least 350 bp, at least 400 bp, at least 450 bp, at least 500 bp, at least 600 bp, at least 700 bp long, at least 800 bp long, at least 900 bp long or at least 1000 bp long. In some cases, the sequence homology between a sequence in the genomic locus of integration and a sequence at the 5′ sequence or the 3′ sequences of an expression cassette may be at most 10 bp, at most 20 bp, at most 30 bp, at most 40 bp, at most 60 bp, at most 80 bp, at most 100 bp, at most 120 bp, at most 150 bp, at most 180 bp, at most 200 bp, at most 250 bp, at most 300 bp, at most 350 bp, at most 400 bp, at most 450 bp, at most 500 bp, at most 600 bp, at most 700 bp long, at most 800 bp long, at most 900 bp long or at most 1000 bp long.

In some examples, the sequence homology between a sequence in the genomic locus of integration and a sequence at the 5′ sequence or the 3′ sequences of an expression cassette may be at least 50 bp, at least 100 bp, at least 200 bp, at least 300 bp, at least 400 bp, at least 600 bp, at least 800 bp, at least 1000 bp, at least 1200 bp, at least 1500 bp, at least 1800 bp, at least 2000 bp, at least 2500 bp, at least 3000 bp, at least 3500 bp, at least 4000 bp, at least 4500 bp, at least 5000 bp, at least 6000 bp, at least 7000 bp long, at least 8000 bp long, at least 9000 bp long or at least 10,000 bp long. In some cases, the sequence homology between a sequence in the genomic locus of integration and a sequence at the 5′ sequence or the 3′ sequences of an expression cassette may be at most 100 bp, at most 200 bp, at most 300 bp, at most 400 bp, at most 600 bp, at most 800 bp, at most 100) bp, at most 1200 bp, at most 1500 bp, at most 1800 bp, at most 2000 bp, at most 2500 bp, at most 3000 bp, at most 3500 bp, at most 4000 bp, at most 4500 bp, at most 5000 bp, at most 6000 bp, at most 7000 bp long, at most 8000 bp long, at most 9000 bp long or at most 10,000 bp long.

In some cases, the expression cassettes may be integrated by homologous recombination by relying on the sequence homology between a sequence in the expression cassette and a corresponding sequence in the host cell genome. In some cases, the homologous recombination may rely on the sequence homology between a promoter sequence in a first expression cassette and the genomic promoter sequence. For instance, the homologous recombination may rely on the sequence homology between an AOX1 promoter in the expression cassette and the genomic AOX1 sequence. In some cases, the homologous recombination may rely on the sequence homology between a secretion signal sequence in a first expression cassette and the secretion signal sequence in the host cell genome cell. In some cases, the homologous recombination may rely on the sequence homology between a selective marker sequence in a first expression cassette and the genomic sequence.

In some cases, the host cell is first transformed with a first plurality of expression cassettes. Later, the host cell is transformed with a second plurality of expression cassettes. The second expression cassettes may be designed to differ from the first plurality of expression cassettes. For example, transcriptional elements, e.g., promoters, signal sequences, and terminator sequences, of the second plurality may differ, at least in part, the transcriptional elements of the first plurality. Without wishing to be bound by theory, if there is substantial homology in expression cassettes present in the genome (as a result of the first transformation with the first plurality of expression cassettes), when a second plurality of expression cassettes are later transformed, expression cassettes may displace the expression cassettes present in the genome via homologous recombination. In the most extreme case, rather than increasing the number of expression cassettes by a later transformation, after the second transformation final number of expression cassettes integrated into the genome may be unchanged relative to the first transformation

After a host cell is first transformed with a first plurality of expression cassettes and a first plurality of coding constructs, those host cells having desirable properties (e.g., viability, proliferation rate, and/or expression of coding constructs) may be selected for transformation with a second plurality of expression cassettes.

### Heterologous Gene

The systems and methods herein are particularly useful for producing heterologous proteins in engineered host cell. One of the various advantages of the methods, engineered host cells, and kits described herein is the production of one or more heterologous proteins with minimal cloning. The gene of interest sequence or the heterologous gene sequence may be transformed into the host cell comprising one or more expression cassettes. The one or more expression cassettes, as described herein may comprise the transcriptional elements required for the expression of the heterologous gene and therefore, a single transformation may reduce number of transformation rounds required using other conventionally used techniques. This may also reduce the time and cost to isolate a high titer strain. Additionally, the same host cell background comprising one or more integrated expression cassettes may be used to express more than one heterologous genes concurrently or independently of each other. Another advantage of the methods, engineered host cells, and kits described herein is the ability to express larger gene sequences which are difficult to transfect using conventional techniques such as plasmids.

In some cases, one or more coding constructs comprising a sequence for a heterologous gene may be introduced into the engineered host cells. Introduction of coding constructs may be done using conventionally used techniques such as transformation, transfection, etc. In some cases, 1, 2, 3, 4, 5, 6, 7, 8, 9, 10, 12, 14, 16, 18 or 20 different or unique coding constructs may be introduced into the host cells. The number of unique coding constructs may depend on the number of unique expression constructs integrated into the host cell. In some cases, at least 2 different coding constructs are introduced into the host cells. In some cases, at least 3 different coding constructs are introduced into the host cells. In some cases, at least 4 different coding constructs are introduced into the host cells. In some cases, at least 5 different coding constructs are introduced into the host cells. In some cases, at least 6 different coding constructs are introduced into the host cells. In some cases, at least 8 different coding constructs are introduced into the host cells. In some cases, at most 2 different coding constructs are introduced into the host cells. In some cases, at most 3 different coding constructs are introduced into the host cells. In some cases, at most 4 different coding constructs are introduced into the host cells. In some cases, at most 5 different coding constructs are introduced into the host cells. In some cases, at most 6 different coding constructs are introduced into the host cells. In some cases, at most 8 different coding constructs are introduced into the host cells. In some cases, at most 10 different coding constructs are introduced into the host cells. In some cases, at most 12 different coding constructs are introduced into the host cells. In some cases, at most 16 different coding constructs are introduced into the host cells.

The one or more coding constructs may be introduced into the host cell in a single round of transformation. In some cases, the coding constructs may be introduced into the host cell in more than one round of transformation.

In some embodiments, the one or more coding constructs may be vector-less. A vector-less coding construct as described herein may refer to a coding construct which is lacking an autonomously replicating sequence, an origin of replication or any other replicating or backbone elements found in plasmids. Alternatively, in some cases, the one or more coding constructs comprising a sequence for a heterologous gene may be comprised in a plasmid or a vector.

One or more coding constructs comprising a heterologous gene sequence may lack regulatory or transcriptional elements. Alternatively, a coding construct may comprise one or more regulatory or transcriptional elements. For instance, a coding construct may comprise the sequence of a heterologous gene but may lack a promoter sequence linked operably to the gene sequence. In some examples, the coding construct may comprise a signal sequence but may lack a promoter sequence. In some examples, the coding construct may comprise a signal sequence and/or a terminator sequence but may lack a promoter sequence.

One or more coding constructs to be transformed into the host cells may be in the form of linear DNA. One or more coding constructs to be transformed into the host cells may be in the form of double stranded DNA. One or more coding constructs to be transformed into the host cells may be single stranded DNA. A combination of coding constructs may be transformed into a single host cell, wherein the coding constructs may include a single stranded DNA construct, a double stranded DNA construct and/or a vector or plasmid comprising the coding construct.

The coding constructs described herein may comprise one or more recognition zones. The recognition zone sequences may be added to the coding constructs to provide homology to the expression constructs integrated in the host cell. The recognition zones in the coding constructs may direct each coding construct to a specific expression cassette and aid the homologous recombination of the coding construct into the host cell. Coding constructs may comprise more than one recognition zone. Recognition zones may be at the 5′ and/or 3′ ends of the coding sequence of the heterologous gene. In some cases, the recognition zone may comprise a signal sequence, a promoter sequence and/or a terminator sequence. In some cases, the recognition zone may comprise at least a partial signal sequence, promoter sequence and/or terminator sequence. In one example, the recognition zone at the 5′ of the coding sequence may comprise a signal sequence or promoter sequence and the recognition zone at the 3′ of the coding sequence may comprise a terminator sequence.

In some cases, a recognition sequence in a coding construct may comprise 40 nucleotides to 600 nucleotides. In some cases, a recognition sequence in a coding construct may comprise at least 40 nucleotides. In some cases, a recognition sequence in a coding construct may comprise at most 600 nucleotides. In some cases, a recognition sequence in a coding construct may comprise 40 nucleotides to 80 nucleotides, 40 nucleotides to 100 nucleotides, 40 nucleotides to 140 nucleotides, 40 nucleotides to 180 nucleotides, 40 nucleotides to 200 nucleotides, 40 nucleotides to 250 nucleotides, 40 nucleotides to 300 nucleotides, 40 nucleotides to 350 nucleotides, 40 nucleotides to 400 nucleotides, 40 nucleotides to 500 nucleotides, 40 nucleotides to 600 nucleotides, 80 nucleotides to 100 nucleotides, 80 nucleotides to 140 nucleotides, 80 nucleotides to 180 nucleotides, 80 nucleotides to 200 nucleotides, 80 nucleotides to 250 nucleotides, 80 nucleotides to 300 nucleotides, 80 nucleotides to 350 nucleotides, 80 nucleotides to 400 nucleotides, 80 nucleotides to 500 nucleotides, 80 nucleotides to 600 nucleotides, 100 nucleotides to 140 nucleotides, 100 nucleotides to 180 nucleotides, 100 nucleotides to 200 nucleotides, 100 nucleotides to 250 nucleotides, 100 nucleotides to 300 nucleotides, 100 nucleotides to 350 nucleotides, 100 nucleotides to 400 nucleotides, 100 nucleotides to 500 nucleotides, 100 nucleotides to 600 nucleotides, 140 nucleotides to 180 nucleotides, 140 nucleotides to 200 nucleotides, 140 nucleotides to 250 nucleotides, 140 nucleotides to 300 nucleotides, 140 nucleotides to 350 nucleotides, 140 nucleotides to 400 nucleotides, 140 nucleotides to 500 nucleotides, 140 nucleotides to 600 nucleotides, 180 nucleotides to 200 nucleotides, 180 nucleotides to 250 nucleotides, 180 nucleotides to 300 nucleotides, 180 nucleotides to 350 nucleotides, 180 nucleotides to 400 nucleotides, 180 nucleotides to 500 nucleotides, 180 nucleotides to 600 nucleotides, 200 nucleotides to 250 nucleotides, 200 nucleotides to 300 nucleotides, 200 nucleotides to 350 nucleotides, 200 nucleotides to 400 nucleotides, 200 nucleotides to 500 nucleotides, 200 nucleotides to 600 nucleotides, 250 nucleotides to 300 nucleotides, 250 nucleotides to 350 nucleotides, 250 nucleotides to 400 nucleotides, 250 nucleotides to 500 nucleotides, 250 nucleotides to 600 nucleotides, 300 nucleotides to 350 nucleotides, 300 nucleotides to 400 nucleotides, 300 nucleotides to 500 nucleotides, 300 nucleotides to 600 nucleotides, 350 nucleotides to 400 nucleotides, 350 nucleotides to 500 nucleotides, 350 nucleotides to 600 nucleotides, 400 nucleotides to 500 nucleotides, 400 nucleotides to 600 nucleotides, or 500 nucleotides to 600 nucleotides. In some cases, a recognition sequence in a coding construct may comprise 40 nucleotides, 80 nucleotides, 100 nucleotides, 140 nucleotides, 180 nucleotides, 200 nucleotides, 250 nucleotides, 300 nucleotides, 350 nucleotides, 400 nucleotides, 500 nucleotides, or 600 nucleotides. In some cases, a recognition sequence in a coding construct may comprise at least 40 nucleotides, 80 nucleotides, 100 nucleotides, 140 nucleotides, 180 nucleotides, 200 nucleotides, 250 nucleotides, 300 nucleotides, 350 nucleotides, 400 nucleotides, or 500 nucleotides. In some cases, a recognition sequence in a coding construct may comprise at most 80 nucleotides, 100 nucleotides, 140 nucleotides, 180 nucleotides, 200 nucleotides, 250 nucleotides, 300 nucleotides, 350 nucleotides, 400 nucleotides, 500 nucleotides, or 600 nucleotides.

In some cases, a recognition sequence in a coding construct may comprise 500 nucleotides to 3,000 nucleotides. In some cases, a recognition sequence in a coding construct may comprise at least 500 nucleotides. In some cases, a recognition sequence in a coding construct may comprise at most 3,000 nucleotides. In some cases, a recognition sequence in a coding construct may comprise 500 nucleotides to 1,000 nucleotides, 500 nucleotides to 1,500 nucleotides, 500 nucleotides to 2,000 nucleotides, 500 nucleotides to 2,500 nucleotides, 500 nucleotides to 3,000 nucleotides, 1,000 nucleotides to 1,500 nucleotides, 1,000 nucleotides to 2,000 nucleotides, 1,000 nucleotides to 2,500 nucleotides, 1,000 nucleotides to 3,000 nucleotides, 1,500 nucleotides to 2,000 nucleotides, 1,500 nucleotides to 2,500 nucleotides, 1,500 nucleotides to 3,000 nucleotides, 2,500 nucleotides to 2,500 nucleotides, 2,000 nucleotides to 3,000 nucleotides, or 2,500 nucleotides to 3,000 nucleotides. In some cases, a recognition sequence in a coding construct may comprise about 500 nucleotides, 1,000 nucleotides, 1,500 nucleotides, 2,000 nucleotides, 2,500 nucleotides, or 3,000 nucleotides. In some cases, a recognition sequence in a coding construct may comprise at least 500 nucleotides, 1,000 nucleotides, 1,500 nucleotides, 2,000 nucleotides or 2,500 nucleotides. In some cases, a recognition sequence in a coding construct may comprise at most 1,000 nucleotides, 1,500 nucleotides, 2,000 nucleotides, 2,500 nucleotides, or 3,000 nucleotides.

The coding constructs described herein may comprise a promoter sequence operably linked to the heterologous gene sequence. The promoter sequence may be a partial promoter sequence. The promoter sequence may be a full-length promoter sequence. One or more coding constructs to be transformed into the host cell may comprise different promoters. Alternatively, one or more coding constructs to be transformed into the host cell may comprise the same promoters. For instance, more than one coding constructs may be transformed into a host cell wherein a first coding construct has a promoter sequence different than the promoter sequence in a second or third construct. A promoter sequence in a coding construct may be homologous to one or more promoters in one or more expression cassettes integrated into the host cell. Each of the one or more coding constructs may comprise different promoter sequences. In some cases, the different promoter sequences in the one or more coding constructs are all homologous to promoter sequences in the expression constructs integrated into the host cell.

The coding constructs described herein may comprise a signal sequence operably linked to the heterologous gene sequence. The signal sequence may be a partial signal sequence. The signal sequence may be a full-length signal sequence. One or more coding constructs to be transformed into the host cell may comprise different signal sequences. Alternatively, one or more coding constructs to be transformed into the host cell may comprise the same signal sequences. For instance, more than one coding constructs may be transformed into a host cell wherein a first coding construct has a signal sequence different than the signal sequence in a second or third construct. A signal sequence in a coding construct may be homologous to one or more signals in one or more expression cassettes integrated into the host cell. Each of the one or more coding constructs may comprise different signal sequences. In some cases, the different signal sequences in the one or more coding constructs are all homologous to signal sequences in the expression constructs integrated into the host cell.

The coding constructs described herein may comprise a terminator sequence operably linked to the heterologous gene sequence. The terminator sequence may be a partial terminator sequence. The terminator sequence may be a full-length terminator sequence. One or more coding constructs to be transformed into the host cell may comprise different terminator sequences. Alternatively, one or more coding constructs to be transformed into the host cell may comprise the same terminator sequences. For instance, more than one coding constructs may be transformed into a host cell wherein a first coding construct has a terminator sequence different than the terminator sequence in a second or third construct. A terminator sequence in a coding construct may be homologous to one or more terminators in one or more expression cassettes integrated into the host cell. Each of the one or more coding constructs may comprise different terminator sequences. In some cases, the different terminator sequences in the one or more coding constructs are all homologous to terminator sequences in the expression constructs integrated into the host cell.

The one or more coding constructs described herein may be different from each other. The one or more coding constructs may each comprise the coding sequence of the same heterologous gene. Alternatively, the one or more coding constructs may comprise coding sequences for more than one heterologous gene. In some cases, the coding constructs transformed into the host cell comprise coding sequences for at least 2 heterologous genes. In some cases, the coding constructs transformed into the host cell comprise coding sequences for at least 3 heterologous genes. In some cases, the coding constructs transformed into the host cell comprise coding sequences for at least 4 heterologous genes. In one example, a pool of coding constructs may comprise a coding construct with a coding sequence of a nutritional protein and another coding construct with a coding sequence of a helper protein. In such examples, the two different coding constructs may be directed for integration in different expression constructs. For instance, a coding construct A may comprise a coding sequence for protein 1 and comprises a recognition zone with homology to promoter A. A coding construct B may comprise a coding sequence for helper protein B and comprises a recognition zone with homology to promoter B.

In some cases, the host cell integration can include 5 to 120 copies of a gene sequence encoding a first recombinant protein. In some cases, the host cell integration can include at least 5 copies of a gene sequence encoding a first recombinant protein. In some cases, the host cell integration can include at most 120 copies of a gene sequence encoding a first recombinant protein. In some cases, the host cell integration can include 5 to 10, 5 to 15, 5 to 20, 5 to 30, 5 to 50, 5 to 70, 5 to 90, 5 to 100, 5 to 120, 10 to 15, 10 to 20, 10 to 30, 10 to 50, 10 to 70, 10 to 90, 10 to 100, 10 to 120, 15 to 20, 15 to 30, 15 to 50, 15 to 70, 15 to 90, 15 to 100, 15 to 120, 20 to 30, 20 to 50, 20 to 70, 20 to 90, 20 to 100, 20 to 120, 30 to 50, 30 to 70, 30 to 90, 30 to 100, 30 to 120, 50 to 70, 50 to 90, 50 to 100, 50 to 120, 70 to 90, 70 to 100, 70 to 120, 90 to 100, 90 to 120, or 100 to 120 copies of a gene sequence encoding a first recombinant protein. In some cases, the host cell integration can include 5, 10, 15, 20, 30, 50, 70, 90, 100, or 120 copies of a gene sequence encoding a first recombinant protein. In some cases, the host cell integration can include at least 5, 10, 15, 20, 30, 50, 70, 90, 100, or 120 copies of a gene sequence encoding a first recombinant protein. In some cases, the host cell integration can include at most 5, 10, 15, 20, 30, 50, 70, 90, 100, or 120 copies of a gene sequence encoding a first recombinant protein.

In some cases, an engineered host cell can integrate one or more copies of a gene sequence encoding a first recombinant protein, one or more copies of a gene sequence encoding a second recombinant protein, and optionally one or more copies of a transgene encoding a third recombinant protein. In some cases, the host cell integration can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a transgene encoding a first recombinant protein and at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19 or at least 20 copies of a transgene encoding a second recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a third recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a fourth recombinant protein. Additionally, the host cell can include at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19 or at most 20 copies of a transgene encoding a fifth recombinant protein.

In some embodiments, the integration of the coding constructs into the expression constructs in the genome may not require any additional enzymes such as nucleases or endonucleases. For instance, the integration of the coding constructs may be performed with any nuclease (or endonuclease) target sequences and site-specific nucleases. The nuclease can be any nuclease such as a homing endonuclease, zinc-finger nuclease or TAL-effector nuclease. Without wishing to be bound by theory, double stranded breaks by nucleases and endonucleases may lead to a reduction in viability and stability of the host cell. To avoid this reduction in viability and stability, the methods described herein may depend on homologous recombination mediated integration of the expression and coding constructs. In some embodiments, the integration of coding constructs is done only using homologous recombination. In some embodiments, the host cells comprising the expression cassettes and/or coding constructs described herein have a higher viability as compared to a control strain where the coding constructs are integrated using nucleases or endonucleases. In some embodiments, the host cells comprising the expression cassettes and/or coding constructs described herein have a higher titer of the protein of interest as compared to a control strain where the coding constructs are integrated using nucleases or endonucleases. In some embodiments, the host cells comprising the expression cassettes and/or coding constructs described herein have a higher copy number of the gene of interest as compared to a control strain where the coding constructs are integrated using nucleases or endonucleases.

In some embodiments, coding constructs greater than 1 kb can be integrated into the genomes of host cells without the need for a nuclease, endonuclease or a nuclease type enzyme. In some embodiments, coding constructs greater than 1 kb, 2 kb, 3 kb, 4 kb, 5 kb, 7 kb, 10 kb, 12 kb, 15 kb, 17 kb, 20 kb, 25 kb or 30 kb can be integrated into the genomes of host cells without the need for a nuclease or a nuclease type enzyme.

In some cases, the recombinant heterologous proteins encoded by the coding constructs can be animal-derived proteins. In some cases, the animal-derived proteins are food-related proteins. In some cases, the animal-derived proteins can be egg-related proteins. Examples of egg-related proteins or egg-white proteins include for example, ovomucoid, ovalbumin, lysozyme, ovotransferrin, ovomucin, ovoglobulin G2, ovoglobulin G3 and any combination thereof. Additional egg-related proteins for production include ovoinhibitor, ovoglycoprotein, flavoprotein, ovomacroglobulin, ovostatin, cystatin, avidin, ovalbumin related protein X, ovalbumin related protein Y and any combination thereof.

In some cases, the recombinant heterologous proteins encoded by the first or the second expression cassettes can be plant-based food proteins. In some cases, the one or more plant-based proteins may include, but are not limited to: pea protein; garbanzo (chickpea) protein; fava bean protein; soy protein; rice protein; mung bean protein; potato protein; hemp protein; or any combinations thereof. Plant-based proteins may include, for example, soy protein, pea protein, canola protein, or other plant proteins that are commercially-relevant, including wheat and fractionated wheat proteins, corn and it fractions including zein, rice, oat, potato, peanut, green pea powder, green bean powder, and any proteins derived from beans, lentils, and pulses. In particular embodiments, the pea proteins can be derived from yellow peas, such as Canadian yellow peas.

### Host Cell Engineering

A host cell can be modified in addition to and separately from integrating the expression cassettes. Such modification can be performed prior to or subsequent to transformation with the expression cassettes. In some instances, the modification contributes to the growth features and/or expression features of the host cell and thereby assists in the production of high protein tiers under fermentation conditions.

In some embodiments, the modification alters the host cell response to an inducer. For example, one such modification is a mutS modification which alters the growth characteristics of the host cell (e.g., Pichia) to methanol. In some embodiments, a mutS host is used as the host cell for further transformation and integration of expression cassettes where one or more of the cassettes includes a promoter inducible by methanol. In some embodiments, the modification includes the expression of one or more factors that increase the amount of, accumulation of or the production of an active form of the protein encoded by the expression cassettes. Such modifications can include the expression of one or more helper factors (such as transcription factors, chaperones and other proteins that participate in protein folding), post-transcriptional modification enzymes (e.g., phosphorylases, phosphatases, glycosylation and deglycosylation enzymes).

In some embodiments, a host cell (e.g., a Pichia cell) may be engineered to display increased non-homologous recombination (NHEJ) as compared to homologous recombination. For instance, in some cases, a host cell (e.g., a Pichia cell) may be engineered to overexpress a gene that is involved in non-homologous recombination activity of the cell (i.e., one or more genes that encode proteins that drives the NHEJ pathway or contribute to NHEJ). Examples of NHEJ pathway genes for Pichia include, but are not limited to, YKU70, YKU 80, DNL4, Rad50, Rad 27, MRE1 1, and POL4. The names of genes may be different for different host cells. The increase in NHEJ activity can be a reduction in homologous recombination of at least 20%, 30%, 40%, 50%, 60%, 70%, 80%, 90%, 100% or any percent reduction in between these percentages, as compared to a host cell that does not overexpress a gene controlling NHEJ in the cell, for example, the YKU70 gene locus of a Pichia cell.

To alleviate the depletion of intracellular amino acids concentrations that may occur due to high recombinant protein production, the host cells may be engineered to improve the supply of amino acids and therefore protein production. In some embodiments, overexpression of GCN4 (encoding a general transcriptional activator of amino acid biosynthesis, direct overexpression of metabolic enzymes in the anabolism of serine, isoleucine, alanine and aromatic amino acids, or of a fungal carboxylesterase can be used to optimize the synthesis pathways of amino acids by tuning enzyme abundance or their kinetics.

To overcome the constraints of energetic inefficiencies that may occur due to high recombinant protein production, the host cells can be optimized for an improved supply of precursors involved in cellular redox and energy efficiency. In some embodiments, strategies may include deletion of genes diverting carbon towards fermentative pathways, overexpression of malate dehydrogenase, which could increase the supply of mitochondrial NADH, or overexpression of enzymes in the oxidative part of the PPP (e.g., NADH oxidase) causing an increased supply of NADPH and precursors and thereby higher titer protein production.

Undesired proteolysis of heterologous proteins expressed in host cells does not only lower the product yield or biological activity but can also complicate downstream processing of the intact product as the degradation products will have similar physicochemical and affinity properties. In order to alleviate the proteolysis problem, protease-deficient host cells strains lacking proteases can be used. Examples of proteases include PEP4, carboxypeptidase Y (PRC1) and proteinase B (PRB1). Examples of such protease-deficient strains include SMD1163 (Δhis4 Δpep4 Δprb1), SMD1165 (Δhis4 Δprb1) and SMD1168 (Δhis4 Δpep4).

High recombinant protein production can induce secretory bottlenecks in the form of inappropriate mRNA structure, incomplete protein folding or protein translocation to the ER. Host cells can be engineered to overcome potential secretory bottleneck by the overexpression of folding helper proteins such as iP/Kar2p, DnaJ, PDI, PPIs and Ero1p or, alternatively, overexpression of HAC1, a transcriptional regulator of the UPR pathway genes.

In some cases, heterologous protein production in host cells may be accompanied by high mannose glycan structures, affecting serum half-life or triggering of allergic reactions in the human body. To alleviate this problem, the host cells may be further engineered to include the knockout of protein-O-mannosyltransferases (PMTs) or the yeast Golgi protein α-1,6-mannosyltransferase encoded by OCH1. In other cases, the host cells may be engineered to express a Trichoderma reesei α-1,2-mannosidase or one of several glycosyltransferases and glycosidases (e.g., β-1,2-N-acetylglucosaminyl-transferase 1, uridine 5′-diphosphate (UDP)-GlcNAc transporter, mouse mannosidase MnsIA catalytic domain fused to the N-terminal localization peptide of the ER protein Sec12 from S. cerevisiae, human GlcNAc transferase GnTI fused to the leader sequence from the S. cerevisiae Golgi protein Mnn9, overexpression of Drosophila melanogaster mannosidase II (ManII) or rat GlcNAc transferase GnTII, overexpression of Schizosaccharomyces pombe galactose epimerase or human β-1,4 galactosyl transferase) carrying proper targeting signals may be used. In some cases, genes involved in sialic acid synthesis, transport and transfer may be co-expressed, for example, human UDP-N-acetylglucosamine-2-epimerase/N-acetylmannosamine kinase (GNE), human N-acetylneuraminate-9-phosphate synthase (SPS), human CMP-sialic acid synthase (CSS), mouse CMP-sialic acid transporter (CST), to achieve optimal sialyated N-glycans.

In some cases, the recombinant host cell may be a methanotroph. Among methanotrophs, Komagataella pastoris and Komagataella phaffii are preferable (also known as Pichia pastoris). Examples of strains in the Pichia genus include Pichia pastoris strains. Examples can include NRRL Y-11430, BG08, BG10, NRRL Y-11430 GS115 (NRRL Y-15851), GS190 (NRRL Y-18014), PPF1 (NRRL Y 18017), PPY1200H, YGC4, and strains derived therefrom. Other examples of P. pastoris strains that may be used as host cells include but are not limited to CBS7435 (NRRL Y-11430). CBS704 (DSMZ 70382) or derivatives thereof. Other examples of methanol-utilizing yeast include yeasts belonging to Ogataea (Ogataea polymorpha). Candida (Candida boidinii). Torulopsis (Torulopsis) or Komagataella.

Further examples of suitable host cell organisms include but are not limited to eukaryotic cells such as: Arxula spp., Arxula adeninivorans. Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa. Penicillium spp., Penicillium camemberti, Penicillium canescens, Penicillium chrysogenum, Penicillium (Talaromyces) emersoni, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei. Trichoderma vireus, Aspergillus oryzae. Bacillus subtilis, Escherichia coli, Myceliophthora thermophila, Neurospora crassa, Pichia pastoris, Pichia Pastoris “MutS” strain (Graz University of Technology (CBS7435Mu1S) or Biogrammatics (BGi1)), Komagatella phaffii, and Komagatella pastoris.

In some cases, a bacterial host cell such as Lactococcus lactis, Bacillus subtilis or Escherichia coli may be used as the host cells. Other host cells include bacterial host such as, but not limited to, Lactococci sp., Lactococcus lactis, Bacillus subtilis, Bacillus amyloliquefaciens, Bacillus licheniformis and Bacillus megaterium, Brevibacillus choshinensis, Mycobacterium smegmatis, Rhodococcus erythropolis and Corynebacterium glutamicum, Lactobacilli sp., Lactobacillus fermentum. Lactobacillus casei, Lactobacillus acidophilus, Lactobacillus plantarum, Pseudomonas sp., Pseudomonas fluorescens.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Herein the term “sequence identity”, such as for the purpose of assessing percent complementarity, may be measured by any suitable alignment algorithm. In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby and comparing these sequences to a second nucleotide or amino acid sequence. Two or more sequences (polynucleotide or amino acid) can be compared by determining their “percent identity.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. Herein percentage sequence identity can refer to sequences and their alignment over the span of a query sequence. If one sequence is shorter than the other, than the percentage identity can be considered over the span of the shorter sequence. Herein “percentage coverage” can refer to the number of nucleotides or amino acids that align identically with the longer of the two sequences as a percentage of the number of nucleotides or amino acids in the longer sequence.

Herein one polynucleotide is referred to another polynucleotide as being a “copy” of the other if it has 100% sequence identity to another polynucleotide and is the same length. In some cases, one polynucleotide is referred to another polynucleotide as being a “copy” of the other if it has a different sequence, but the protein encoded by the two polynucleotides has the same amino acid sequence. Herein a polynucleotide is “different” from a set of polynucleotides if it is not a copy of any element of the set, or for all those elements of a sets that it is a copy, it contains chemical differences apart from its genetic or amino acid sequence that distinguishes it from that element.

Herein an “expression cassette” is any polynucleotide that contains a subsequence that codes for a transgene and can confer expression of that subsequence when contained in a host cell, and is heterologous to that host organism.

The section headings used herein are for organizational purposes only and are not to be construed as limiting the subject matter described.

TABLE 1
Promoter sequences

| SEQ ID | | ANNO- |
|---|---|---|
| NO. | SEQUENCE | TATION |
| 1 | AACATCCAAAGACGAAAGGTTGAATGAAACCTTTTTGCCATCCGACATCCACAGGTCCATTCTCACACATAAGTGCCAA | pAOX1 |
| | ACGCAACAGGAGGGGATACACTAGCAGCAGACCGTTGCAAACGCAGGACCTCCACTCCTCTTCTCCTCAACACCCACTT | |
| | TTGCCATCGAAAAACCAGCCCAGTTATTGGGCTTGATTGGAGCTCGCTCATTCCAATTCCTTCTATTAGGCTACTAACA | |
| | CCATGACTTTATTAGCCTGTCTATCCTGGCCCCCCTGGCGAGGTTCATGTTTGTTTATTTCCGAATGCAACAAGCTCCG | |
| | CATTACACCCGAACATCACTCCAGATGAGGGCTTTCTGAGTGTGGGGTCAAATAGTTTCATGTTCCCCAAATGGCCCAA | |
| | AACTGACAGTTTAAACGCTGTCTTGGAACCTAATATGACAAAAGCGTGATCTCATCCAAGATGAACTAAGTTTGGTTCG | |
| | TTGAAATGCTAACGGCCAGTTGGTCAAAAAGAAACTTCCAAAAGTCGGCATACCGTTTGTCTTGTTTGGTATTGATTGA | |
| | CGAATGCTCAAAAATAATCTCATTAATGCTTAGCGCAGTCTCTCTATCGCTTCTGAACCCCGGTGCACCTGTGCCGAAA | |
| | CGCAAATGGGGAAACACCCGCTTTTTGGATGATTATGCATTGTCTCCACATTGTATGCTTCCAAGATTCTGGTGGGAAT | |
| | ACTGCTGATAGCCTAACGTTCATGATCAAAATTTAACTGTTCTAACCCCTACTTGACAGCAATATATAAACAGAAGGAA | |
| | GCTGCCCTGTCTTAAACCTTTTTTTTTATCATCATTATTAGCTTACTTTCATAATTGCGACTGGTTCCAATTGACAAGC | |
| | TTTTGATTTTAACGACTTTTAACGACAACTTGAGAAGATCAAAAAACAACTAATTATTGAA | |
| 2 | AAATctgaGacaAcgatgaacctcccATgtagattccaccgccccAGttactTttttgggcaatccTgttgataagaTC | pDAS2 |
| | cattTtagagttgtttcAtgaaaggAttacAggCgttgaAGggtCAgagaGatgccagagAacagaCcaatTGgtAgtt | (1) |
| | tgctaaaGtggaCgtctggCaGgTGctctaTcGTgtTcTttaTtTaGGGCgtTaCacTTagtaGgattacgtaacaATt | |
| | TggcTtAacCttctaagttagAaagAAaccaagagggGtcctCtttaacgttCAgcAgtatctAaaacacaaAacCtgc | |
| | cCtcataAtacatcatTCtaTctgtcaagctGtgctaccccacagaaataccccCaaGagttAaagtgaaaaGAAAAGC | |
| | TAAATCTGTTAGacttcaccccataacaaacttGataGttCctgtagccaatgaaagtTaaccccattcaatgttccga | |
| | gatCtagtatGcttgcTcctataagGaacgaaggGttCcagcttccttaccccatCaaTGgaaatctCcTatttacccc | |
| | ccactggaAagatccgTccGaacgaacGgataatagaaaaaagaaattcggacaaaAtagaacacttATtTagccaatG | |
| | aaaTCcattTccagcatcTccttCaActgccgttccatcccCtttgttgagcTacaccatcgtCagccagtacCGaaTa | |
| | ggaaacttaaccgataTcttggagaaTtctaaTgcgcgaatgagtttagcctagatatccttagtgaagggttgttccg | |
| | atacttctccacattcagtcatTTCagatgggcagcAttgttatcatgaagaAacggaaacgggcaGTAagggttaacc | |
| | gccaaattatataaagacaacatgtccccagtttaaagttttttttcctattcttgtatcctgagtgaccgttgtgttt | |
| | aaAataacaagttcgttttaacttaagaccaaaaccagttacaacaaattattccccaactaaacactaaagttcactc | |
| | ttatcaaactatcaaacatcaaa | |
| 3 | gatctctgaGacaAcgatgaacctcccATgtagattccaccgccccAAttactGttttgggcaatccTgttgataagaC | pDAS2 |
| | GcattCtagagttgtttcAtgaaaggGttacGggTgttgaTTggtTTgagaTatgccagagGacagaTcaatCTgtGgt | (2) |
| | ttgctaaaCtggaAgtctggTaAgGActctaGcAAgtCcGttaCtCaAAAAgtCaTacCAagtaAgattacgtaacaCC | |
| | tGggcAtGacTttctaagttagCaagTCaccaagagggtcctAtttaacgttTGgcGgtatctGaaacacaaGacTtgc | |
| | cTAtcCCataGtacatcatATtaCctgtcaagctAtgctaccccacagaaataccccAaaagttGaagtgaaaaAATGA | |
| | AAATTACTGGTAacttcaccccataacaaacttAataAttTctgtagccaatgaaagtAaaccccattcaatgttccga | |
| | gatTtagtatActtgcCcctataagAaacgaaggAttTcagcttccttaccccatGaaCAgaaatctTcCatttacccc | |
| | ccactggaGagatccgCccAaacgaacAgataatagaaaaaagaaattcggacaaatagaacacttTCtCagccaatTa | |
| | aaGTcattccaTgcacTccCttTaGctgccgttccatccctttgttgagcAacaccatcgtTagccagtacGAaaGagg | |
| | aaacttaaccgataCcttggagaaAtctaagGcgcgaatgagtttagcctagatatccttagtgaagggttgttccgat | |
| | acttctccacattcagtcatagatgggcagcTttgttatcatgaagaGacggaaacgggcaTTAagggttaaccgccaa | |
| | attatataaagacaacatgtccccagtttaaagttttttttcctattcttgtatcCtgagtgaccgttgtgtttaaTat | |
| | aacaagttcgtittaacttaagacCaaaaccagttacaacasattatAAcccCTctaaacactaaagtTcactcttatc | |
| | aaactatcaaacatcaaa | |
| 4 | TGCTCCTATAAGGAACGAAGGGTTCCAGCTTCCTTACCCCATCAATGGAAATCTCCTATTTACCCCCCACTGGAAAGAT | pDAS2 |
| | CCGTCCGAACGAACGGATAATAGAAAAAAGAAATTCGGACAAAATAGAACACTTATTTAGCCAATGAAATCCATTTCCA | (3) |
| | GCATCTCCTTCAACTGCCGTTCCATCCCCTTTGTTGAGCTACACCATCGTCAGCCAGTACCGAATAGGAAACTTAACCG | |
| | ATATCTTGGAGACTTCTAATGCGCGAATGAGTTTAGCCTAGATATCCTTAGTGAAGGGTTGTTCCGATACTTCTCCACA | |
| | TTCAGTCATTTCAGATGGGCAGCATTGTTATCATGAAGAAACGGAAACGGGCAGTAAGGGTTAACCGCCAAATTATATA | |
| | AAGACAACATGTCCCCAGTTTAAAGTTTTTCTTTCCTATTCTTGTATCCTGAGTGACCGTTGTGTTTAAAATAACAAGT | |
| | TCGTTTTAACTTAAGACCAAAACCAGTTACAACAAATTATTCCCCAACTAAACACTAAAGTTCACTCTTATCAAACTAT | |
| | CAAACATCAAA | |
| 5 | cttccccatttcactgacagtttgtagaaatagggcaacaattgatgcaaatcgattttcaacgcattggttttgatag | pPEX11 |
| | cattgatgatcttggagctgtaaaagtccggctggataagctcaatgaaataggttggttgatctggatcttcttttgg | |
| | gtcattttgttcgctctgtatttcacaaattgccagaatctctgccaaccacagtggtaggtccaacttggtgttctga | |
| | atcacaggcttccccgggttgttctctaastaaccgaggcccggcacagaaatcgtaaaccgacacggtatcttttgtc | |
| | cgtccgccagtatctcatcaaggtcgtagtagcccatgatgagtatcaaaggggatttggttatgcgatgcaacgagag | |
| | attgtttatcccagatgctgatgtaaaaaccttaaccagcgtgacagtagaaataagacacgttaaaattacccgcgct | |
| | tccctaacaattggctctgcctttcggcaagtttctaactgccctcccctctcacatgcaccacgaacttaccgttcgc | |
| | tcctagcagaaccaccccaaagtttaatcaggaccgcattttagcctattgctgtagaaccccacaacataacctggtc | |
| | cagagccagccctttatatatggtaaatcccgtttgaacttcgaagtggaatcggaatttttacatcaaagaaactgat | |
| | actgaaacttttggcttcgacttggactttctcttaatc | |
| 6 | AAATaaatggcagaaggatcagcctggacgaagcaaccagttccaactgctaagtaaagaagatgctagacgaaggaga | pFDH1 |
| | cttcagaggtgaaaagtttgcaagaagagagctgcgggaaataaattttcaatttaaggacttgagtgcgtccatattc | |
| | gtgtacgtgtccaactgttttccattacctaagaaaaacataaagattaaaaagataaacccaatcgggaaactttagc | |
| | gtgccgtttcggattccgaaaaacttttggagcgccagatgactatggaaagaggagtgtaccaaaatggcaagtcggg | |
| | ggctactcaccggatagccaatacattctctaggaaccagggatgaatccaggtttttgtigtcacggtaggtcaagca | |
| | ttcacttcttaggaatatctcgttgaaagctacttgaaatcccattgggtgcggaaccagcttctaattaaatagttcg | |
| | atgatgttctctaagtgggactctacggctcaaacttctacacagcatcatcttagtagtcccttcccaaaacaccatt | |
| | ctaggtttcggaacgtaacgaaacaatgttcctctcttcacattcggccgttactctagccttccgaagaaccaatasa | |
| | agggaccggctgaaacgggtgtggaaactcctgtccagtttatggcaaaggctacagaaatcccaatcttgtcgggatg | |
| | ttgctcctcccaaacgccatattgtactgcagttggtgcgcattttagggaaaatttaccccagatgtcctgattttcg | |
| | agggctacccccaactccctgtgcttatacttagtctaattctattcagtgtgctgacctacacgtaatgatgtcgtaa | |
| | cccagttaaatggccgaaaaactatttaagtaagtttatttctcctccagatgagactctccttcttttctccgctagt | |
| | tatcaaactataaacctattttacctcaaatacctccaacatcacccacttaaacaG | |
| 7 | cagccattaatctcacctcagtttttgaatcagtagaattttTaatgaaacaaacggttggtatattatttgatagAgt | pFLD1 |
| | TgccaaatttccaaaGatAaaTttttcatcaggtaatatcCtgaataccgtaaCAtagtgactattggaagaCactgct | |
| | atcaTattatatttcggataAaaatccaaaccccagacCgaCctcttgagtctcaactcCaagtcagccgcAactTtaa | |
| | ttatcCgtggattGggagCtagtTtggacaaCgcatcagtataAtataactttacggttccattatcagacgctattgc | |
| | aagaacttcctttccattgatctcGccaatGcgGcagtaattgatatcGtaGggtaggtctggaaaGacGctggcgctt | |
| | gtGtcccattctgcaggaatCtctggCacggtgCtaatggtagttatccaacggagCtgAggtagtCgAtatatctgga | |
| | tatgccgcctataggataaaaacaggagagGgtgaaccttgcttaTggctactagattgttcttgtactcTgaattCtc | |
| | AttatggGaaActaAactaatctcatctgtgtgttgcagtactattgaAtcgttgtagtatctaccTggagggcattcc | |
| | atgaaTtagtgagaTaaCAgagttggGTAACTAGAGAGAATaatagacGtatgcaTgaTtActacacaacggatgtcgc | |
| | actctttCcttagttAaAaCtatcatccaAtcaCaagaTGcgggctGgaaAgacttgctcccgaaggataatcTTctgc | |
| | tTctatctcccttcctcatatGgtTtCgcagggctcatgccccttCTtccttcgaactgcccgatgaggaagtcCttag | |
| | cctatcaaAgaattcgggaccatcatcGatttttagagccttacctgatcgcaatcaggatttcactactcatataaat | |
| | acatcGctcaaaGctccaactttgcttgttcatacaattcttgatattcacaGg | |
| 8 | CTTCAGTAATGTCTTGTTTCTTTTGTTGCAGTGGTGAGCCATTTTGACTTCGTGAAAGTTTCTTTAGAATAGTTGTTTC | pILVS/ |
| | CAGAGGCCAAACATTCCACCCGTAGTAAAGTGCAAGCGTAGGAAGACCAAGACTGGCATAAATCAGGTATAAGTGTCGA | pEM72 |
| | GCACTGGCAGGTGATCTTCTGAAAGTTTCTACTAGCAGATAAGATCCAGTAGTCATGCATATGGCAACAATGTACCGTG | |
| | TGGATCTAAGAACGCGTCCTACTAACCTTCGCATTCGTTGGTCCAGTTTGTTGTTATCGATCAACGTGACAAGGTTGTC | |
| | GATTCCGCGTAAGCATGCATACCCAAGGACGCCTGTTGCAATTCCAAGTGAGCCAGTTCCAACAATCTTTGTAATATTA | |
| | GAGCACTTCATTGTGTTGCGCTTGAAAGTAAAATGCGAACAAATTAAGAGATAATCTCGAAACCGCGACTTCAAACGCC | |
| | AATATGATGTGCGGCACACAATAAGCGTTCATATCCGCTGGGTGACTTTCTCGCTTTAAAAAATTATCCGAAAAAATTT | |
| | TCTAGAGTGTTGTTACTTTATACTTCCGGCTCGTATAATACGACAAGGTGTAAGGAGGACTAAACC | |
| 9 | Gtgaatttgtcacggaattgaccaagaggtcagacgatcctgtatcccattgagccgttatgctttgtgggggaaaccc | FGH1 |
| | tatttctatcgtactaagaaaaccaatggtgaactcatattcggtatcaatggcgacgattccagcatagcctgtagac | |
| | agtaacaacactagggcaacagcaactaacatatcttcattgatgaaacgttgtgatcggtgtgacttttatagtaasa | |
| | gctacaactgtttgaaataccaagatatcattgtgaatggctcaaaagggtaatacatctgaaaaacctgaagtgtgga | |
| | aaattccgatggagccaactcatgataacgcagaagtcccattttgccatcttctcttggtatgaaacggtagaaaatg | |
| | atccgagtatgccaattgatactcttgattcatgccctatagtttgcgtagggtttaattgatctcctggtctatcgat | |
| | ctgggacgcaatgtagaccccattagtggaaacactgaaaggcatccaacactctaggcggacccgctcacagtcattt | |
| | caggacaatcaccacaggaatcaactacttctcccagtcttccttgcgtgaagcttcaagcctacaacataacacttct | |
| | tacttaatctttgattctcgaattgtttacccaatcttgacaacttagcctaagcaatactctggggttatatatagca | |
| | attgctcttcctcgctgtagcgttcattccatctttcta | |
| 10 | ggcagaaggatcagcctggacgaagcaaccagttccaactgctaagtaaagaagatgctagacgaaggagacttcagag | FDH1 |
| | gtgaaaagtttgcaagaagagagctgcgggaaataaattttcaatttaaggacttgagtgcgtccatattcgtgtacgt | |
| | gtccaactgttttccattacctaagaaaaacataaagattaaaaagataaacccaatcgggaaactttagcgtgccgtt | |
| | tcggattccgaaaaacttttggagcgccagatgactatggaaagaggagtgtaccaaaatggcaagtcgggggctactc | |
| | accggatagccaatacattctctaggaaccagggatgaatccaggtttttgttgtcacggtaggtcaagcattcacttc | |
| | ttaggaatatctcgttgaaagctacttgaaatcccattgggtgcggaaccagcttctaattaaatagttcgatgatgtt | |
| | ctctaagtgggactctacggctcaaacttctacacagcatcatcttagtagtcccttcccaaaacaccattctaggttt | |
| | cggaacgtaacgaaacaatgttcctctcttcacattgggccgttactctagccttccgaagaaccaataaaagggaccg | |
| | gctgaaacgggtgtggaaactcctgtccagtttatggcaaaggctacagaaatcccaatcttgtcgggatgttgctcct | |
| | cccaaacgccatattgtattgcagttggtgcgcattttagggaaaatttaccccagatgtcctgattttcgagggctac | |
| | ccccaactccctgtgcttatacttagtctaattctattcagtgtgctgacctacacgtaatgatgtcgtaacccagtta | |
| | aatggccgaaaaactatttaagtaagtttatttctcctccagatgagactctccttcttttctccgctagttatcaaac | |
| | tataaacctattttacctcaaatacctccaacatcacccacttaaaca | |
| 11 | gtcgaggasagggtcgtttcggggagttaaatatttttggctatgtagcagacatgtttcgacgctggcgtcgcgtcga | Hansunela |
| | tcggasaatattaccccaggaacaagcacttgcttgggttagccaccaccctgcgcaagcctttttgccggctctacac | poly- |
| | agggccaatgaaatctgggcggaatctgaaaccgatgaaacggacgacactggcaacaagctcactgcactattttttt | morpha |
| | tttctagtgaaatagcctatcctcgtctcgctcccctcatacctgtaaaggggtgcaatttagcctcgttccagccatt | Tk13 |
| | cacgggccactcaacaacacgtcggctaccatggggtgcttgggcaccaaaaggcctataaataggcccccatccgtct | (transke |
| | gctacacagtcatctctgtcttttttccc | tolase) |
| 12 | Aagatcgggttatccatgaaatcctttcaaggagtagagacctattcctcacgaaattcgcacatagctgcaagagttc | TLR1 |
| | gtggtgggtgcaacctcccaagaattacaaggctgaagatgggactgagatagaatgaacagcctcaaaaaagaaaata | (the late |
| | aaaccgtgagcctgagaacgaaagataagggtctagatgcgctcagacgtgaaacggcaacatcaattagttaaaatga | response; |
| | ccgtaaaggcaattgatgtacgccgctgaagcaactgaatgaatagttccaaagaagtaggctcgtcaatttaaaatat | promoter |
| | ctcgtagtcagttagtttactgatattcaggtgcttgacgcgcggtttgcacccatatcaaccaatcacacctcttgat | is S′ |
| | cggatcaaaaagactccagaaatccagaaaatcaaatgtgtggagctggaatcctaaactccttcagttcatcctcttc | to an |
| | cagctctgcctgcctcaacttagatcagggctgataaataattttcataccactaataaagtcacgtgaaattggtatt | unanno- |
| | aataacccctcctctgtcatagtgcattgcttatcgataatagggggttcgcgcaaaagaacagggctgtggccgcggg | tated |
| | tcccttccgaccccctttagttgtagagagcagcaaagcactagttgacagatttaaagcttcgccgcttcacgtggcc | ORF in |
| | ttcgaggttgccacatctgcaaaagttcacctatcaaggtttcagatcggttatcagtaacttagttggttcccctcca | the K. |
| | tgaatcgaaatcaaaacacactcctgaagcgcaaccttctgattggggattcaagcgagggtcacgatttttttttgaa | phaffil |
| | attagcttgaaaaagagtcaaagaggggtgtgactgggccaattttttcggatgccaccccctcttttctccccatgac | GS115 |
| | atctaaagaccgattgaaaaaaaaaaaaatcgccagggtataaattgtgtgaaattccctgatttaagtccaattactc | genome) |
| | ttcatatcagtctcattaata | |
| 13 | Ccggaggttggagattttgtatatataaaactttcttggagcttattaataaatgcgggatgcagtaaacttgcatata | GND2 |
| | tctattgtaacacttttgcaatagctgcatgccttgactcatcattcagtatcgtgtgaaaaccaatgatacatccgta | (late- |
| | cattcaaactacaaccttcctcattagtaattctttttgaatttttcggaacccgaagctccgcctatccccccaacta | firing; |
| | acacatcttccaatttggggggagaacacctagcaacatcacgatcattgcgcgaacgttcgcactgtatttttttctc | promoter |
| | ccaaacacccaacttctaggccaaatatccacttctcggggttctattcacccatttaattgttggccttasaagtcaa | for 6- |
| | ttgagttccaatcatagtccctagttgattgcttgtagcaaatgccacaacagtaggcatttacgtcctcacagtctct | phospho- |
| | tcccttgtccctcattgatacctctttattctcccccaccaccatacactaccttcctcgcaccccigtcatcacaacc | gluconate |
| | gcaatataatcgatgcgcggtttcttgcctaatccatcgtccaacagagaggtcgctctccttatatatatagttgatc | dehydro- |
| | cccctttttttctacccttgcaattttttttttgggaccaaagaaaagaaacaagactgatacasat | genase) |
| 14 | gtacgttaagtatgccaaaaacatcacaatagacaatacgccctacaacagaacgtcaagttgtacgcgagcaccgtta | RPL40B |
| | aatcacacgtaacatccaacacctttctttggtagggcatagccccaggtggcaccacgtgcataaaccattttacacc | (late- |
| | ccaacacccaccaatcctcgccctctggcatttgctcaaattttttaaccagcttctgattacataggtaaccagttca | firing |
| | attcataggtaagtcaacgccgascaacaagggaaatcacccatccgctcacatcagtccagttgaagactaacattaa | promoter |
| | aagacaaaa | for ubi- |
| | | quitin) |
| 15 | gtcaaaacagtagtgataaaaggctatgaaggaggttgtctaggggctcgcggaggaaagtgattcaaacagacctgcc | PMP20 |
| | aaaaagagaaaaaagagggaatccctgttctttccaatggaaatgacgtaactttaacttgaaaaataccccaaccaga | |
| | agggttcaaactcaacaaggattgcgtaattcctacaagtagcttagagctgggggagagacaactgaaggcagcttaa | |
| | cgataacgcggggggattggtgcacgactcgaaaggaggtatcttagtcttgtaacctcttttttccagaggctattca | |
| | agattcataggcgatatcgatgtggagaagggtgaacsatataasaggctggagagatgtcaatgaagcagctggatag | |
| | atttcaaattttctagatttcagagtaatcgcacaaaacgaaggaatcccaccaagacaaaaaaaaaaattctaag | |
| 16 | ATTTAAATgtaAGATCTttatataaatgaatctacatggtgtgttttatttagatcctccaaaccaaggaaagaaacta | PMP47 |
| | aacttatctccggacttacgagtcaaataactatccgcagttccttggaactcagactttcttccataagcggtcatat | |
| | catctttggactgtgggaatcctggacgaatctttgaaatgtcataatcttgctctctatctccaagcacagcgtccgg | |
| | tasatgctggttcttctttctcagatgaatcttggatttaacaaataaagccgtgcctatggctaatgtactcaaaaac | |
| | aaagtctgcttccagaatttcgcaaacgatggaatgccatttcctgtaaatgtactcattgaacctatgtttgattaaa | |
| | gttggtgtgaagtcatcaaacgagagtaaaatcagatactcgtgcaccggccaaaattgactgagctaatctctgctgg | |
| | cttgacatccgaacacaacasataggcgacaaatcttaactatctaatcgtaggctatggtagaactttgtgggggtag | |
| | aggaagactacaacagcaagacaaaacaaaagagtcatagtttgactctctgcttttttcttctttctcttctttttct | |
| | tcctccatattcgttatttatttcgaactggaCAACTAATTATTGAAA | |
| 17 | TCCAGTGTAGCACTAAAATCTAATATCTTCGGCTTTATACTTTTTTGTTCATCCGAAAGCTTACGAACAATTCTTTCTC | P1 |
| | CTGTTTTATTGTGGATATAGACAATTTCGTCAGTTTCTTGGAGAGAAGAGTTATTTCCGGTTTTGGCTGGCCCTATAAA | |
| | CGGGTTCTTGGATTTGGATCTAGTAATAAAAATGTCACTGTCATTCTCGGAGCTGAACTTTGTGTTGTACGAAGATGGG | |
| | TTGTTCCACTGTTTTGCCAGCTCTTCATTGATGATTTTCTTAGTGGGTGTTCTTGGAGGTTCACGTTGCCTATAATCTT | |
| | GACGTTCTTCTTCATCACTATCGATGCCATCAAAATTAAGCGTCCTTATTGCAGGCTTTTGTGATTTCAACTGCAATCC | |
| | TTCTATCTCTTCATCAGAGCTTTCGAACTGAATACTATCACTCAAAACTGGCGACATTGCACATTTCCGCAAACCATTT | |
| | CGGGAATCTATGCTAGCTCTTCTAGACGATAAAGAACGACCGGAACCAATACGGGGTTGTGCAGGTGGGAATAAATATG | |
| | TTGGTTTGGATTCTTGACGTGAAGAAGGTATTCTAGTCGATGAAGTGGTTGATAAGGATATGGCGTCACTGAGTTGTTT | |
| | TCTTTTCCTATGTTGCGGTGTTGGGTCAGGAGTTAATTGATTCACCTCCATAACTCTGGAATTTCTTGAATGTGGGGTT | |
| | TTCAGATGGGCATCTTTCTTGACGGGGTTGTGAGTAACGGAGGAACCTGGTGTCTTGGGTGTGAACGGTGTTTGAGCCT | |
| | GTACGCGGTTACTTCTGGGGGGAGTACTCGGAGTCATGAGAGCCATTGATTAGAAGGTGAATGAGGGAGTCACCACTCT | |
| | AAGCAAACAAAATGAGGTCGAAGCAAAAAATAAAGTAAAGTAGCACTTCTGGCAGGTTAGATCAAAGAGTGACGGGAGA | |
| | TTTGAAGATGGCTGGTTTTTCCTTAGTCTTGGAAGAGGTTTGTGTGGGTATCAGCGAATATTCCCCGATTAGGCAAATT | |
| | AGTTGCATTGAAATTAACACGACATGGTGATTTGTGGTAACAAATATCTATTGGTGGTTGGTGTGTGGGTGTAATAGTG | |
| | GTCGTGTCATGATGATGGTGTTCAGGTGTTGTCATAGATCGGTCTTCAGTAAGAGAAGGAAGCTTGGTGACGATCACAG | |
| | CTATGATGTAATAGAAATTGCTAAGCAATTGTGAGGTGTGATGTATTTTGCAGAGCAATTGTGCGGTACAACGGGGTGT | |
| | TATTGTCTTCACAAGGCATTTATTGCGAATTTCGTAGTTGAAAGAATATTTTAGCACAGGGTGCTTGACCCCTATTGTT | |
| | GCTCGCTAAACCATGATTGCTAAATGATGACATAGCAATCACTTTACTAAGATTGCTATAAGGACACCTTTCTTAGTAT | |
| | AAATGGACACTCTTTTCCCCTGCTAAACTTCTTTTATTTTTCACACTTAAACAGTTACAAAACACAAACACAACTAGAA | |
| 18 | GTGCTAAAATCTGAGGTTTACAAGCTGTGATGTTCCCCTAAGATCTCACAATCGAACAATCGOGAAGCCAATGCAAGTT | P2 |
| | GTTTAAGGGGAAACGACTCACTATTCCTGAAATTAGTATTCAAAACTTGGTCCGGAAGAACAATGAGGCGGCCGTTAAA | |
| | ATACTCACGTAAACGGTGTCTACAAGCGCATTAAAATCCGTTTGAATTCAAGCAAAAGCCACCAGAGGCTTATGCTTGG | |
| | TTATACCCAGCATTGACCTTTGGTATGAGCATCTGAAAAACAACCAGGTGTTGCAAAGTTAAACATCCTTCTTTGTTCA | |
| | TATAGAACCCACTATTCATGGTACTCCCCAATCGAATTTCACATTCTGGTTTTGAAATTACACACCACGTTAGCTTATA | |
| | AGATTTCATATAACTTATTGATATACGGTTTCCATTGTTCGAATAGTTGAGGTTGTATGTAATTCGATTGAAGGGGCCA | |
| | TTTTTGTTTCCTACTTTTCCTGGGAGCTTATCCGATGCGCTTCAAAGCTGGAATTGTAAATATAGAGAAAAAGAAGGAT | |
| | GTTGTTTTATTCTTGAAAGAGTATAATTTTACTTCTAGCAACTCTCCCACTTCGCTTGACTTCATTTATTTCTTGGGCA | |
| | CATAGGCGTAGTAATCTAGACCAACAGATAATTTGCCGGAATGATATAGCGATTGGAAAATGAACTGAAATTTTTTGCT | |
| | GTCITTCAATTTGACGGGCAGTTCATCAGTGACCGACCATATAAATACGTTGAGAATGTTATTCTTCCTCGTAGTTGAA | |
| | GTGGCTTCATAATTTCAGAACTCAATAGATAAACTAGGATGTTTTAAAGCAATTAATGCTCACAAGTAAGGAGCGACTC | |
| | TCTTGCTTTTCGAATACTAAAAGTATCGTCCCAACCCAGAAAAAAAGACCTCTTAACTGCAAAATAAACTCTATATATT | |
| | TCTTCTAAAACAGTTTCAGGTTGGATAGTATCGCATTCTCATCACTTCTAACTAGTAGGCCATGAGATATATTAACGTT | |
| | TACTTGAGTTCTAAGTTCTCCGAATTAGATGCACAGCACAAACAAGATTAGGTTTCACTTGGTACAAAATACGAACAGA | |
| | GTTTAAGGTCGTAATTTCATTTCGTTATTGATCCCCACAATCTATTCTTATCACAGTCATCAGATAGTOGCGAAAAAGC | |
| | ATGCAGAAAAGGGGGTCGTCCCTATCTAAGTTGTAGCATTACAACAAATATGACTACACTCAGTGTCGCAATCGGTATA | |
| | GCCAACGCTGCAAAATGGATTCTACTGAGAATGGTATGATGATCCCAGGATCAATTTCCCAAAAATTAAAAAAAGTAAA | |
| | ATAAAAAGCATCAGATATTAGGGAGGTGGTAAGATTGCTCTGCAAGCGATCACGAGATTTTAGGTTTTCCTTTATGTAC | |
| | TATATAAAGCGCAGATTGGATGCCGCTTTTCCCTCCTGGGCTATGATAATATAGCGAACGAAATACACGCCAAAATAAA | |
| 19 | TCACATTCATAGCATCTCTCGCCTGCAATAGCTTCCACGATAGGAATATCTGTGAAAGTGAACATGCTATTTCGATGAT | P3 |
| | ATAAGACTTTAAGATCTGGCATGTTTGTGTTGGAGGTTACCCTGGGGTCAATAACCCTAATTATCTCCTTCACTAAAAA | |
| | TGATGAAGATTCTTCGGATTOGTTTTTGAACAGAGTTAATGCCATTTCTTCGTCAATAGAAAAATCAATATCTGGTATC | |
| | TCATCTTTTACATATTGAGGATTTAGTTTTCTTCCCTTTGGATAGTACATTATGATCAATGTATTCCTGTCTTTATTGA | |
| | TAAAGTATTGGCATTCTGCTTCTTGTACACCTTTGAATTGTTTGTCTGGAAGTGACTGACATTTTTCCACATTGCTAAC | |
| | GGTTTGGCACGAATTACATCTAAATAAAATGTCTTCTCCGGATTCGTGTATTAAGTGATACTCCAATGATAAATCCCCA | |
| | CCTATCGAACCAGAATCGGCATTGGCCACAGTCACAGGTAACTTTAGGTCTTGAAAAATCCTTCTATAGGCTTCATTGA | |
| | CATTGTCATAAGACTTAAGACCATCTTCTTTGGTCAAGTCAAAAGAATAGGCATCTTTCATGAGAAACTCTCGTCCTCT | |
| | CAACAAACCTCCCCTAGGTCTCAACTCATCTCTATATTTGCGGGAAATTTGGTACACGAGAAGGGGTAAATCTTTATAT | |
| | GACGAACATAAGTCACCAACTAAGTTTGTGATTTCCTCTTCACAAGTTGGCACTAAACAGTAGTCTCTATCCTTGGAGT | |
| | CTTTGAACTTGAACAATTCATTGTTGTCCCATCTCTTAGTTCTCTCCCATAAATGCTTGGAAGACAGGCTACTTAATTC | |
| | CATTTCCAGCCCACCAGCCTGATCCATTCTTTTCCTAATTACATTTTGAAGCTTTTTATAGGTACGGAGTCCTAATGGA | |
| | AGCCAGTGAACTATTCCTGCTGCAGGCTGGTAAATAAACCTTGATTGAAGGAGCATATCATGAGTAGTAAGGTCCTTTA | |
| | CAGAAAATAGTTTACTTCCTTGAAGAGAAGTAGAATAAAACCTCATGTTGGGTCTCCATGAAAGGTTCAAAGGCATTGA | |
| | TCCTTTAGGTACTTCAGGATGTTTAAGTCATCAAACTGTCCATCAAAGGTAGTATAGTATTTACCATCTAGATAGTGAT | |
| | GTATGGGTGTAACACAACATTTAAATGTTGTAAATTAACATTAGGACTGAGTCCGGAGATGCTATTGTCACCTAAATCT | |
| | ATTAGAAAGCACTTCAGTTATATCATCGATAGAGGTTTGAAGATAAACCTATTGTTGATAAATAACCCCATTACCCGTT | |
| | TACGTAGCAAGGTTCAAAAATTTGCTTAGATCGGAGCTAAAAATTCGACTGACTTCTTTCGAAAATGTGGATTATGCAA | |
| | GCAACGTTGCTATCGGAATAGTATATAAGGTCGATCTGCCCCATTACAAATTGTAAAGCAACAAACATCCTACGCAAA | |
| 20 | TCAGTTTCACGGTTATGTGAGCTGTCTCCGCGTGAGGCAGTAACCTCTGTGTCATGGATACAGGCTGGTACACATTTGG | P4 |
| | CAGTAGGAACACAATCTGGTTTAGTTGAAATATGGGACGCCACGACGTCCAAATGTACAAGATCAATGACTGGGCATTC | |
| | GGCCCGAACCTCAGCGCTGAGTTGGAACCGTCATGTTTTGAGTTCTGGTTCAAGAGATCGCAGTATCTTACATCGGGAT | |
| | GTACGTGCAGCAGCTCACTATACAAGTCGCATTGTTGAACACCGCCAAGAGGTTTGTGGCTTACGTTGGAACGTGGATG | |
| | AAAACAAGCTGGCCAGTGGTTCCAATGATAACCGTATGATGGTATGGGATGCACTGCGTGTAGAACAGCCCCTTATGAA | |
| | AGTTGAAGAGCATACTGCGGCTGTTAAGGCGTTGGCATGGTCACCTCATCAACGTGGAATACTGGCTTCGGGTGGAGGT | |
| | ACTGCTGACAGACGTATCAAGGTGTGGAATACTTTAACAGGATCCAAGCTGCACGATGTTGATACTGGATCTCAAGTTT | |
| | GTAATCTCTTGTGGTCTCGCAATTCTAATGAATTGGTAAGTACTCATGGATATTCTCGAAACCAAGTCGTTATTTGGAA | |
| | ATATCCGCAAATGAAGCAACTAGCATCTTTGACTGGTCATACTTATCGAGTCCTITACCTTTCCATGTCACCTGATGGA | |
| | ACTACAGTOGTAACGGGGGCTGGAGACGAAACTTTAAGATTTTGGAACTGTTTCGAGAAGTCACGACAAAGCGGAGGAG | |
| | GATCAATATTACTAGACGCTTTTAGTCAGCTTCGTTAAATTACCACCAAATTTGGTGCAAAAGGGCCCATATGGTGCTA | |
| | CAACCAAAGGAACTTTCTAATTTTGATAATGATGTCATTTCTCTCATCGGGATGAAAATAGAAGTCGAAAGGATTTTTG | |
| | TCACTATTTCAAGCCCCACCTGCAGCTGGCAGCATTTCTATTGTTTATGCATTGTCATTTATGGGAAAACTAAGAAAGT | |
| | TCCTCTCCACCCGGACTCCACTGGTAAATATGCGATATCGGAATCATGACCAACCCATATTTTGATCCTAATCATTTCG | |
| | GTTCTAGTCTCCGATCGGACTCCGTAAAACTGCGGAGTGAACTCCAACGGAGAATACTGCAGCCAATCTCATATTTCAT | |
| | TTGTTATTTGTCCCTCAACTGTCTCGATAAGGTCATCTGTGTTTGACTAGATGTTCGTCATTGGCATGTCAAACAAGGC | |
| | TAGACCTTACAATCATCTCTTACGAATGTAAGTGAATGTAACTATATTTTCCTTGCTACTTTAACGAGGTTAACCAACC | |
| | CCCGCACATCCCCACACCACCGCTCTTGATAAGCATCTCCGAAAATGCATGACGCGACAACTTCAAGCATGTTGTATTT | |
| | ACTGAGTTTTCAGCCTCACTATCGATACCTCTATAAATAGAGGCACTTTCGTCTCTTCTCCCTCCCCACAAGAAACCA | |
| 21 | AGAAGTACTGTTATGAATCGATCGACGTGACATGTTGTTGATGGTTCTGACTTCTTGATGTCCGCGTTTTCTGTCTCTC | P5 |
| | AATAGTGGTGTTCGGGGGAAGTATGGTTCTAATACTTAACAGGTAAGATGGTTGCAATGAGCACCTGGTAAAGCAACTT | |
| | GAATTTCCTGCCCTGTCTCCGTTAAGTTATATTCGACTCAAGGTCCTTGCTTCCTGTCTGTTCTGTAAAACTTCCCTTT | |
| | GGTGTCTTCTATATCAACTTTAAAAACAAGGTAGTGTGTCGAGCGATAGTACTGTGTCTTTTTCCCTATGAAAAAAATC | |
| | GCACCATCCAAGACTTCTCACCTTCAACAGCTTCAACATCATGTTCGGTCCTTTTAGAGCTACGCTGGTCGATCTAGGA | |
| | GGTCTGCTATGGAAACGTCCTTGGAGAATGTCCAAACCACAGAAATATAGACTCCGCAAAAGAATGCAACTTGTAGACT | |
| | CCAATATCGACATTATTTACCAGGGACTGACTGAGGAGGGTCTGTCTTGCAAAGTGATAGATAACTTGAAACAAAACTT | |
| | CCCAAAGGAGCATGAAGTGCTCCCCAAAAACAAGTATACCGTGTTTAACAAGACAGCCAAAAACTATAGAAAGGGTGTT | |
| | CATTTGGTTCCAAAATGGACCAAGAAGTCTTTGAGAGAGAACCCCGAGTTCTTCTAATTGCACATTTCTTCCTGTTCAT | |
| | AGATTATCCCACACATAGTTGCTCACAAAAAAATCACTATAATTTTCCTCCACCGGCAGTATATCACTAACACCTTTAT | |
| | CTTTATTGTAGATTATAATCTGATCTTTATCCTTAGATGTATCTATCATCAACCCCATGCTCTTGAAAAGCTTGAGTCT | |
| | TAACACTGTCGAATCGTAGTTTTCTTGTAGATCATTCGATATCACTGCTTTTTCTTGCTCTTCTAATTOGTTGAGATTC | |
| | TGGGTCAAACTAGAGATTGAATTCTGAAGGTGATTCATGTTCATCTCCAGATCTGTTATTGATTTTGCTAATTTAAATT | |
| | TTTCGTGTTCAAGCTCTTCGATACTCTTTAGGGTCTGTTGACGGTCTTCTGTTTCCAATAATTGCTTGTTGAACTCTTT | |
| | AAGTTCGTCTCTCTGTTTACTGATACGTGACAACAAATCTAGCTGGTGATCGAGTTTAAGTTTCCGTTTGGAGCTCAAC | |
| | AGAGAAAGATTTTCATTAATTTGGTTGATAGTTTGCACGTCCGGTTCGATCTGAAAATTCTCTATAGTCGACCTGATTA | |
| | AGGACACAGTCTCTTGAAGATCGGACATTGGATTTATGGAGAAGGGAGATCAAAGCGGAACCAGTTGCACTGTTTACCT | |
| | TTCCAGTCGAGATACTTATCCCACAGGGCCCTCACTTTCCAGGCAGAAGTCACCTAGGAGGCGCATCCCTCCGTTTGCT | |
| | TCCCTCGCGACAAACTCCCCTGTAAAAGAAAACTTCACTGAATCGTAC | |
| 22 | GTCCTTTCCAAATTTTTGGTTGAAGGCATCGCTTAAATTATGAGCAGGATCGGTGGAAATAAGCAGGTATTTCTTGTTA | P6 |
| | GGATTGTGAAGGGCAAGCTGGATAGATATAGAAGAAGATGTCGTGGTTTTACCGACACCCCCCTTACCTCCAACAAAGA | |
| | TCCACTTCAGCGATTCGTGGTTCACAATTGATCGCAAACTTGGCTCTGCCTCAATATCCATGGTTGATGTCTAGTTGAG | |
| | TGGCGTTTGTGGTCTCTTGATGAGTTCAAGGCGAAAGAATATGATAGGAAAGCATGGTTTGAACTTTTCGCGAAAGAAG | |
| | GAATACTGTTCCGCGAGAAACTCCCCGGTGCCAGAACCTTCCATTGAGGTTAATCGGTGGGAGGTGTTCGAATGACAAT | |
| | GTCAGACAAGGCGAACACGTCTTGTGACACCAGCTGGACTAAGAAGATTCGGTATGCACCGAAGAAGAAGGCCGTGTCT | |
| | CAATTGGCAACTTTGCAACAAACTACGGAGGAAAAGTCTCACAAGCTTTTAACCAAGTTGAATCACGACGACAACGATA | |
| | AAGAAATCCTCAACCATCTAACACATGAAGTACAAAGTAGAAATGTGATCTTATTGGACAAACTAGAGGAGCTCAACAA | |
| | GGAACTGGGCTGGATTAAAGACCGAAAATGAGGAACCATGAGCACTGGGCGTTTCCAGAAAAACTGCAACCAACGATGG | |
| | GAAAATGATACCACACTACTATGGTCACCCCACATTGTGAAATTTCAAACCAAAAAAGATCAACCCCATAATTCCCCAG | |
| | AGGGTTTTCCCAACAATTTTCCAACGGACTTGATAATGAGTCAGATCATTTGAGCATATTCATCTTACCCCTTATTCCG | |
| | TGACAATTTACCTATTCCATTCAAAGCATACGGTATCCCGTGACCTTCTCATGGAGATCATTCTCCACCGATACAGCAT | |
| | ATACACAGATATACCCAACTAATATCAATTGGACCTTGATATGGTCGACCTTGATGGTCCCGTCCAACCTTAAAACTTA | |
| | GTTTAATGCTATACTTTCGCCTTGAACCAAATCTGTCTCCCCCTCAATCATCTCTATGCAAGAAGGTCAACACTGATTA | |
| | CGTGAGCAACAGCCAGCAATCGTTCGAGTCCCCGCCAAAAAAGGCGGAGTTACTGCTCCTTGTGACCACACCCCCTGAG | |
| | ACCACGTCCCTAAACGATCCTTGTCGGTTCCTTCGTCCAATTGGCAATTGCCACGCATACGTGAATCGTTATTGTTTCG | |
| | CCTACCTTGCGTCATTOGTTCCAGAATGTTCGACATACTCCTCTAGAACATACCGTCACACCACCATCTTAAGTTATCT | |
| | TCACGTGACCATGACGTACATTGTAGTTGACTACCCCATTCTCATCATTCCGATGCGGCCAAAAATCTCTATATAAAGA | |
| | CCGTATCCCCTAATATTCTCTTCTTGTTAAGACATTAACTTAGTTAATTCACCAATTACTCACTTATAAACAAACAAA | |
| 23 | GTTTCTCTTGGGGAGATACTTTTTTCGCGTGCTCCTCCGTGCGGAACTTCCTTCTGAGCTTCTACCTCTCAGATTAGTC | P7 |
| | TAATCGCATCAGGAATAAGACTGAGAATGCTTTTAAGGAGAGGCTTGAGATTGGCTAATTGOGTTCCGAAGTACTCTTT | |
| | CAAAAGGAGTTATACCCCTCTCAACTACGATTCTCTAAAGAATTATCGTAGGCATGCTCAGGCGCCTCAACCCCATCAG | |
| | TTTGACGCCACTAGATGGGACCAACAACCAGTTACTAATGAGCAAGGAGTAATACTCCCATCCGACTCAATTGCAAACA | |
| | TTCTGAGACAACCAACTCTGGTCATAGAACGGCAAATGGAAATGATGAATATATTTTTAGGATTTGAGCAGGCGAACCG | |
| | ATATGTTATCATGGATCCTACAGGAAGTATTTTGGGTTACATGCTAGAAAGGGATCTGGGCATCACCAAAGCTATATTG | |
| | AGACAGATCTACCGTTTGCATCGACCTTTTACAGTGGATGTAATGGATACTGCAGGAAATGTATTAATGACAATCAAGA | |
| | GGCCGTTTAGTTTCATCAATTCGCACATCAAAGCTATATTACCCCCTTTCAGGAACAGCGACCCAGACGAACATGTAAT | |
| | TGGAGAATCCGTTCAAAGCTGGCATCCTTGGAGACGAAGATACAATCTATTTACAGCACAAATTGGCGAAAAGGACACT | |
| | GTCTACGATCAGTTCGGGTACATTGACGCACCGTTTCTTTCCTTTGAGTTTCCTGTACTTTCAGAATCTAGGCAAACGC | |
| | TAGGTGCTGTCTCTAGAAACTTCGTGGGCTTTGCAAGAGAGCTTTTCACAGATACAGGAGTTTACATCATCCGTATGGG | |
| | GCCTGAATCTTTTGTAGGGCTAGAAGGGAACTACGGGAACAATGTGGCCCAACATGCCCTTACGCTGGACCAAAGGGCT | |
| | GTATTATTAGCCAATGCCGTTTCAATTGACTTTGATTACTTTTCTAGGCACTCGTCACACAGTGGTGGCTTCATTGGGT | |
| | TTGAGGAATAGACAGGGTCTCGTCAACTCAGCTCCTGCCACCAAACCAATCATTGATCAACGAGCACACTTTTGTCCAC | |
| | GTGAGATCGCTTTCGCTTGCAGAAAGAGCAATGCATGAAAACGGCAAACGCAAAACGAGCAAAAAAACGAGTAAATAAC | |
| | TACAATTTCACCACCAACAGGGTCAAAGAGCTTTTGAGACACTATAAAAGGGGCCCTTTCCCCCCAGGTTCCTTGAAAT | |
| | CCTCATTCAATTATGTTTTTTACTCATAATTTGACTCAATTGGCATCTTCTTCTTTGTTCATATACAGTAATTGATATG | |
| | ACGCTTAGTCATTATTAGTGTTCTCGACTAGCAGTGGCGAAAAAAGGGGGAGTTATTTTCTAGAACCGACCGCAAACTA | |
| | TAAAAGAAAGCTGCCCCTCATATACCTTTCGAATTCTTTATTTTCTGTGTTTCTTCCCTATTTAACATCTACACAAAA | |

TABLE 2
Terminator sequences

| SEQ ID | | |
|---|---|---|
| NO | SEQUENCE | ANNOTATION |
| 24 | TCAAGAGGATGTCAGAATGCCATTTGCCTGAGAGATGCAGGCTTCATTTTTGATACTTTTTTATTTGTA | tAOX1 |
| | ACCTATATAGTATAGGATTTTTTTTGTCATTTTGTTTCTTCTCGTACGAGCTTGCTCCTGATCAGCCTA | |
| | TCTCGCAGCAGATGAATATCTTGTGGTAGGGGTTTGGGAAAATCATTCGAGTTTGATGTTTTTCTTGGT | |
| | ATTTCCCACTCCTCTTCAGAGTACAGAAGATTAAGTGAAACCTTCGTTTGTGCG | |
| 25 | AATTGACACCTTACGATTATTTAGAGAGTATTTATTAGTTTTATTGTATGTATACGGATGTTTTATTAT | tAOD1 |
| | CTATTTATGCCCTTATATTCTGTAACTATCCAAAAGTCCTATCTTATCAAGCCAGCAATCTATGTCCGC | |
| | GAACGTCAACTAAAAATAAGCTTTTTATGCTCTTCTCTCTTTTTTTCCCTTCGGTATAATTATACCTTG | |
| | CATCCACAGATTCTCCTGCCAAATTTTGCATAATCCTTTACAACATGGCTATATGGGAGCACTTAGCGC | |
| | CCTCCAAAACCCATATTGCCTACGCATGTATAGGTGTTTTTTCCACAATATTTTCTCTGTGCTCTCTTT | |
| | TTATTAAAGAGAAGCTCTATATCGGAGAAGCTTCTGTGGCCGTTATATTCGGCCTTATCGTGGGACCAC | |
| | ATTGCCTGAATTGGTTTGCCCCGGAAGATTGGGGAAACTTGGATCTGATTACCTTAGCTGCA | |
| 26 | acgggaagtctttacagttttagttaggagcccttatatatgacagtaatgctagtacgttttgttttg | DAS1aTT |
| | tttaattaataacttagtttatgttagcctagtatagactccatcaattttttttgttattacgtaagc | |
| | cgcgatgataatatctgatgaaaaattcctatcagaaaataatttatcaaaagtttcatgcgatatgag | |
| | actaagtagaatagggactcccaaagtgtcagtcacaagggtcattcccgttcgtaatgtggtgatagc | |
| | gaggagaaaacctgtcagagcaagtaacaccgacgcaaagacatggctaatgaaagaagagcagagaag | |
| | aataagacagaaggagcaggagatgaaacaaaggctagaggaactagaaaggttcaaaaaaaagtacag | |
| | aaatcatatataaggaaagaggataggcatttggcacaagagatagaaaaggatcttgacataatcact | |
| | gatgattacaatttg | |
| 27 | Ggagacgtggaaggacataccgcttttgagaagcgtgtttgaaaatagttctttttctggtttatatcg | HpMoxTT |
| | tttatgaagtgatgagatgaaaagctgaaatagcgagtataggaaaatttaatgaaaattaaattaaat | |
| | attttcttaggctattagtcaccttcaaaatgccggccgcttctaagaacgttgtc | |
| 28 | Tcgatttgtatgtgaaatagctgaaattcgaasatttcattatggctgtatctactttagcgtattagg | Tdh3TT |
| | catttgagcattggcttgaacaatgcgggctgtagtgtgtcaccasagaaaccattcgggttcggatct | |
| | ggaagtcctcatcacgtgatgccgatctcgtgtattttattttcagataacacctgaagacttt | |

TABLE 3
Signal peptide sequences

| SEQ | | |
|---|---|---|
| ID | | |
| NO. | SEQUENCE | ANNOTATION |
| 29 | ATGAGATTCCCATCTATTTTCACCGCTGTCTTGTTCGCTGCCTCCTCTGCATTGGCTGCCCCTGTTAAC | alpha mating |
| | ACTACCACTGAAGACGAGACTGCTCAAATTCCAGCTGAAGCAGTTATCGGTTACTCTGACCTTGAGGGT | factor |
| | GATTTCGACGTCGCTGTTTTGCCTTTCTCTAACTCCACTAACAACGGTTTGTTGTTCATTAACACCACT | secretion |
| | ATCGCTTCCATTGCTGCTAAGGAAGAGGGTGTCTCTCTCGAGAAAAGAGAGGCCGAAGCT | signal |
| | | (seq 1) |
| 30 | ATGAGATTCCCTTCTATTTTCACTGCTGTTTTGTTCGCTGCTTCTTCTGCTTTGGCTGCTCCAGTTAAC | alpha mating |
| | ACTACTACCGAAGACGAAACTGCTCAAATTCCTGCTGAAGCTGTTATTGGTTACTCTGACTTGGAAGGT | factor |
| | GACTTCGACGTTGCTGTTTTGCCATTCTCTAACTCTACTAACAACGGTTTGTTGTTCATTAACACTACT | secretion |
| | ATTGCTTCTATTGCTGCTAAGGAAGAAGGTGTTTCTTTGGACAAGAGAGAAGCTGAAGCT | |

TABLE 4
Protein fused to secretion signal

| SEQ | | |
|---|---|---|
| ID | | ANNO- |
| NO. | SEQUENCE | TATION |
| 31 | GCTGAAGTAGACTGCTCAAGATTTCCAAATGCTACTGACAAGGAAGGAAAGGATGTCCTCGTATGTAACAAGGACCTTAGACCC | OVD |
| | ATTTGCGGTACGGATGGCGTGACATACACTAATGATTGTTTACTATGTGCCTATAGCATTGAGTTCGGTACAAACATCTCCAAA | (Seq 1) |
| | GAGCACGATGGAGAATGTAAAGAGACTGTCCCTATGAACTGTTCCTCTTACGCAAATACAACTTCAGAGGACGGTAAGGTGATG | |
| | GTCTTGTGTAACAGGGCTTTCAATCCAGTTTGTGGTACTGACGGTGTTACTTACGATAACGAATGTCTGTTGTGTGCTCATAAA | |
| | GTTGAGCAAGGAGCATCTGTTGATAAAAGACACGATGGTGGATGCCGTAAGGAATTGGCCGCAGTTTCGGTGGACTGCTCCGAA | |
| | TATCCAAAACCTGACTGTACCGCTGAGGATCGTCCTCTGTGCGGAAGTGACAACAAGACCTATGGTAATAAGTGTAATTTCTGT | |
| | AATGCTGTTGTTGAAAGCAATGGTACATTAACATTGTCTCATTTTGGTAAatgttaa | |
| 32 | GCAGAAGTTGACTGTTCTCGTTTCCCAAATGCTACTGACAAGGAAGGAAAAGACGTCTTGGTGTGTAACAAGGATTTGAGGCCA | OVD |
| | ATTTGTGGTACAGATGGTGTGACTTACACTAATGATTGTCTACTTTGCGCATATAGCATCGAGTTTGGAACCAATATCTCAAAA | (seq 2) |
| | GAGCACGACGGTGAATGTAAAGAGACTGTCCCAATGAACTGTTCTTCCTACGCTAATACAACCTCCGAGGATGGTAAAGTAATG | |
| | GTTTTGTGCAACAGAGCCTTTAATCCTGTTTGTGGCACGGATGGAGTCACTTATGATAATGAATGTCTCCTGTGCGCCCACAAG | |
| | GTAGAACAAGGTGCTAGCGTTGATAAGCGTCATGACGGTGGATGTAGAAAGGAATTAGCTGCTGTGTCTGTTGATTGTTCAGAA | |
| | TATCCCAAGCCTGACTGTACAGCTGAGGACAGACCTCTGTGCGGTTCCGACAACAAAACATACGGAAACAAATGCAACTTCTGT | |
| | AATGCAGTGGTTGAGTCGAATGGAACATTGACTTTAAGTCATTTCGGTAAATGT | |
| 33 | CTAGTAAAGGTGCCTCTAGTTAGAAAGAAGAGTCTGAGACAAAACCTAATTAAGAACGGAAAACTGAAGGATTTCTTAAAAACG | PGA |
| | CATAAACATAACCCCGCCTCCAAATACTTTCCTGAAGCAGCCGCITTAATAGGCGACGAACCTTTAGAAAATTACTTAGATACC | |
| | GAGTATTTCGGCACTATTGGTATTGGTACGCCCGCACAAGATTTCACGGTAATCTTCGACACCGGCAGTTCAAATTTATGGGTG | |
| | CCCTCCGTGTATTGTAGTAGTTTGGCTTGCTCCGACCATAATCAGTTCAACCCCGATGATTCCTCCACGTTCGAGGCCACGAGT | |
| | CAAGAATTGAGTATAACCTACGGCACCGGTTCCATGACAGGCATCCTAGGATACGATACAGTACAAGTCGGCGGCATTTCCGAC | |
| | ACCAATCAGATATTTGGCCTAAGTGAGACCGAGCCCGGATCTTTCTTGTACTACGCCCCTTTCGACGGAATCTTGGGTCTAGCT | |
| | TATCCTAGTATATCTGCATCCGGAGCTACACCCGTGTTTGACAACCTATGGGATCAGGGCCTTGTCTCCCAGGATCTATTCTCA | |
| | GTCTACCTGAGTAGTAATGATGATTCAGGCTCAGTAGTGTTGCTAGGCGGAATTGATTCTAGTTACTACACAGGTTCTCTGAAC | |
| | TGGGTTCCTGTCAGTGTAGAGGGCTATTGGCAGATCACACTGGATTCCATAACTATGGATGGAGAGACCATCGCCTGCTCCGGC | |
| | GGTTGTCAGGCAATAGTGGATACCGGAACCAGTCTGTTGACTGGCCCTACCTCTGCCATAGCTAATATACAAAGTGATATAGGA | |
| | GCATCTGAGAACTCTGACGGCGAGATGGTAATCTCTTGTTCTAGTATCGATTCATTACCTGACATAGTTTTTACCATAAATGGT | |
| | GTTCAATACCCCCTAAGTCCTTCCGCCTATATCTTGCAAGATGATGACTCATGTACAAGTGGCTTTGAAGGTATGGATGTACCC | |
| | ACGTCATCAGGTGAGCTTTGGATACTGGGCGATGTGTTTATCAGGCAATACTACACCGTGTTCGATAGGGCTAACAACAAGGTG | |
| | GGTCTAGCACCTGTTGCATAA | |

TABLE 5
Selection and other markers

| SEQ | | |
|---|---|---|
| ID | | |
| NO. | SEQUENCE | ANNOTATION |
| 34 | AGTGAAAACGAAAAGTGAAAATATCCTGAGGACGacttttattttttggctggtgctagcgctgcatgttcgttactagccgt | Ura3 |
| | tcaatacccattttctaaagttcagtcaatacatttagcaaggttggaagcgttggatattttcaacgagaactcctgggata | cassette |
| | agaaaagtaaattccgtctatattaccgatcatacttagatacattccaccagttggtgagcttacatgagaagtctaaacta | (promoter, |
| | tcttggacccgctggttttataaagggttcgttaggaatgcgttaactaccattccagcaacatccgtggggcttctggtgtt | gene, |
| | tgasatactgcgtcasaaattgagcgatgaaattgaagatcgattcagttgaatcgcccgaaacaattgatcccctgtacata | terminator) |
| | cttgtaatttacctcagaatattttggtaagcttcccacccagcttttctataccgttcaccttcttttaagggatctctgcc | |
| | cttgccaaacaaaccacgacctacgattatgatgtcagtgccagtggaaaatacttgactcactgttcgatattgttggccta | |
| | gagcatcaccagtgtcatccaaaccaacacctggtgtcataataatccaatcgaacccttcatcttgtcctcccatagaattt | |
| | tgagcaataaacccaatgacgaattccttgtctgattttgcaatttctacagtttcttcggtgtacttaCCATGGgcaattga | |
| | tccctttgacgacagttcagccaacatcaatagtccccttggttgatctgttgtctcagtggctgcctcctttagacccttta | |
| | caattccactaccaatgacaccatgagcatttgtaatatctgcccattgtgcaatcttgtagacacctccttgatattgatgc | |
| | ttgacagtgttgcctatatcagcaaactttctgtcctcaaaaattaaaaacttgtgtttctttgatagttccaataaaggcag | |
| | aatagttccatcatacgtgaagtcatcaattatgtcgatatgagtcttggccaaacagataaatgggcccaatttatctagaa | |
| | gctccaataattctttagttgttctcacgtcgactgatgcgcataggttactctgtttctgttccataagcgcaaacagtcgt | |
| | cgtgccacaggtgattgatgagtatttgctctctcggcataactgcgagccattgtctaggtatctatccctttgatcaggtt | |
| | gatgttaactcattagagtggatcaatgcgaaggataggtgcgacgtgtaccgtccaaaaaacttttttcttcaatcttgaca | |
| | aaaactggtaacagagagagcaagtgctaactctaccccaaccaagtacatcacaaaatggacgcattgaacgctaaagaaca | |
| | acaagagttccagaaactcgttgaacaaaaacaaatgaaagacttcatgcgtctttactccgatttggttagcaaatgtttta | |
| | cagactgtgtcaatgattttacatctaacaagttgacttctaaggaggaaggctgcatcaacaagtgtgcagaaaagttcctc | |
| | aagcacagtgagagagttggtcaacgtttccaagaacaaaaccAACTTATGATGCAACAGCTAAGACGTTAACCCCATATTTT | |
| | TGTACATAAAgttcattgtccaggactaatccagactttctctgaacagctctataatcttagtagtttcttccatcatttca | |
| | atcgttagcttcgaaacatcactgtcttcatcgttatagatgatggcatttgtaaacatgatctgtagaactttagtcaattc | |
| | gtcaaaggtggttatctccccatttCTGCAGTGCTTGAGAATGGTCTTCAGATCTTGA | |
| 35 | ATGGCTAAACTCACCTCTGCTGTTCCAGTCCTGACTGCTCGTGATGTTGCTGGTGCTGTTGAGTTCTGGACTGATAGACTCGG | Zeocin |
| | TTTCTCCCGTGACTTCGTAGAGGACGACTTTGCCGGTGTTGTACGTGACGACGTTACCCTGTTCATCTCCGCAGTTCAGGACC | resistance |
| | AGGTTGTGCCAGACAACACTCTGGCATGGGTATGGGTTCGTGGTCTGGACGAACTGTACGCTGAGTGGTCTGAGGTCGTGTCT | |
| | ACCAACTTCCGTGATGCATCTGGTCCAGCTATGACCGAGATCGGTGAACAGCCCTGGGGTCGTGAGTTTGCACTGCGTGATCC | |
| | AGCTGGTAACTGCGTGCATTTCGTCGCAGAAGAGCAGGACTAA | |
| 36 | ATGGGTAAAGAGAAAACGCACGTCAGTCGTCCAAGATTGAACTCCAATATGGATGCAGACCTGTACGGTTACAAATGGGCTAG | G418/ |
| | AGATAACGTTGGACAATCTGGTGCAACTATATATAGATTGTATGGGAAGCCAGACGCACCAGAGTTGTTTCTAAAGCATGGGA | Kanamycin |
| | AAGGCTCTGTTGCTAATGATGTGACTGATGAAATGGTACGTTTGAATTGGCTAACAGAGTTTATGCCCTTGCCTACTATTAAG | resistance |
| | CATTTTATTOGTACTCCCGATGACGCTTGGTTGCTAACCACCGCAATTCCTGGTAAAACTGCCTTTCAAGTTCTGGAAGAATA | |
| | CCCAGATTCCGGTGAAAACATCGTTGACGCCTTGGCTGTTTTCCTGCGAAGACTTCACTCTATTCCCGTATGTAATTGTCCCT | |
| | TTAATTCAGACAGAGTTTTTAGATTGGCTCAGGCTCAATCTAGGATGAATAATGGTTTGGTTGATGCAAGTGACTTCGATGAC | |
| | GAAAGAAACGGTTGGCCTGTCGAGCAGGTGTGGAAGGAAATGCATAAGTTACTTCCATTTTCTCCTGATTCTGTTGTAACCCA | |
| | CGGTGATTTTTCCCTAGACAACCTTATATTCGATGAGGGCAAGTTGATTGGTTGTATTGACGTCGGCAGAGTGGGTATCGCCG | |
| | ATAGGTATCAAGATTTAGCAATACTGTGGAATTGTCTAGGAGAATTTTCACCCAGTCTGCAAAAGAGATTGTTCCAGAAATAC | |
| | GGAATTGACAACCCCGATATGAATAAGTTGCAGTITCATTTGATGTTGGACGAGTTCTTCTAA | |
| 37 | ATGGGAAAGAAACCAGAGCTGACCGCAACGAGTGTCGAAAAATTTCTTATTGAAAAATTTGATAGTGTGTCCGATTTAATGCA | Hygromycin |
| | GCTTAGTGAAGGCGAAGAGTCACGTGCTTTCTCATTCGACGTTGGTGGACGTGGCTACGTTTTGAGAGTTAATAGTTGTGCAG | resistance |
| | ATGGCTTTTATAAGGATCGTTATGTATACCGTCATTTTGCTAGTGCAGCCCTGCCAATCCCAGAGGTTTTAGATATAGGTGAG | |
| | TTTAGTGAGTCTCTTACTTATTGTATTAGTCGTAGAGCCCAAGGTGTTACCCTTCAGGATTTGCCAGAGACTGAGCTTCCTGC | |
| | TGTATTGCAACCTGTCGCTGAGGCTATGGACGCCATTGCCGCAGCAGATTTATCTCAAACGTCAGGTTTCGGCCCCTTCGGCC | |
| | CACAAGGCATCGGACAGTACACAACGTGGCGTGACTTTATCTGTGCCATCGCTGACCCTCATGTCTACCACTGGCAAACGGTC | |
| | ATGGATGACACGGTGTCCGCCTCTGTGGCCCAAGCATTGGATGAACTGATGCTTTGGGCTGAGGATTGTCCCGAAGTCCGTCA | |
| | CCTGGTTCACGCTGACTTCGGCTCCAACAATGTTTTGACCGACAATGGCCGTATCACCGCTGTCATCGACTGGTCTGAGGCAA | |
| | TGTTTGGCGACTCTCAGTATGAAGTCGCCAATATATTTTTTTGGAGACCCTGGTTGGCATGCATGGAACAGCAAACTCGTTAC | |
| | TTTGAAAGACGTCATCCAGAGTTAGCTGGTAGTCCACGTCTGCGTGCTTACATGTTGCGTATCGGCTTAGACCAACTGTATCA | |
| | GTCACTTGTCGATGGTAACTTTGATGACGCAGCATGGGCACAAGGACGTTGTGACGCTATTGTACGTTCAGGTGCAGGCACGG | |
| | TCGGCCGTACACAAATTGCACGTAGAAGTGCAGCAGTCTGGACCGATGGTTGTGTTGAGGTCCTTGCAGATTCAGGAAATAGA | |
| | CGTCCATCTACTCGTCCTCGTGCTAAGGAATAA | |
| 38 | GGAATTGTGAGCGGATAACAATTCC | LacOperator |
| 39 | ATGGCCAATTTACTGACCGTACACCAAAATTTGCCTGCATTACCGGTCGATGCAACGAGTGATGAGGTTCGCAAGAACCTGAT | Cre |
| | GGACATGTTCAGGGATCGCCAGGCGTTTTCTGAGCATACCTGGAAAATGCTTCTGTCCGTTTGCCGGTCGTGGGCGGCATGGT | recombinase |
| | GCAAGTTGAATAACCGGAAATGGTTTCCCGCAGAACCTGAAGATGTTCGCGATTATCTTCTATATCTTCAGGCGCGCGGTCTG | |
| | GCAGTAAAAACTATCCAGCAACATTTGGGCCAGCTAAACATGCTTCATCGTCGGTCCGGGCTGCCACGACCAAGTGACAGCAA | |
| | TGCTGTTTCACTGGTTATGCGGCGCATCCGAAAAGAAAACGTTGATGCCGGTGAACGTGCAAAACAGGCTCTAGCGTTCGAAC | |
| | GCACTGATTTCGACCAGGTTCGTTCACTCATGGAAAATAGCGATCGCTGCCAGGATATACGTAATCTGGCATTTCTGGGGATT | |
| | GCTTATAACACCCTGTTACGTATAGCCGAAATTGCCAGGATCAGGGTTAAAGATATCTCACGTACTGACGGTGGGAGAATGTT | |
| | AATCCATATTGGCAGAACGAAAACGCTGGTTAGCACCGCAGGTGTAGAGAAGGCACTTAGCCTGGGGGTAACTAAACTGGTCG | |
| | AGCGATGGATTTCCGTCTCTGGTGTAGCTGATGATCCGAATAACTACCTGTTTTGCCGGGTCAGAAAAAATGGTGTTGCCGCG | |
| | CCATCTGCCACCAGCCAGCTATCAACTCGCGCCCTGGAAGGGATTTTTGAAGCAACTCATCGATTGATTTACGGCGCTAAGGA | |
| | TGACTCTGGTCAGAGATACCTGGCCTGGTCTGGACACAGTGCCCGTGTCGGAGCCGCGCGAGATATGGCCCGCGCTGGAGTTT | |
| | CAATACCGGAGATCATGCAAGCTGGTGGCTGGACCAATGTAAATATTGTCATGAACTATATCCGTAACCTGGATAGTGAAACA | |
| | GGGGCAATGGTGCGCCTGCTGGAAGATGGCGATTAA | |
| 40 | ATAACTTCGTATAATGTATGCTATACGAACGGTA | lox71 |
| | | (reverse |
| | | complement) |
| 41 | TACCGTTCGTATAATGTATGCTATACGAAGTTAT | lox66 |
| | | (reverse |
| | | complement) |
| 42 | CCCGTAGAAAAGATCAAAGGATCTTCTTGAGATCCTTTTTTTCTGCGCGTAATCTGCTGCTTGCAAACAAAAAAACCACCGCT | pUC origin |
| | ACCAGCGGTGGTTTGTTTGCCGGATCAAGAGCTACCAACTCTTTTTCCGAAGGTAACTGGCTTCAGCAGAGCGCAGATACCAA | of |
| | ATACTGTTCTTCTAGTGTAGCCGTAGTTAGGCCACCACTTCAAGAACTCTGTAGCACCGCCTACATACCTCGCTCTGCTAATC | replication |
| | CTGTTACCAGTGGCTGCTGCCAGTGGCGATAAGTCGTGTCTTACCGGGTTGGACTCAAGACGATAGTTACCGGATAAGGCGCA | |
| | GCGGTCGGGCTGAACGGGGGGTTCGTGCACACAGCCCAGCTTGGAGCGAACGACCTACACCGAACTGAGATACCTACAGCGTG | |
| | AGCTATGAGAAAGCGCCACGCTTCCCGAAGGGAGAAAGGCGGACAGGTATCCGGTAAGGGGCAGGGTCGGAACAGGAGAGCGC | |
| | ACGAGGGAGCTTCCAGGGGGAAACGCCTGGTATCTTTATAGTCCTGTCGGGTTTCGCCACCTCTGACTTGAGCGTCGATTTTT | |
| | GTGATGCTCGTCAGGGGGGCGGAGCCTATGGAAAAACGCCAGCAACGCGGCCTTTTTACGGTTCCTGGCCTTTTGCTGGCCTT | |
| | TTGCTCACA | |

### High-Titer Production of Recombinant Proteins

The methods herein provide for improved high-titer production of recombinant protein from engineered host cells in a high-volume growth format, such as in a fermentation tank.

In some embodiments, the methods herein include heterologous protein production from engineered host cells in a large-scale growth settings at culture volumes of greater than about 1, 2, 3, 5, 10, 20, 50, 100, 500, 1000 liters and over time periods such as 1, 2, 3, 4, 5, 6, 7, 8, 9, 10 or more than 10 days. The systems and methods herein provide titers of the desired protein under fermentation conditions of at least 1, 2, 3, 4, 5, 6, 7, 8, 9, 0, 11, 12, 13, 14, 15, 16, 17, 18, 19, 20, 21, 22, 23, 24, 25, 26, 27, 28, 29, 30, 50 g protein/liter of culture media. The desired titers of the heterologous protein can be reached over time periods such as 6 hours, 12 hours, 18 hours, 24 hours, 48 hours or 72 hours. In some cases, the desired titers of the heterologous protein under fermentation conditions can be reached over time periods such as 1, 2, 3, 4, 5, 6, 7, 8, 9, 10 or more than 10 days. In some embodiments, such titers are the amounts of secreted desired protein from the fermentation culture. In some embodiments, such titers are the amounts of total desired protein (intracellular and extracellular) from the fermentation culture. In some embodiments, such titers are the amounts of secreted protein from the fermentation culture.

In some embodiments, the methods herein include heterologous recombinant protein production from engineered host cells reaching culture densities of up to 10 grams of cells per liter of culture media, 30 g/L, 40 g/L, 50 g/L, 70 g/L, 100 g/L or 150 g/L. In some embodiments, the methods herein include heterologous recombinant protein production from engineered host cells reaching cell densities of up to 100 g dry cell weight/L, 150 g dry cell weight/L or 200 g dry cell weight/L.

In some embodiments, the methods herein include heterologous recombinant protein production from engineered host cells at a titer of at least 3 g/L of culture media. In some embodiments, the methods herein include heterologous recombinant protein production from engineered host cells at a titer of at least 5 g/L, 8 g/L, 10 g/L, 15 g/L, 20 g/L, 25 g/L, 30 g/L, 35 g/L, 40 g/L, 50 g/L, 60 g/L, 70 g/L, 80 g/L, 100 g/L or above.

### Fermentation Conditions

The methods herein provide for fermentation conditions that can provide improved high-titer production of heterologous proteins from engineered host cells in a high-volume growth format, such as in a fermentation tank. Yeast strain glycerol stocks are thawed and inoculated at a 0.2% inoculum ratio in baffled shake flasks containing BMDY media (BMDY is BMGY where the glycerol. ‘G’, has been replaced with glucose/dextrose, ‘D’, Pichia Easy Select Manual, Thermo Fisher). Shake flasks are left to incubate at 30 C and 250 rpm for 26 hrs. Shake flask cultures are then transferred at a 10% ratio to bioreactors containing BSM (basal salt medium), glucose, and trace metals (Pichia Fermentation Process Guidelines, Thermo Fisher).

The bioreactor fermentation is divided into three phases. During phase 1, the culture may be grown for 24 hrs until all glucose is consumed. During phase 2, the culture may be fed glucose at a glucose-limiting rate for 12 hours. In phase 3, the culture may be induced by continuously feeding a co-feed of glucose and methanol for 96 hours.

In one embodiment, the invention provides a method of improving the volumetric productivity of a recombinant protein of interest from host cells under fermentation culture conditions. In embodiments, the invention provides a cell culture medium optimized for use in a methanol inducible fermentation system (e.g., under the control of the AOX1 promoter) for the production of a recombinant protein of interest in yeast host cells using a fed-batch fermentation process. In embodiments, the invention provides a cell culture medium optimized for use in a methanol inducible fermentation system (e.g., under the control of the AOX1 promoter) for the production of a recombinant protein of interest in yeast host cells using a continuous fermentation process. In some cases, the host cell is a yeast cell.

In embodiments, the method comprises a) providing a glycerol fed yeast host cell culture comprising host cells that are engineered as described elsewhere in this application b) providing a methanol fed medium, and optionally an osmoprotectant, and c) inducing the yeast host cells under fermentation conditions to allow expression of the recombinant protein wherein the volumetric productivity of the protein of interest is higher than at least 1 g/L. As used herein the term “volumetric productivity” means the amount of target recombinant protein per unit volume of culture (g/L). In some embodiments, optimization of fermentation conditions can be used to improve the volumetric productivity of the host cells engineered as described herein by 20%, 25%, 30%, 35%, 40%, 45%, 50%, 55%, 60%, 65%, 70%, 75%, 80%, 85%, 90%, 95% or more than 100%.

In some cases, a seed culture of the host cell engineered as described elsewhere in this application is inoculated into a starter culture composed of suitable culture medium. In some cases, the medium is BSGY medium. In some cases, the medium is BMDY media. In some cases, the volume of the starter culture medium is up to 200 ml, up to 300 ml, or up to 500 ml. In some cases, the starter culture is incubated at a temperature of 24° C., 25° C., 26° C., 27° C., 28° C., 29° C., 30° C., 31° C. or 32° C. In some cases, the starter culture is incubated for up to 6 hours, 12 hours, up to 24 hours, up to 36 hours or up to 48 hours. In some cases, the starter culture is shaken during incubation at 100 rpm, 200 rpm, 300 rpm, 500 rpm or 600 rpm. In some cases, a bioreactor system providing fermentation conditions for cultivation of host cells, is inoculated with a volumetric ratio of seed to initial fermentation medium of up to 3%, up to 5%, up to 100%, up to 15% or up to 20%. In some cases, the initial fermentation medium is BSGY medium. In some cases, the initial fermentation medium is BSM medium (basal salt medium). In some cases, the initial fermentation medium contains glucose and trace metals.

In embodiments, methanol inducible fermentation systems based on the AOX1 or other methanol-inducible promoter when present in an expression cassette can involve use of glycerol as a substrate for biomass growth, followed by a methanol feed for induction. In embodiments, cultivation of host cells under fermentation conditions involves a multistage fermentation process. In embodiments, the multistage process is a batch fed process. In embodiments, the initial stage can include a glucose fed phase where the cells are cultured in a glucose-containing medium to accumulate biomass. In some cases, the initial stage can include a glycerol fed phase where the cells are cultured in a glycerol-containing medium to accumulate biomass. These methods are especially useful in the context of a MutS strain.

In embodiments, in the next stage, the cells can be fed glucose at a rate limiting rate to prepare for induction phase. In embodiments, the rate limiting feeding rate of glucose can range from up to 0.005 g/l, up to 0.05 g/1, or up to per hour of specific growth rate. In some cases, glucose can be fed for up to 8 hours, up to 10 hours, up to 14 hours, up to 16 hours, up to 20 hours, or up to 24 hours, up to 30 hours, up to 36 hours, up to 40 hours, or up to 48 hours. In some cases, host cells can be fed with glycerol instead of before methanol induction.

In some cases, the methanol induction phase can be preceded by a starvation phase. In some cases, the starvation phase before induction can last for 30 minutes, up to 60 minutes, up to 90 minutes, up to 120 minutes, up to 150 minutes, up to 180 minutes, up to 4 hours, up to 6 hours or up to 8 hours.

In some cases, methanol feed rate can be optimized to improve production of recombinant protein production in host cells. In some cases, methanol feeding regimes, for example, maintaining a fixed methanol concentration (Damasceno et al, 2004), controlling dissolved oxygen concentration with methanol feed rate (Charoenrat et al, 2005), carbon limited feed strategies (Zhang et al, 2000) as well as mixed carbon source feeds (Ramon et al, 2007) can be used for increasing the rate of production of heterologous protein from engineered host cells. In some cases, methanol can be continuously fed at a constant rate. In some cases, the methanol feed rate can be up to 0.5 g/L/h, up to 0.7 g/L/h, 0.8 g/L/h, 0.9 g/L/h, 1.1 g/L/h, 1.3 g/L/h, 1.5 g/L/h, 1.6 g/L/h, 1.8 g/L/h, 1.9 g/L/h, 2.1 g/L/h, 2.4 g/L/h, 2.6 g/L/h, 2.7 g/L/h, 2.9 g/L/h, 3.1 g/L/h, 3.3 g/L/h, 3.5 g/L/h, 3.7 g/L/h, 3.9 g/L/h, 4.5 g/L/h or 5.0 g/L/h. In some cases, methanol can be fed at an exponential rate. In some cases, methanol can be added as a periodic bolus. In some case, host cells are co-fed glucose along with methanol. In some cases, the glucose feeding rate can be up to 0.5 g/L/h, up to 0.7 g/L/h, 0.8 g/L/h, 0.9 g/L/h, 1.1 g/L/h, 1.3 g/L/h, 1.5 g/L/h, 1.6 g/L/h, 1.8 g/L/h, 1.9 g/Uh, 2.1 g/L/h, 2.4 g/L/h, 2.6 g/L/h, 2.7 g/L/h, 2.9 g/L/h, 3.1 g/L/h, 3.3 g/L/h, 3.5 g/L/h, 3.7 g/L/h, 3.9 g/L/h, 4.5 g/L/h or 5.0 g/L/h.

In some cases, the length of methanol induction phase can be up to 1 day, up to 2 day, up to 3 days, up to 4 days, up to 5 days, up to 6 days, up to 7 days, up to 8 days, up to 9 days, or up to 10 days. In some cases, the length of methanol induction phase can be at least 1 day, at least 2 day, at least 3 days, at least 4 days, at least 5 days, at least 6 days, at least 7 days, at least 8 days, at least 9 days, or at least 10 days.

Suitable culture media can be designed to provide pure carbon sources. In some cases, the media can optionally provide biotin, salts trace elements and water. In some cases, the carbon source for the host cells can be selected from glucose, fucose, mannose, sorbose, or glycerol, sorbitol. In some cases, the medium can be BSGY, BMMY, MD, or YPD medium. In some cases, the medium composition can influence heterologous protein expression in host cells by affecting cell growth and viability or altering the secretion of extracellular proteases. In some cases, sorbitol or betaine can be added to culture media to increase production of the heterologous recombinant protein. In embodiments, the addition of an organic nitrogen source (e.g., a mixture of yeast extract and peptone) to a fed-batch culture system can be used to increase heterologous protein production in host yeast cells.

Cell wall integrity of the host cells can affect the production yield of the heterologous protein. In embodiments, improved culture conditions utilizing optimized media and fermentation conditions can be designed to improve the cell well integrity of the engineered Pichia strains. For example, in embodiments, the fermentation medium can comprise a basal medium supplemented with a non-fermentable sugar or a non-fermentable sugar alcohol as an osmoprotectant. In particular embodiments, the osmoprotectant can be selected from maltose, sorbose, ribose, maltitol, myo-inositol, mellibiose, and quinic acid. In some cases, glycerol, arabitol, glycine betaine, sorbitol or trehalose can be utilized for modulating cellular osmotic pressure under osmotic stress conditions. The osmoprotectants can be added to any suitable basal medium. In particular embodiments the osmoprotectant can be added in addition to other media supplements, including, but no limited to mixes comprising amino acids, vitamins, trace metals or basal salts. In embodiments, the inclusion of the osmoprotectant can be maintained through the glycerol feeding phase, the methanol induction phase or both.

In embodiments, the osmoprotectant is present at concentration of about 15 g/L, about 25 g/L, about 35 g/L, about 50 g/L, about 75 g/L or about 100 g/L. In embodiments, the presence of the osmoprotectant in the batch media increases and maintains the osmolality of the batch media at more than about 50 mOsm/kg, more than about 100 mOsm/kg, more than about 200 mOsm/kg, more than about 500 mOsm/kg, more than about 700 mOsm/kg, more than 1000 mOsm/kg, or more than about 1500 mOsm/kg. In embodiments, increased osmolality is maintained from about 24 hours to about 48 hours, to about 80 hours to about 110 hours or until completion of the methanol induction phase (e.g., ranging from about 24 to about 150 hours). In some cases, the increased osmolality is maintained through the methanol feeding phase.

In some cases, cultivation parameters e.g., pH, temperature or dissolved oxygen can be optimized to improve production of recombinant protein production in host cells. In some cases, the cultivation temperature conditions can be at least 24° C., 24.1° C., 24.2° C., 24.5° C., 24. 8° C., 26.0° C., 26.3° C., 26.5° C., 26.8° C., 27.0° C., 27.2° C., 27.5° C., 27.8° C., 29.0° C., 29.3° C., 29.5° C., 29.8° C., 30.0° C., 30.3° C., 30.5° C., 30.7° C., 31° C., 31.3° C., 31.5° C. 31.7° C., 31.9° C., 32.3° C., 32.6° C., 32.8° C., 33.0° C., 33.1° C., 33.5° C., 33.6° C. or 34.0° C. In some cases, the pH of the fermentation cultivation conditions can be up to 6.2, up to 6. 4, up to 6.6, up to 6.7, up to 6.8, up to 6.9, up to 7.0, up to 7.1, up to 7.3, up to 7.5, up to 7.8, up to 7.9, or up to 8.0. In some cases, dissolved oxygen levels can be maintained at up to 15%, up to 17%, up to 20%, up to 22%, up to 25%, up to 27%, up to 30%, up to 32% or up to 35% of saturation.

### Proteins

In some embodiments, the methods provided herein can be used for the production of therapeutic proteins in a large-scale fermentation setting. In some embodiments, the methods provided herein can be used for the production of enzymes or antibodies in a large-scale fermentation setting. In some embodiments, the methods provided herein can be used for the production of plant-derived food-related proteins in a large-scale fermentation setting. In some embodiments, the methods provided herein can be used for the production of animal-derived food-related proteins in a large-scale fermentation setting. In some cases, the animal or plant-derived protein is an enzyme, such as used in processing and/or production of food and/or beverage ingredients and products. Some examples of enzymes including trypsin, chymotrypsin, pepsin and pre- and pre-pro-forms of such enzymes. In some cases, the animal or plant protein is a nutritive protein such as a protein that holds or binds to a vitamin or mineral (e.g., an iron-binding protein or heme binding protein), or a protein that provides a source of protein and/or particular amino acids.

In some embodiments, the methods provided herein can be used for the production of food proteins in a large-scale fermentation setting. In some cases, the food protein can be a plant protein. In some cases, the food protein can be an animal protein. In various cases, the food protein may be used as nutritional, dietary, digestive, supplements, such as in food products and feed products. In some embodiments, the animal protein can be an egg-related protein. Illustrative examples of such egg white proteins can be ovalbumin, ovomucoid, ovotransferrin, and lysozyme proteins. Other examples of egg-related proteins include ovomucin, ovoglobulin G2, ovoglobulin G3 and any combination thereof. Additional examples of egg-related proteins include ovoinhibitor, ovoglycoprotein, flavoprotein, ovomacroglobulin, ovostatin, cystatin, avidin, ovalbumin related protein X, ovalbumin related protein Y and any combination thereof.

In some cases, the protein produced using the systems and methods provided herein is post-translationally modified. Such modifications include glycosylation and phosphorylation. In some cases, the post-translational modification of the produced protein is the same or substantially similar to the natively produced protein. In some cases, the post-translational modification of the produced protein is altered as compared to the native source of the protein.

Food compositions can include the recombinant food proteins, e.g., recombinant ovomucoid, in an amount between 0.1% and 50% on a weight/weight (w/w) or weight/volume (w/v) basis. Recombinant proteins produced using the systems and methods herein, may be present in food compositions at or at least at 0.1%, 0.2%, 0.25%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45% or 50% on a weight/weight (w/w) or weight/volume (w/v) basis. Additionally, or alternatively, the concentration of recombinant proteins produced using the systems and methods herein, may be present in such food compositions is at most 70%, 60%, 50%, 40%, 30%, 20%, 15%, 10%, 5%, 4%, 3%, 2% or 1% on a w/w or w/v basis. In some embodiments, the recombinant protein in the food ingredient or food product can be at a concentration range of 0.1%-50%, 1%-30%, 0.1%-20%, 1%-10%, 0.1%-5%, 1%-5%, 0.1%-2%, 1%-2% or 0.1-1%.

In some embodiments, the methods provided herein can be used for the production of non-food proteins in a large-scale fermentation setting. In some cases, the non-food protein can be a protein suitable as a biopharmaceutical substance like an antibody or antibody fragment, growth factor, hormone, enzyme, vaccine, regulatory protein, receptor, cytokine, antigen-binding proteins, immune stimulatory proteins, scaffold binding protein, structural protein, lymphokine, adhesion molecule, membrane or transport protein, other polypeptides that can serve as an agonist or antagonist and/or have therapeutic or diagnostic use, or a protein which can be used for industrial or cosmetic applications. In some embodiments, a non-food protein that has medical or research applications may be produced, e.g., Adenosine deaminase, Alpha-galactosidase A, Alpha-L-iduronidase (rhIDU), Anti-thrombin III, Coagulant Factors (e.g., Coagulation factors VII, VIII, and IX), DNAseI, Domase alfa, Epidermal growth factor, Erythropoietin (EPO), Follicle stimulating hormone (FH), Glucagon, Glucocerebrosidase, Granulocyte colony stimulating factor (G-CSF), Granulocyte colony-stimulating factor (G-CSF), Granulocyte Macrophage Colony-Stimulating Factor (GM-CSF), Human growth hormone (HGH), Human serum albumin, Insulin, Insulin-like growth factor 1 (IGF-1), Interferon (IFN) (e.g., IFN alpha, IFN beta (e.g., Interferon beta-1b and Interferon-beta-1a), IFN gamma (e.g., gamma-1b interferon), IFN omega, and IFN tau), Interleukins (such as IL-1, IL-2, IL-3, IL-4, IL-5, IL-6, IL-7, IL-8, IL-9, IL-10, IL-1 1. IL-12, IL-13, IL-14, IL-15, IL-16, IL-17, and IL-18), Macrophage colony-stimulating factor (M-CSF), Monocyte chemoattractant protein-1 (MCP-1), N-acetylgalactosamine-4-sulfatase (rhASB), Nerve growth factor (NGF), Platelet-derived growth factor (PDGF), Protein C, Rasburicase, Tissue plasminogen activator (TPA), TRAIL, Tumor necrosis factor (TNF) (e.g., TNF alpha and TNF beta), or Vascular endothelial growth factor (VEGF). Non-food proteins may be enzymes which can be used for industrial application, such as in the manufacturing of a detergent, starch, fuel, textile, pulp and paper, oil, personal care products, or such as for baking, organic synthesis, and the like. Examples of such enzymes include protease, amylase, lipase, mannanase and cellulose for stain removal and cleaning; pullulanase amylase and amyloglucosidase for starch liquefaction and saccharification; glucose isomerase for glucose to fructose conversion; cyclodextrin-glycosyltransferase for cyclodextrin production; xylanase for viscosity reduction in fuel and starch; amylase, xylanase, lipase, phospholipase, glucose, oxidase, lipoxygenase, transglutaminase for dough stability and conditioning in baking; cellulase in textile manufacturing for denim finishing and cotton softening; amylase for de-sizing of textile; pectate lyase for scouring; catalase for bleach termination; laccase for bleaching; peroxidase for excess dye removal; lipase, protease, amylase, xylanase, cellulose, in pulp and paper production; lipase for transesterification and phospholipase for de-gumming in fat processing fats and oils; lipase for resolution of chiral alcohols and amides in organic synthesis; acylase for synthesis of semisynthetic penicillin, nitrilase for the synthesis of enantiopure carboxylic acids; protease and lipase for leather production; amyloglucosidase, glucose oxidase, and peroxidase for the making personal care products. The non-food protein may be an enzyme or a protein used in cosmetic product, such as collagen, elastin, and keratin. In some cases, the non-food protein is a eukaryotic protein or a biologically active fragment thereof. In various embodiments, the non-food protein is immunoglobulin or an immunoglobulin fragment such as a Fc fragment or a Fab fragment.

Any protein that can be expressed by a yeast cell may be used in the methods, engineered host cells, and kits of the present disclosure. Moreover, an advantage of the present disclosure is that it may be adapted as the need for future recombinant protein expression as these needs are discovered. More specifically, once a protein having a dietary, medical, industrial, cosmetic, or research has been identified and determined to be in need of recombinant expression, this protein's gene sequence can be used in the methods, engineered host cells, and kits of the present disclosure to express sufficient quantities of the protein In other words, the present disclosure is not limited by the illustrative genes and proteins recited herein.

### EXAMPLES

The following examples are included for illustrative purposes only and are not intended to limit the scope of the invention.

### Example 1: Construction of a Host Cell with Multi-Copy Expression Cassettes

Four different multicopy constructs comprising multiple expression constructs were mixed together and transformed into a naïve MutS K. phaffii strain (similar to BG11) to create a library of zeocin resistant (ZeoR) transformants.

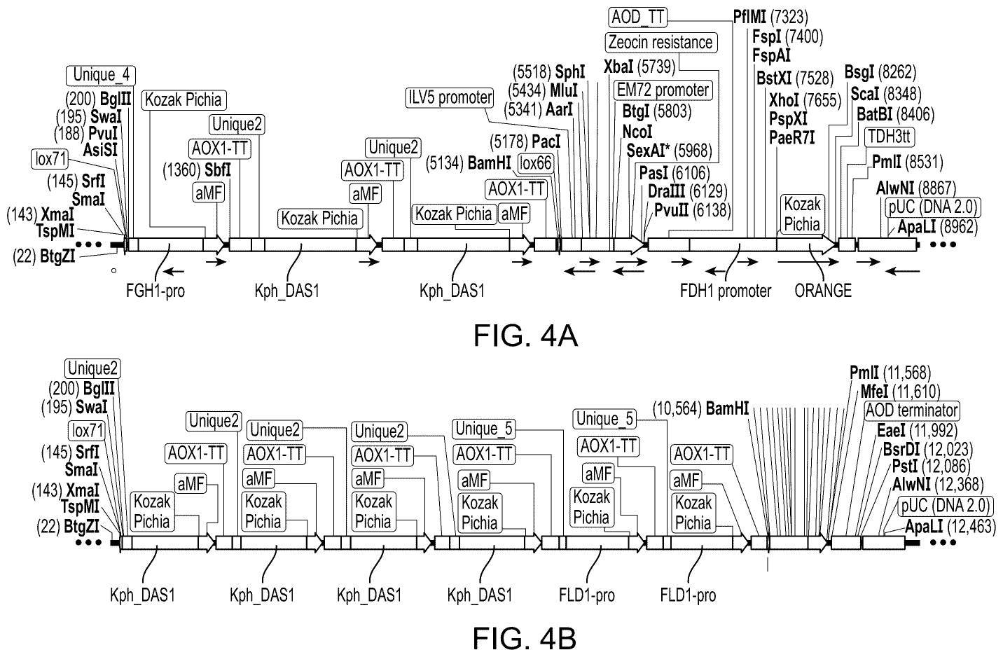

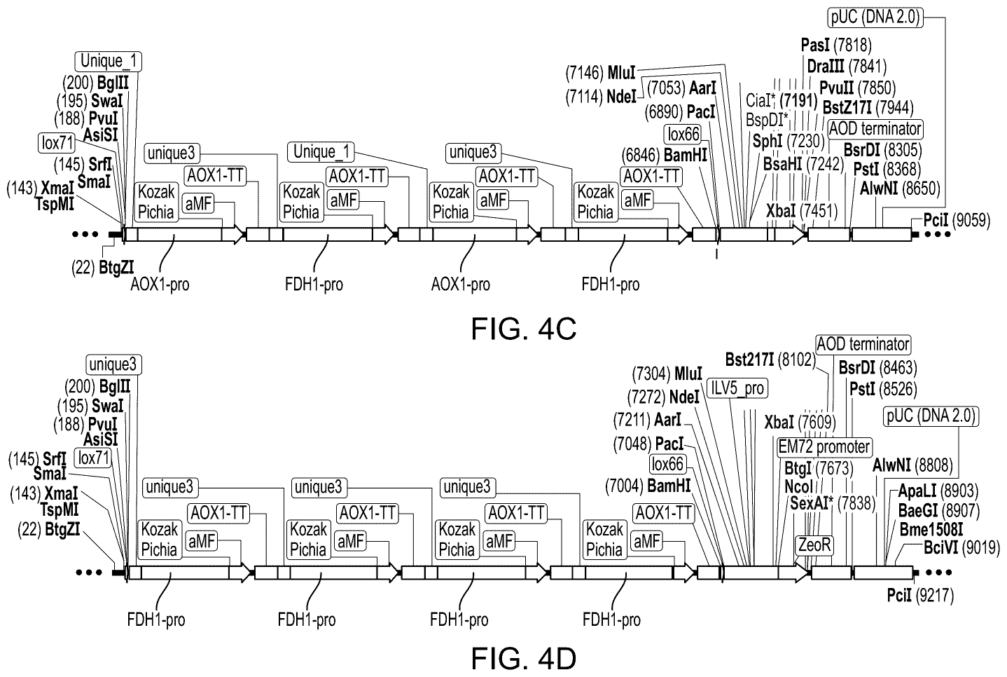

Long stokes shift (LSS) refers to the separation of excitation and emission spectra. LSS proteins have an identifiable separation in spectra. The expression constructs included the following plasmids: 1) 3 copy construct: contained two DAS1 promoters and one FGH1 promoter expression constructs (FIG. 4A); additionally contained FDH1-LSS-mOrange (long stokes shift) in the vector backbone; 2) 6 copy construct: contained four DAS1 and two FLD1 promoter expression constructs (FIG. 4B); 3) 4 copy construct: contained two AOX1 and two FDH1 promoter expression constructs (FIG. 4C); 4) 4 copy construction construct. Contained four FDH1 expression constructs (FIG. 4D). All constructs had terminators selected from AOX1 terminator sequence (Aox1tt) or AOD terminator sequence. Alpha mating factor sequence (αMF) was also placed as a signal sequence operably linked to each promoter.

A colony forming unit (cfu) assay determined that this host cell population possessed ˜50,000 total transformants. Ten percent (˜5000 cfu) of the transformants were subjected to competent cell preparation under zeocin selection.

### Example 2: Transfection of mCherry Coding Sequences in Engineered Host Cells

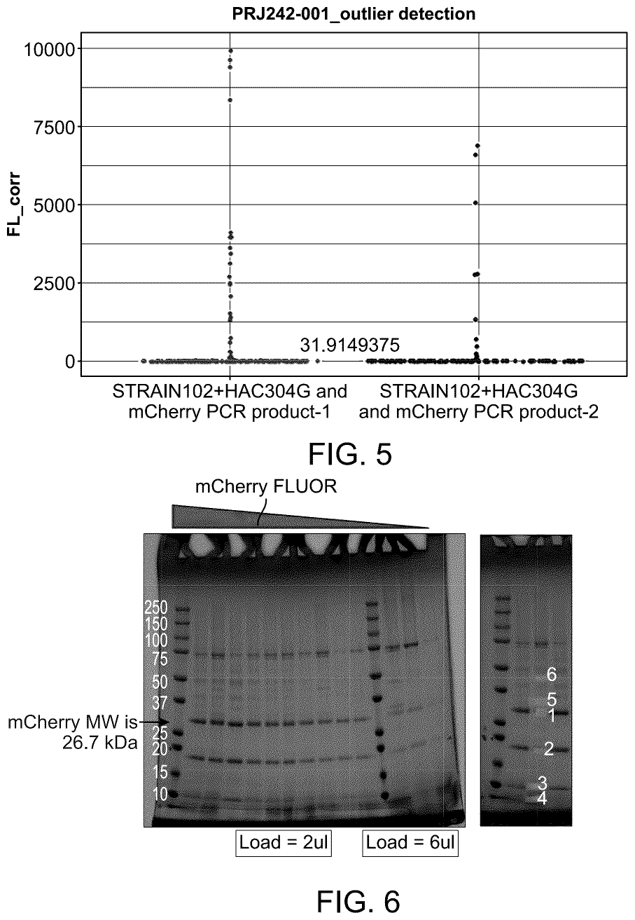

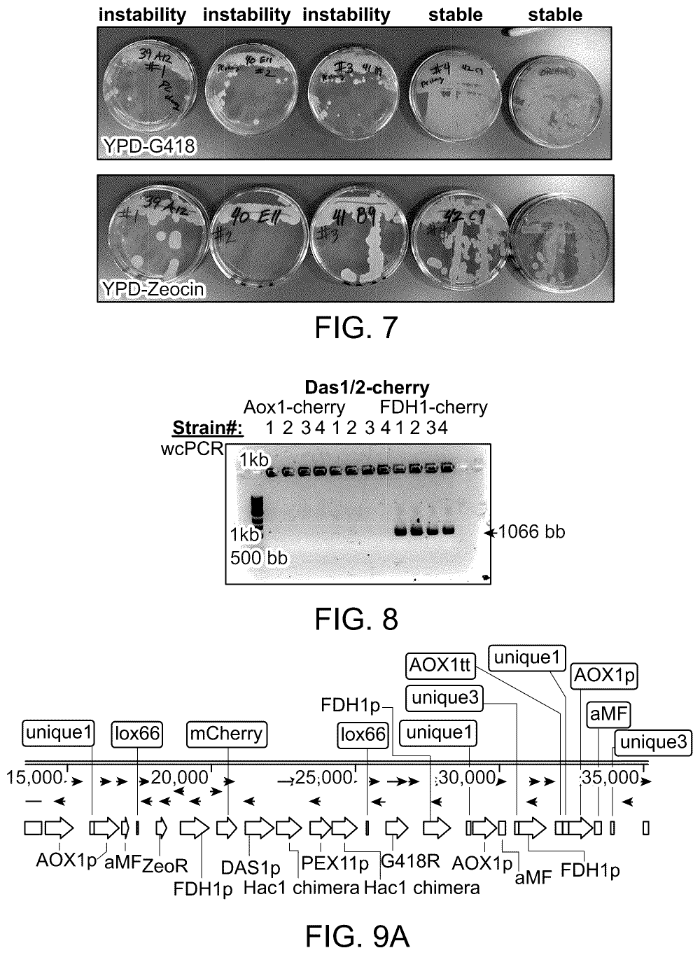

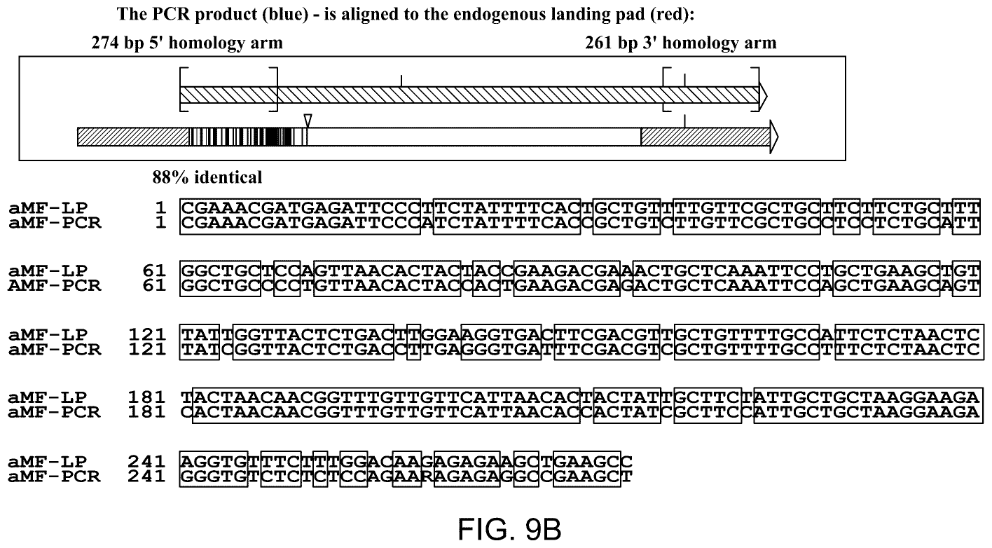

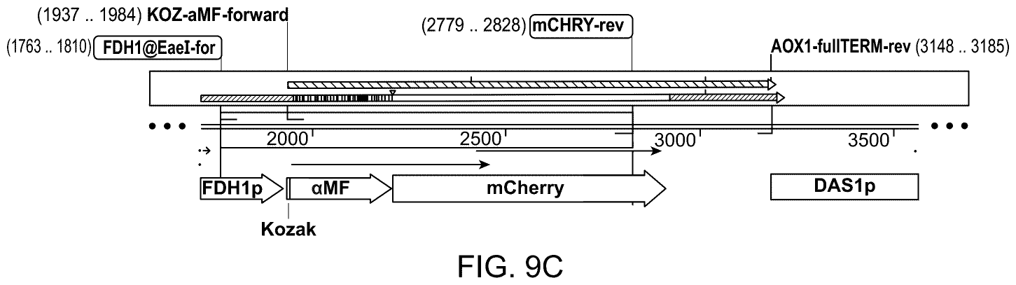

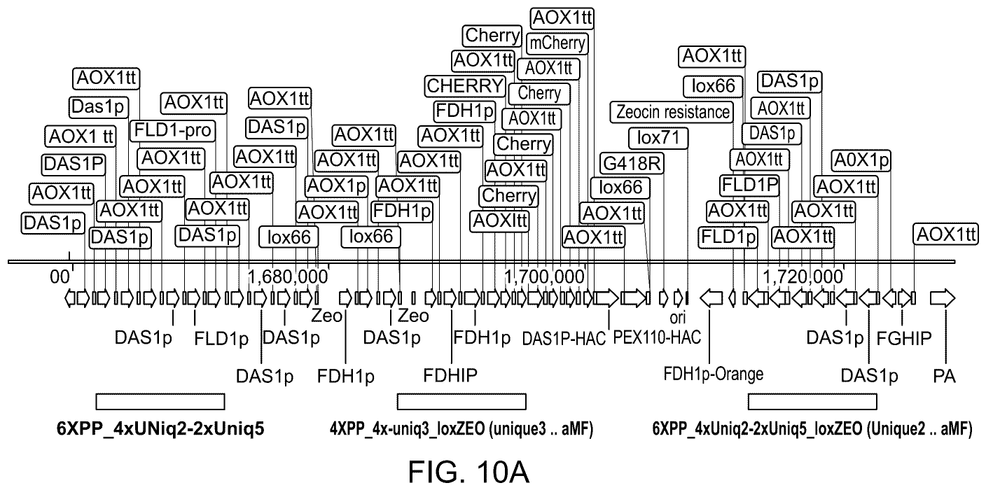

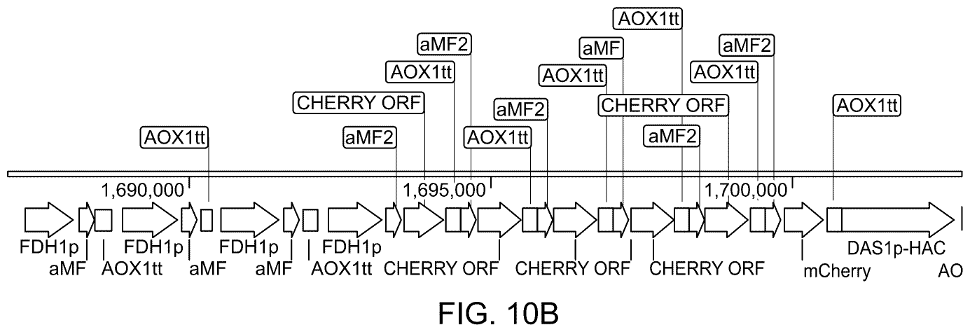

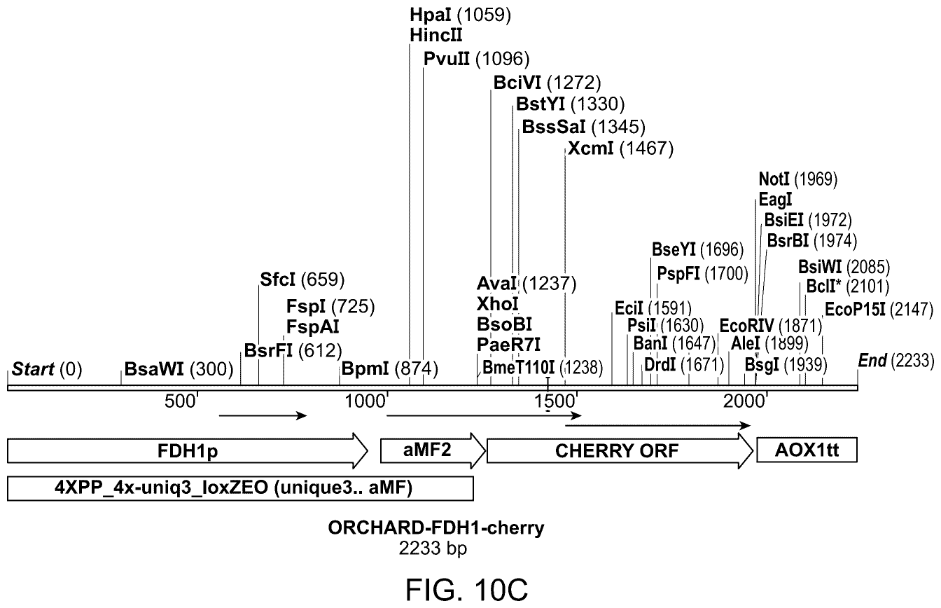

Using PCR combined with the high-fidelity DNA polymerase, Q5, a cDNA encoding αMF-mCherry through the 3′ untranslated aox1 transcriptional terminator (aox1 TT) was amplified, then co-transformed with a linearized selectable marker that encodes three copies of the transcriptional activator HAC1p [G418 co-transformation] for selection on yeast extract peptone dextrose (YPD) with 2 mg/ml geneticin (G418). G418 resistant colonies were isolated into deep-well plates and subjected to a 7-day time course under methanol induction. Cell-free supernatants were analyzed by fluorescence spectroscopy [λEx 587 nm, λEm 610 nm] (as shown in FIG. 5) and SDS-PAGE (as shown in FIG. 6). Several mCherry secreting clones were further subjected to a genetic marker stability test (FIG. 7) and purification using single colony isolations on YPD-G418 (2 mg/ml) agar plates. Diagnostic PCR with primers that anneal to the unique barcodes in the expression constructs intrinsic to the strain and a specific target gene sequence revealed the presence of novel DNA molecules created in vivo (FIG. 8). Two mCherry secreting clones were further subjected to full genome sequencing (FIGS. 9A-C and FIGS. 10A-C).

Two independent electroporations were conducted followed by colony-picking into four %-deep well (DW) plates. Out of a total of 352 clones examined for mCherry secretion, ˜198 gave a positive signal for the presence of mCherry fluorescent protein in cell-free supernatants giving a conversion to mCherry+ as 56%, 55 of the 352 colonies analyzed for mCherry secretion were intracellularly fluorescent LSSmOrange strains, indicating the presence of expression constructs as shown in FIGS. 4A-D integrated into approximately 15% of the host cell's library.

The offset gel panel in FIG. 6 indicates the six separate excised protein bands that were subjected to mass spectrometry characterization: band #1 is full-length mCherry; band #2 is mCherry truncated on amino-terminus; band #3 is full-length mCherry and some Kar2 (aka “binding protein” or BiP) and protein disulfide isomerase (PDI); band #4 is a truncated form of mCherry with some Kar2; band #5 is Kar2, mCherry and PDI; band #6 is also Kar2, PDI and mCherry. SDS-PAGE lane assignments are provided in Table 6 below:

TABLE 6
Lane assignments
HTS1S7: GEL#lO

| | HTS Primary | HTS Primary | | |
|---|---|---|---|---|
| LANE: | P_{——} | P_{——} | Coordinate | FL_corr |
| 1 | LADDER | | | cherry |
| 2 | DW4040 | DW4040-059 | E11 | 10003.1485 |
| 3 | DW4039 | DW4039-012 | A12 | 9706.7865 |
| 4 | DW4040 | DW4040.003 | A3 | 9460.7155 |
| 5 | DW4040 | DW4040-037 | D1 | 8417.6125 |
| 6 | DW4041 | DW4041-021 | B9 | 6951.844 |
| 7 | DW4042 | DW4042-033 | C9 | 6653.5165 |
| 8 | DW4041 | DW4041-033 | C9 | 5108.78 |
| 9 | DW4040 | DW4040-082 | G10 | 4141.6225 |
| 10 | DW4041 | DW4041-052 | E4 | 4004.692 |
| 11 | DW4039 | DW4039-094 | H10 | 4002.8465 |
| 12 | LADDER | | | |
| 13 | DW4039 | DW4039.074 | G2 | 1419.2285 |
| 14 | DW4042 | DW4042-086 | H2 | 1340.0185 |
| 15 | DW4040 | DW4040-044 | D8 | 1331.5605 |

A genetic marker stability test is demonstrated in FIG. 7 which was acquired by streaking for single colonies on selective medias. Three strains marked “instability” show poor growth and colony size heterogeneity when grown on YPD-G418 (selectable marker on the hac1 construct). In contrast, the two illustrative strains (of FIGS. 9A-C and FIGS. 10A-C) showed confluent growth on both medias and produced healthy and homogenously sized colonies.

As the four multicopy expression constructs could potentially drive mCherry expression from any or all of the five (AOX1, DAS1, FDH1, FGH1, or FLD1) promoters encoded on the individual expression cassettes (see above), diagnostic PCR was conducted to identify the presence of promoter-mCherry expressing constructs. FDH1 expression cassettes predominate the expression construct mixture, the FDH1-mCherry PCR product was amplified from all four strains with this diagnostic primer pair (see FIGS. 8 and 9A-C below). Neither AOX1- or DAS1-mCherry novel DNAs were detected in the selected host cells.

Two mCherry illustrative secreting clones, the latter of which expresses an intracellular fluorescent orange, were further subjected to full genomic sequencing using a MinION long-read sequencer (oxford Nanopore Technologies).

The illustrative strain of FIGS. 9A-C, is a conglomeration of, about two expression construct multicopies (see FIGS. 4A-D, construct #3 above) integrated into the endogenous FDH1 locus. The subsequent transformation with the 3 copy hac1 and unmarked, promoter-less mCherry PCR product resulted in the integration of the hac1 G418 plasmids into the array formed of expression constructs along with a single copy of mCherry, now under control of the FDH1 promoter (see FIG. 9A).

The endogenous αMF that resides within all expression constructs (“αMF-LP”) is 88% identical to the incoming αMF that is fused to mCherry (aka “αMF-PCR”). Despite these slight variations, PCR product-expression construct fusions were observed and the strains analyzed successfully secrete mCherry protein.

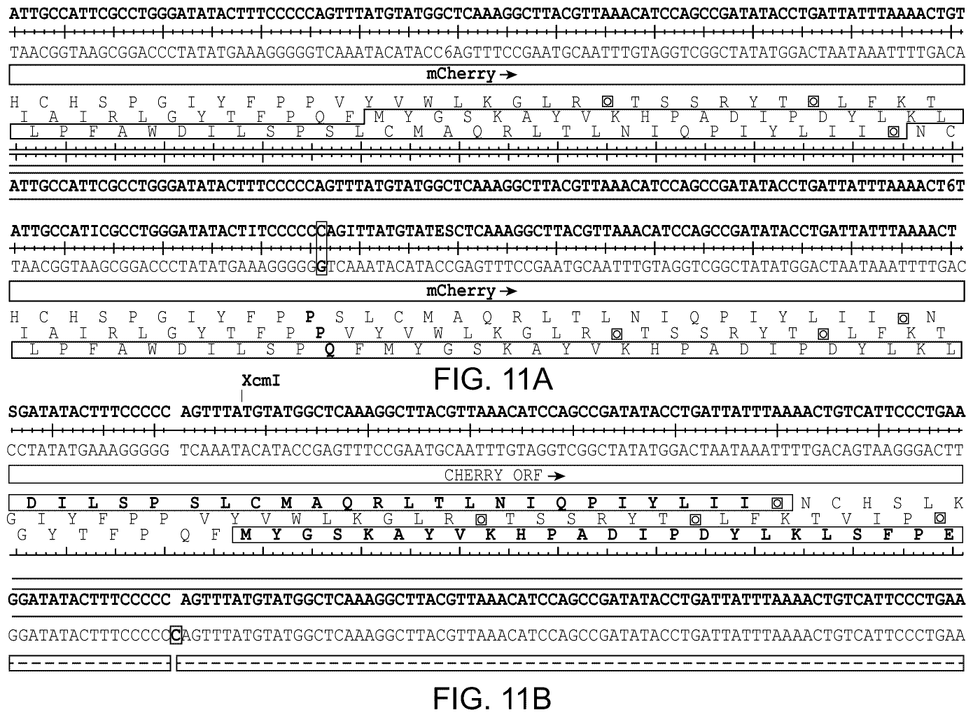

The light blue highlighted region in FIG. 9C shows the predicted 1066 bp PCR product that is amplified with the PCR primer pair: “FDH1@EaeI-for”/“mCHRY-rev”. The genomic sequence of the illustrative strain of FIGS. 9A-C also shows a frameshift, however as the strain produces copious amounts of secreted, fluorescent mCherry protein this mutation is unlikely. Moreover, as the genomic sequence of this strain shows a frameshift in the same region, the likelihood of a base-calling error is expected to be the reason for the shift (see FIGS. 11A-B).

The illustrative strain of FIGS. 10A-C makes fluorescent LSSmOrange intracellularly and secretes fluorescent mCherry protein. The genomic DNA sequence of this strain revealed the presence of a large expression construct array on chromosome three that possesses approximately twenty expression constructs laid sequentially. The subsequent transformation resulted in six copies of the mCherry PCR product in tandem, however only one is predicted to be under the control of an inducible promoter (FDH1; see FIGS. 10B and 10C). Similar to the outcome with the illustrative strain of FIGS. 10A-C, the 3 copy hac1 plasmid construct can also be found embedded within this expression construct array.

Similar to the illustrative strain of FIGS. 9A-C, the mCherry ORF is fused in-frame to the endogenous FDH1 expression cassette. A similar frameshift mutation (see FIGS. 11A-B) is also observed in the genomic DNA sequence; most likely due to a base-calling error in a polycytosine (C) region immediately preceding the frameshifts observed.

### Example 3: Transfection of Egg White Protein Coding Sequences in Engineered Host Cells

Engineered host cells made in Example 1 (Strain 102) were transformed with a linearized 3 copy hac1 plasmid (G418R; not shown) and five unmarked egg white protein (EWP or GOI) expression cassettes that possess homology (throughout their 5′ untranslated promoter regions through the 3′ aox1 TT) to five distinct expression constructs that exist in the background of the host cell library (see FIGS. 4A-D):
Coding constructs: 3 copies of hac1-G418R;
FDH1-aMF-GOI-aox1TT
FGH1-aMF-GOI-aox1TT
AOX1-aMF-GOI-aox1TT
shDAS1-aMF-GOI-aox1TT
FLD1-aMF-GOI-aox1TT

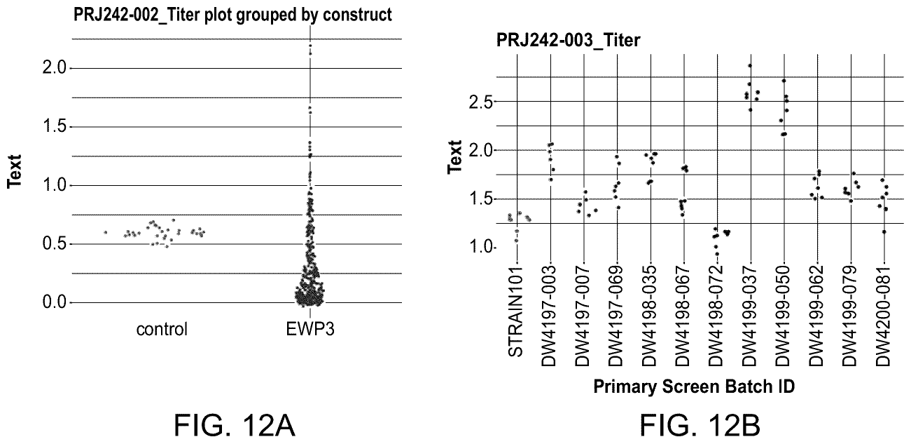

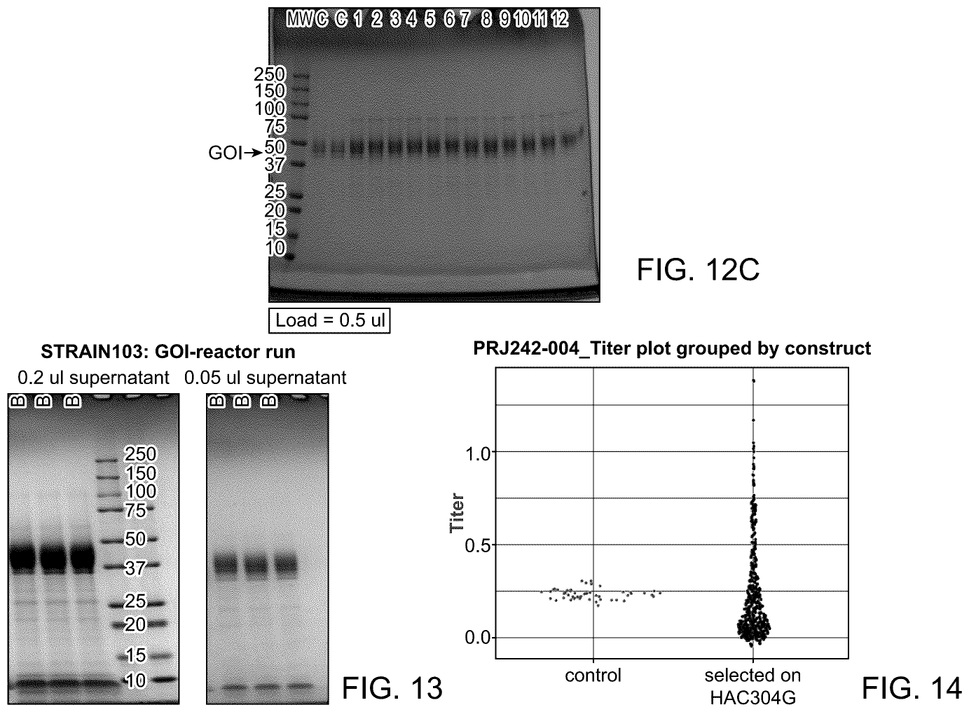

Three hundred twenty primary transformants were screened in 96-deep well plates for EWP (GOI) expression following a 96-hour time course in methanol-containing induction media. The distribution of hits, assayed by protein titer measurement using the Bradford reagent, are displayed in FIG. 12A. Cell culture supernatants were further analyzed by SDS-PAGE and several strains possessing the highest GOI protein titers were subjected to purification by single colony isolation and these strains were then re-screened in a secondary 96 hour time course, their protein titer distributions are shown in FIG. 12B below and the culture supernatants further analyzed by SDS-PAGE, FIG. 12C.

Sequencing the genome of one of the high titer host cells showed that they contained 10 copies of GOI and 3 copies of HAC1. FIGS. 13A-C illustrate the expression construct integrations in chromosome 2. The unique barcode sequences suggest five separate expression constructs guided insertion events (red). Some copies seem to have integrated alongside the guided events (green), possibly during DNA repair via NHEJ.

FIG. 14 shows the SDS-PAGE analysis of a high-titer strain bioreactor run. In the first bioreactor run (duplicate DASGIP 2 L reactors) GOI reached 17.6 g/L (n=2) as measured using the Bradford reagent and a chicken egg white protein standard. From left to right in the left panel are 0.2 ul of DASGIP tank supernatant #15 followed by two duplicate lanes of reactor #16 and two lanes of BioRad pre-stained MW marker. The panel on the right is loaded with 0.05 ul of DASGIP tank supernatant #15 followed by two duplicate lanes of reactor #16.

In this example, the present methods, engineered host cells, and kits provided high expression of an illustrative protein, here characterized by GOI.

### Example 4: Clone-Free Secretion of Another Egg White Protein (GOI)

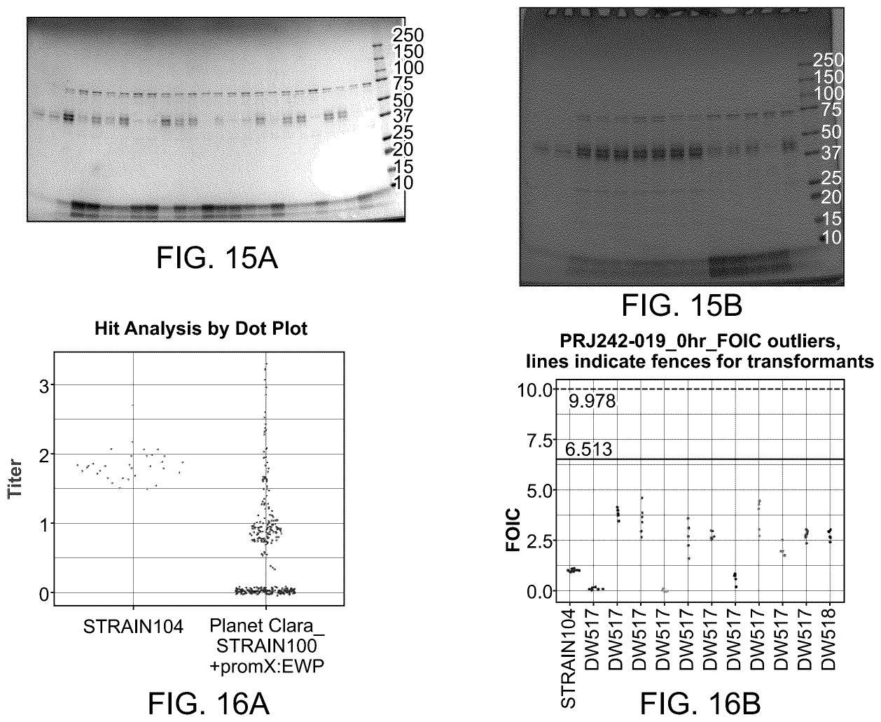

Using PCR combined with the high-fidelity DNA polymerase, Q5, a cDNA encoding αMF-egg white protein 5 (EWP5 or GOI) through the 3′ untranslated aox1 transcriptional terminator (aox1 TT) was amplified, then co-transformed with a linearized selectable marker that encodes three copies of the transcriptional activator HAC1p [wzHAC304G-lox G418 co-transformation] for selection on YPD with 2 mg/ml G418. G418R colonies were isolated into DW plates and subjected to a 7-day time course under methanol induction. Cell-free supernatants were analyzed by Bradford colorimetric assays (FIG. 14. EWP5 Hit Distribution) and SDS-PAGE (FIG. 15A) lanes of which are shown below in Table 7.

TABLE 7
SDS-PAGE Lane assignements
HTS 171 promoterless alphaMF-EWP5

| | HTS Primary | | | | mg/ml | |
|---|---|---|---|---|---|---|
| lane | Plate ID | Coordinate | Notes | | calculated | FOIC* |
| 1 | DW4442 | E5 | STRAIN112 - | | 0.28 | 1.07 |
| | | | positive control | | | |
| 2 | DW4442 | E5 | STRAIN112 | | 0.28 | 1.07 |
| 3 | DW4442 | A11 | Alpha mating | transformant | 1.39 | 5.34 |
| | | | factor- EWP5 | | | |
| 4 | DW4437 | F1 | Alpha mating | transformant | 1.38 | 5.81 |
| | | | factor- EWP5 | | | |
| 5 | DW4439 | F3 | Alpha mating | transformant | 1.17 | 5.17 |
| | | | factor- EWP5 | | | |
| 6 | DW4442 | GB | Alpha mating | transformant | 1.05 | 4.04 |
| | | | factor- EWP5 | | | |
| 7 | DW4439 | E11 | Alpha mating | transformant | 1.03 | 4.55 |
| | | | factor- EWP5 | | | |
| 8 | OW4437 | A3 | Alpha mating | transformant | 1.02 | 4.27 |
| | | | factor- EWP5 | | | |
| 9 | DW4440 | A3 | Alpha mating | transformant | 1.00 | 4.75 |
| | | | factor- EWP5 | | | |
| 10 | DW4442 | H9 | Alpha mating | transformant | 0.97 | 3.72 |
| | | | factor- EWP5 | | | |
| 11 | DW4442 | H10 | Alpha mating | transformant | 0.93 | 3.59 |
| | | | factor- EWP5 | | | |
| 12 | DW4437 | GS | Alpha mating | transformant | 0.92 | 3.87 |
| | | | factor- EWP5 | | | |
| 13 | DW4438 | G3 | Alpha mating | transformant | 0.91 | 3.92 |
| | | | factor- EWP5 | | | |
| 14 | DW4437 | G9 | Alpha mating | transformant | 0.91 | 3.81 |
| | | | factor- EWP5 | | | |
| 15 | DW4441 | A9 | Alpha mating | transformant | 0.88 | 3.81 |
| | | | factor- EWP5 | | | |
| 16 | DW4438 | A11 | Alpha mating | transformant | 0.87 | 3.77 |
| | | | factor- EWP5 | | | |
| 17 | DW4437 | H5 | Alpha mating | transformant | 0.84 | 3.55 |
| | | | factor- EWP5 | | | |
| 18 | DW4440 | B10 | Alpha mating | transformant | 0.84 | 3.97 |
| | | | factor- EWP5 | | | |
| 19 | DW4441 | D5 | Alpha mating | transformant | 0.83 | 3.63 |
| | | | factor- EWP5 | | | |
| 20 | DW4438 | H5 | Alpha mating | transformant | 0.82 | 3.56 |
| | | | factor- EWP5 | | | |
| 21 | DW4439 | H7 | Alpha mating | transformant | 0.82 | 3.62 |
| | | | factor- EWP5 | | | |
| 22 | DW4440 | F10 | Alpha mating | transformant | 0.82 | 3.89 |
| | | | factor- EWP5 | | | |
| 23 | DW4440 | H9 | Alpha mating | transformant | 0.77 | 3.63 |
| | | | factor- EWP5 | | | |
| 24 | DW4437 | B5 | Alpha mating | transformant | 0.76 | 3.18 |
| | | | factor- EWP5 | | | |
| 25 | OW4440 | H8 | Alpha mating | transformant | 0.75 | 3.57 |
| | | | factor- EWP5 | | | |
| 26 | MW Ladder | | | | | |

Following purification by single colony isolation on YPD media containing 2 mg/ml G418, strains expressing GOI were subjected to a secondary time course and re-analyzed for GOI secretion by Bradford analysis and SDS-PAGE (FIG. 15B) lanes of which are described in Table 8.

TABLE 8
SDS-PAGE Lane assignments

| | | HTS Rescreen | | Assay |
|---|---|---|---|---|
| lane | HTS Rescreen | Pia• | Coordinate | Calculated |
| 1 | rDW4465 | rDW4465-053 | E5 | 0.31 |
| 2 | rDW4465 | rDW4465-053 | E5 | 0.31 |
| 3 | rDW4465 | rDW4465-037 | D1 | 1.50 |
| 4 | rDW4465 | rDW4465-073 | G1 | 1.44 |
| 5 | rDW4465 | rDW4465-025 | C1 | 1.42 |
| 6 | rDW4465 | rDW4465-061 | F1 | 1.41 |
| 7 | rDW4465 | rDW4465-085 | H1 | 1.41 |
| 8 | rDW4465 | rDW4465-013 | B1 | 1.40 |
| 9 | rDW4465 | rDW4465-049 | E1 | 1.39 |
| 10 | rDW4465 | rDW4465-050 | E2 | 1.36 |
| 11 | rDW4465 | r0W446S-062 | F2 | 1.31 |
| 12 | rDW4465 | rDW4465-086 | H2 | 1.24 |
| 13 | rDW4465 | rDW4465-038 | D2 | 1.22 |
| 14 | rDW4465 | rDW4465-059 | E11 | 1.21 |
| 15 | marker | | | |

DNA sequencing of the genome of one of the high titer host cells showed that it contains 3 copies of EWP5 and 4 copies of HAC1, all embedded within a single multicopy gene array that spans ˜40 kb.

In this example, the present methods, engineered host cells, and kits provided high expression of an illustrative protein, here characterized by EWP5.

### Example 5: Simultaneous Integration of Multicopy Expression Cassettes with Genes of Interest

This example demonstrates that markerless target gene(s) (the gene(s) of interest (goi)) can be delivered simultaneously with expression cassettes containing an antibiotic resistance gene. In other words, a goi open reading frame (orf) flanked by promoter X and terminator Y sequences can be integrated into promoter X-terminator Y expression cassettes during integration into the host cell genome.

A K. phaffii strain carrying three extra copies of hac1 (Strain 100 methanol utilization slow MutS) was transformed with a PCR product of an inducible promoter-goi (egg white protein or EWP)-AOX1 terminator as the goi (unmarked) alongside (piggybacked) a vector carrying four copies of the inducible promoter-AOX1 transcriptional terminator (TT) expression cassette (LP) and a Zeocin antibiotic resistance marker. Transformants were selected on YPD plus zeocin plates (500 ug/ml) and then subjected to a timecourse analysis to identify high producing GOI strains (see FIG. 16A-B).

FIG. 16A illustrates the total distribution of transformants (dots on the right) is compared to control wells (dots on the left) containing the previous best of EWP strain 104. FIG. 16B illustrates the fold over internal control (FOIC) values presented with some strains exhibiting over 2-fold better titers than strain 104 represented with red dots on the left (see FIG. 16B).

High producing strains were further subjected to a PCR diagnostic test that identified only those inducible promoter-EWP-AOX1-TT copies that are integrated into the inducible promoter-AOX1 LP's.

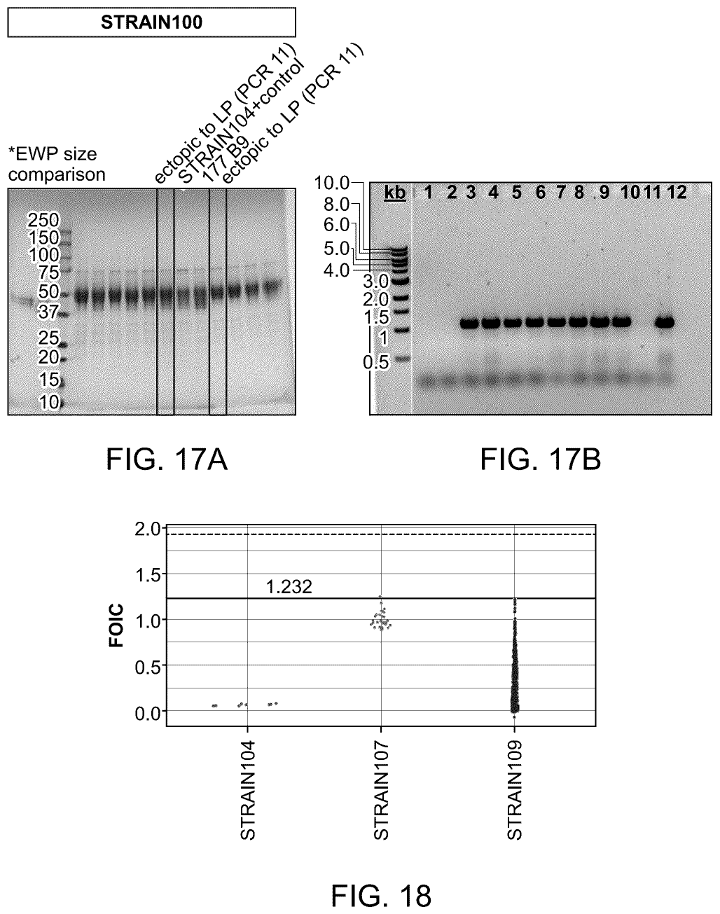

FIG. 17A illustrates results of a diagnostic PCR for LP Integration. In the agarose gel shown in FIG. 17A, twelve separate PCR reactions (Lanes numbered 1-12) corresponding to individual transformants were loaded to diagnose whether or not the goi is on the intended LP. A positive reaction, or a band that migrates at 891 bp is diagnostic for promoter-EWP integrated into an inducible pro-AOX1tt LP (a molecular weight ladder was loaded in the first lane, left). These results suggest that the goi preferentially integrates into homologous LP's (9/12 positive for the got on the intended LP).

FIG. 17B illustrates the SDS-PAGE Analysis of EWP expressing transformants.

In the SDS-PAGE gel of FIG. 17A, supernatants from several different titer ranges of EWP transformants were analyzed. The positive control strain 104 was loaded in the center of the gel (as indicated) and was flanked by wells containing supernatant from a strain where the goi is ectopic to the LP (at left in box) and one where the goi is integrated into the LP (well 177 B9). The gel contains one other example of a goi that is ectopic to the LP (box with “PCR 1” label). Two forms of recombinant egg white protein (EWP) were loaded into the first two left lanes at left for molecular weight reference.

In this example, the present methods, engineered host cells, and kits provided high expression of an illustrative protein, here again characterized by EWP.

### Example 6: Perpetual Marker Removal

In an effort to reserve antibiotic resistance markers during goi delivery, a new LP library was constructed in a Δura3 deletion strain background that also contains the methanol-utilization slow mutation (Δaox2; MuiS). For this library construction a plasmid carrying three additional copies of hac1 were also employed and it contained the same Komagataella pastoris ura3+ (KPASura3) gene as the LP's for selection of prototrophs. Complementation of uracil auxotrophy using cross-species genetic complementation may result in duplication events as the K. pastoris ura3 gene is not as efficient as the K. phaffii ura3 gene at restoring uracil prototrophy (data not shown). For this library construction the following multicopy LP plasmids were employed: “wz304-KPASura3-NORS” (3×hac1 driven by the following promoters; FLD1/DAK2/PEX11, followed by KPAS ura3 and a nourseothricin selectable marker (NORS) contained between direct lox repeats), “4×_uniq3-KPASura3_lox-NORS”, “4×_uniq1uniq3(×2)-KPASura3_lox-NORS”, “6×_uniq2(×4)uniq5(×2)-KPASura3_lox-NORS”, 6×_uniq1(×2)uniq2(×2)uniq3(×2)-KPASura3_lox-NORS, 6×_uniq1(×2)uniq2(×2)uniq5(×2)-KPASura3_lox-NORS.

This library was selected for its ability to grow out in media minus uracil followed immediately by competent cell preparation. A competent library aliquot was then transformed with unmarked expression cassettes corresponding to AOXpro-EWP-AOX1tt, DAS1pro-EWP-AOX1tt, and FDH1pro-EWP-AOX1tt all piggybacked behind a vector backbone “clox-HYGROMYCIN” that contains cre recombinase between direct lox sites and the selectable marker for hygromycin resistance. This system allowed for removal of DNA elements contained between direct lox repeats, ultimately resulting in strains that are completely devoid of all antibiotic resistance markers (ARM-free), by the end of the initial screen.

### Example 7: Iterative Builds and Combinatorial Helper Factor Screening

Strain 108 is a productive egg white protein expressor that has a titer of ˜3.4 mg/ml in deep 96-well plates. As this strain is originally ARM-free (see Example 6) it was transformed with a construct that contains a 6× multicopy LP for genes flanked by the TKL3 promoter and the MOX terminator (6×_uniq7_loxZEO); the selectable marker was a Zeocin resistance gene and therefore transformants were pooled as a library and selected for their ability to grow out in liquid media containing 500 ug/ml Zeocin and then immediately subjected to a competent cell prep. This new library, built in the background of strain 108 was then subjected to a similar piggyback transformation (see Example 6) with the “clox-HYG” vector backbone however, this time the unmarked expression cassettes (TKL3pro-MDH1-MOXtt and TKL3pro-ADK1-MOXtt) encode helper factor genes flanked by the TKL3 promoter and the MOX terminator.

### Example 8: New Elements

Several known promoters are strongly derepressed upon glucose exhaustion, independent of inducers (e.g., methanol) and additionally, some promoters appear to be transcribed during the later timepoints of bioreactor fermentation runs. LP's were designed around a few of these elements in an effort to capture their transcriptional activities and direct them towards a balanced goi expression. These new elements included promoters for methanol independent promoters, late response promoters and de-repression promoters. All expression cassettes had unique extension sequences which act as recognition sequences facilitating homology or detection of the cassettes when integrated in the host genome. The expression cassettes were designed to contain: Barcode (uniqX)-Promoter (pro)-Terminator (tt).

Library Construction: Four multicopy landing pad constructs were constructed in order to build strains that express goi's without the need for methanol as a carbon source (in certain procedures, a small amount of methanol may be included as an inducer molecule). For each promoter-terminator LP combination, a four-copy construct (4×) was built, and all possessed the Zeocin resistance gene contained within direct lox sites. Following DNA construction, 10 ug of each linearized multicopy plasmid was mixed together and electroporated into a K. phaffii strain (strain 105) that is methanol-use reduced and further possessed six additional copies of hac1 under various methanol-independent promoters.

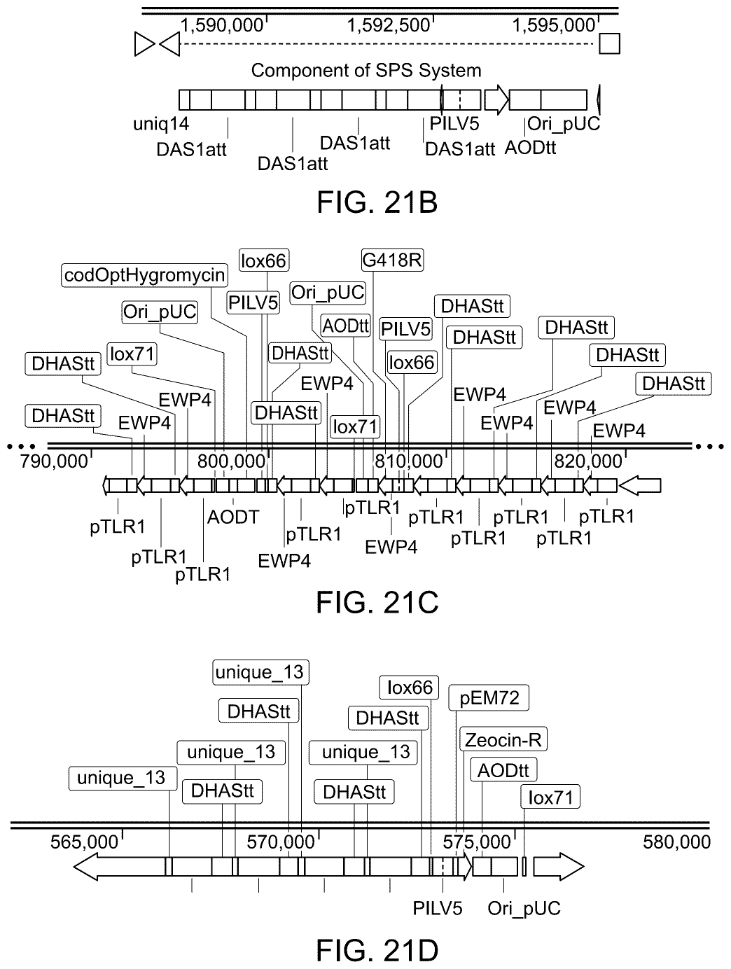

Following library construction, three unmarked expression cassettes representing EWP1 gene cloned behind metabolite de-repression promoters were piggybacked behind the clox-HYG backbone by electroporation into this library (as described above). Individual transformants were picked into deep-well plates and subjected to timecourse expression analysis in various media. The results of these preliminary tests are shown in FIG. 21.

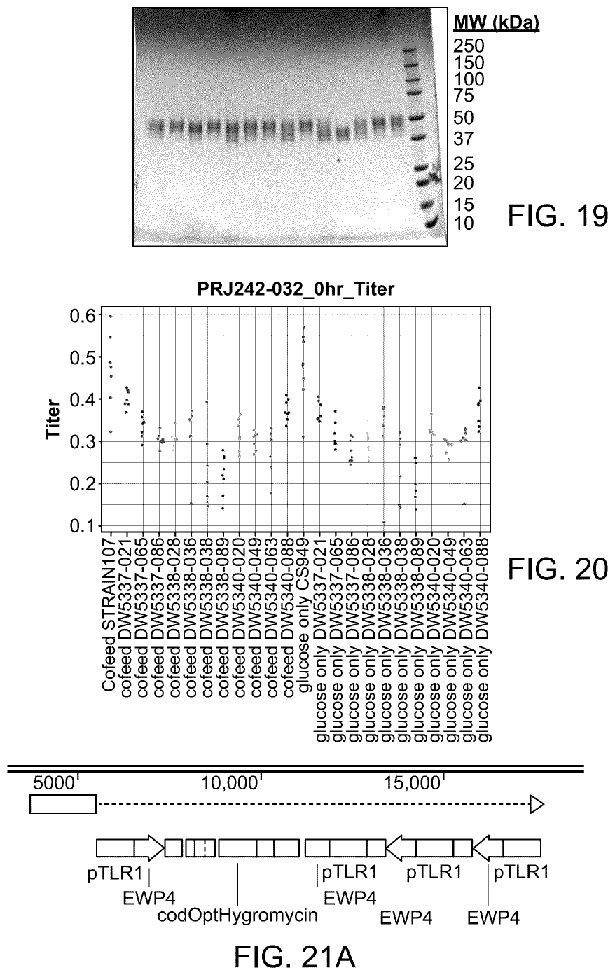

In FIGS. 19 and 20, an example is presented where transformants expressing an egg white protein (GOI) from methanol-independent promoters are taken through timecourse expression studies in the presence or absence of methanol. In the first timecourse, transformants undergo a low methanol feeding schedule where once every 24 h they are given a bolus of methanol and glucose (0.1/1% final conc. respectively). This was performed to induce cre expression on the clox-HYG backbone resulting in ARM removal. Following this primary screen, some transformants that performed well in the primary screen were selected to undergo a re-screen where single colony isolates were subjected to the identical low methanol feed schedule in addition to a complete media switch to glucose only, the titers of several transformants are shown in FIG. 20 “0 hr” prior to their media switch to the low methanol feeding schedule or “glucose only”.

In FIG. 18, egg white protein (GOI)-expressing transformants (strain 109; blue dots on the right) had been under a low methanol feeding regimen for 96 hrs. as the two control strains (strain 104 in red dots on the left; Strain 107 in pink dots) also presented. Strain 104 expresses GOI from methanol-dependent promoters and does not express a high amount of protein under the low methanol feed schedule.

FIG. 19: In each lane above 1 μl of cell-free supernatant was loaded from the top twelve transformants expressing GOI from methanol-independent promoters following a 96 h timecourse. The lane assignments are given in Table 9 below.

TABLE 9
Lane assignments
HTS-210 oOVA Me OH-free

| | | | Assay | |
|---|---|---|---|---|
| Lane | HTS Primary Plate 10 | Notes | Calculated | FOIC* |
| 1 | DW5340 | | 1.3075 | 1.2139 |
| 2 | DW5340 | | 1.2740 | 1.1828 |
| 3 | DW5337 | | 1.2431 | 1.1568 |
| 4 | DW5338 | | 1.2229 | 1.2507 |
| 5 | DW5338 | | 1.2159 | 1.2435 |
| 6 | DW5337 | | 1.1970 | 1.1139 |
| 7 | DW5338 | | 1.0969 | 1.1218 |
| 8 | DW5338 | | 1.0957 | 1.1206 |
| 9 | DW5340 | | 1.0815 | 1.0040 |
| 10 | DW5337 | | 1.0397 | 0.9675 |
| 11 | DW5340 | | 1.0255 | 0.9521 |
| 12 | DW5338 | | 1.0211 | 1.0443 |
| 13 | Positive control | Strain 107 | 1.2879 | 1.2478 |
| 14 | Positive control | | 1.2678 | 1.1770 |
| 15 | MWLadder | | | |

FOIC*—Fold Over Internal Control

Following this primary timecourse, transformants were subjected to a re-screen under identical conditions and also in a completely methanol-free media (described above). The “0 hr” protein titers of these selected transformants are shown in the FIG. 20 “0 hr Expression” and this represents the amount of protein they express in the 72 hrs prior to the media switch as discussed above. Several of these “Methanol-free” transformants are expressing the GOI at titers near the level of Strain 107 however they are different, in that the same promoter-GOI was not used for this strain building example.

### Example 9: Highly Productive Strains for Ovomucoid

Strain 106: This is a highly productive EWP4-expressing strain in which multicopy (MC) expression cassettes (LP's) were installed for further copy number and physiological improvements.

Initially a library was created in strain 106 using 2 different versions of 4× late firing promoter (pro)-Das1 terminator (aTT) named strain 113.

The strain 113 library was now usable for any type of genetic improvement or interrogation with genes flanked by the same 5′ and 3′ homologies of the two 4×MC LP's mentioned above (5′ region contains late firing promoters, and the 3′ region contains the transcriptional terminator Das1aTT).

Additional copies of late firing promoter (TLR1)-EWP4-Das1aTT were transformed into strain 113 and high level EWP4 expressing strains were selected for in a small screen (384-well experiment) where strain 106 was used as a positive control.

Two improved strains, strain 110 and strain III performed 16% and 23% (respectively) better than the parent strain 106 in the HTS screen and were subjected to further evaluation in bioreactors.

The genetics and protein expression performances of strain 110 (FIG. 21A-B) and strain 111 (FIG. 21 C-D) are diagrammed in FIGS. 21A-D.

Strain 110 contains: 4×TLR1-EWP4 inserted into chromosome 1. One copy of EWP4 was found to be truncated and not flanked by a DAStt. This cassette contains a hygromycin ARM and disrupts a gene for a cytosolic protein required for sporulation.

Strain 110 contains: 4× empty TLR1-DAS1att expression cassettes inserted into chromosome 1. This cassette contained a zeocin ARM and disrupts a component of the SPS amino acid sensing system and signal transduction pathway.

Strain 111 contains: 9×TLR1-EWP4 cassettes inserted into chromosome 4. This insert contains a hygromycin and G418R ARM.

Strain 111 contains: 4× empty expression cassettes inserted into chromosome 2. This insert contains a zeocin ARM.

Both of the new strains possess the desired genetic outcomes (additional EWP4 copies) and both contain additional, empty LP's that could accept even more targeted genes

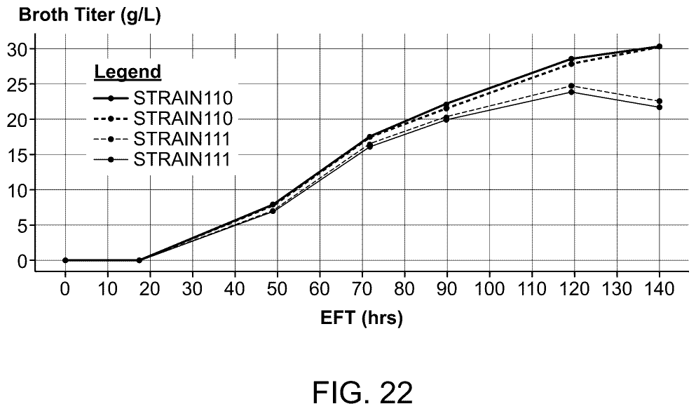

When broth titers of the new strains are compared (see FIG. 22), Strain 110 performs much better than strain 111 and delivers functional EWP4 protein. Strain 111 plateaus fairly rapidly (see FIG. 22), and some cellular damage was apparent by SDS-PAGE (not shown). Overall, the results suggest that four additional copies of TLR1-EWP4-Das1aTT is beneficial to titer whereas, nine additional copies of TLR1-EWP4-Das1aTT was not as efficient and may have led to issues in the host cell.

Strain 110 performed better (5.8% better broth titer) than the parental strain 106 using a standard fermentation process.

Strain 110 showed an average broth titer of 30.19 g/L EWP4, while strain 111 showed an average broth titer of 22.17 g/L and was unsuccessful.

Two bioreactor runs for each of the strains are shown in timecourse/performance analysis line graph FIG. 22. Strain 110 tanks are red lines and the strain 111 tanks are gray lines.

In this example, the present methods, engineered host cells, and kits provided high expression of an illustrative protein, here again characterized by EWP4.

### Example 10: Strains for Non-Food Proteins

Engineered host cells made in Example 1 (Strain 102) can be transformed with multiple copies of any type of proteins such as a protein suitable as a biopharmaceutical substance like an antibody or antibody fragment, growth factor, hormone, enzyme, vaccine, regulatory protein, receptor, cytokine, antigen-binding proteins, immune stimulatory proteins, scaffold binding protein, structural protein, lymphokine, adhesion molecule, membrane or transport protein, other polypeptides that can serve as an agonist or antagonist and/or have therapeutic or diagnostic use, or a protein which can be used for industrial or cosmetic application. The coding constructs can be designed to possess homology (throughout their 5′ untranslated promoter regions through the 3′ terminators) to various distinct expression constructs that exist in the background of the host cell library.

In some cases, a strain made using Example 1 can have any combination of promoters such as listed in Table 1, terminators such as listed in Table 2 and signal peptides (optionally) such as listed in Table 3.

The host cell can also be selected from any host organisms described herein.

Primary transformants can be screened as is described in Example 3 or other examples described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. An engineered host cell for expressing one or more heterologous genes, the engineered host cell comprising a plurality of expression cassettes integrated into the genome of the engineered host cell, the engineered host cell comprising:
a. the plurality of expression cassettes each having two or more transcriptional elements,
wherein at least one of the expression cassettes comprises a combination of a set of transcriptional elements that are non-native to the engineered host cell, and
b. each of the plurality of expression cassettes lacks a sequence of the one or more heterologous genes,
wherein the engineered host cell is capable of integrating a plurality of coding constructs into the expression cassette without requiring a nuclease enzyme,
wherein each coding construct comprises the sequence of at least one of the heterologous genes and at least a sequence homologous to the expression cassette or a partial sequence thereof.

2. The engineered host cell of claim 1, wherein the plurality of expression cassettes comprises at least two different expression cassettes integrated into the genome of the engineered host cell.

3. The engineered host cell of claim 1, wherein each of the plurality of expression cassettes does not comprise a nuclease targeting sequence.

4. The engineered host cell of claim 1, wherein the transcriptional elements non-native to the engineered host cell are selected from the group consisting of a promoter, a terminator sequence, a signal sequence, or combinations thereof.

5. The engineered host cell of claim 1, wherein one or more of the plurality of expression cassettes comprise an inducible promoter, a regulated promoter, a repressible promoter, and/or a constitutive promoter.

6. The engineered host cell of claim 1, wherein one or more of the plurality of coding constructs lacks a full-length promoter sequence, an operable promoter sequence, or a promoter sequence native to the engineered host cell.

7. The engineered host cell of claim 1, wherein at least one of the plurality of expression cassettes further comprises a unique barcode sequence.

8. The engineered host cell claim 1, wherein the engineered host cell is a yeast cell.

9. The engineered host cell of claim 8, wherein two or more of the plurality of expression cassettes are integrated in different chromosomes in the engineered host cell's genome.

10. The engineered host cell of claim 8, wherein two or more of the plurality of expression cassettes are integrated in the same chromosome in the engineered host cell's genome.

11. The engineered host cell of claim 9, wherein two or more expression cassettes integrated in the same chromosome are oriented in opposite transcriptional directions.

12. The engineered host cell of claim 9, wherein two or more expression cassettes integrated in the same chromosome are oriented in the same transcriptional direction.

13. The engineered host cell of claim 1, wherein the engineered host cell is a bacterial cell.

14. The engineered host cell of claim 1, wherein at least two of the plurality of expression cassettes comprise different promoters, different secretion signal sequences, different terminator sequences, or combinations thereof.

15. The engineered host cell of claim 1, wherein the engineered host cell comprises one or more integrated helper expression cassettes comprising a promoter driving expression of a helper protein.

16. A method of expressing one or more heterologous genes in an engineered host cell, the method comprising:
a. introducing a plurality of coding constructs into the host cell,
wherein the host cell comprises a genome having a plurality of integrated expression cassettes each lacking a sequence of the one or more heterologous genes,
wherein each coding construct comprises a sequence of at least one of the one or more heterologous genes and a first 5′ recognition zone comprising at least a sequence homologous to the expression cassette or to a partial sequence thereof; and
b. incubating the engineered host cell and the plurality of coding constructs in conditions that allow homologous recombination of the one or more coding constructs comprising the sequence of one or more heterologous genes with the expression cassettes, thereby integrating the sequence of one or more heterologous genes into the engineered host cell genome;
wherein at least one of the plurality of expression cassettes comprises two or more transcriptional elements that are non-native to the engineered host cell;
wherein the engineered host cell is capable of integrating the sequence of the one or more heterologous genes into the expression cassette without requiring a nuclease enzyme.

17. The method of claim 16, wherein at least one of the plurality of coding constructs is vector-less.

18. The method of claim 16, wherein each of the plurality of expression cassettes does not comprise a nuclease targeting sequence.

19. The method of claim 16, wherein at least one of the plurality of coding constructs does not comprise an origin of replication for a plasmid or vector.

20. The method of claim 16, wherein at least one of the plurality of coding constructs is a linear DNA fragment.

21. The method of claim 16, wherein at least one of the plurality of coding constructs lacks regulatory elements operably linked to the coding sequence of the heterologous gene.

22. The method of claim 16, wherein at least one of the plurality of coding constructs lacks a full-length promoter sequence, an operable promoter sequence, or a promoter sequence native to the engineered host cell.

23. The method of claim 16, wherein the first 5′ recognition zone comprises at least 50 nucleotides located 5′ to the coding sequence of the heterologous gene.

24. The method of claim 23, wherein the sequence of the 5′ recognition zone is homologous to a portion of the promoter sequence or a signal peptide sequence in one or more of the plurality of expression cassettes.

25. The method of claim 16, wherein at least one or each of the plurality of coding constructs comprises a first 3′ recognition zone comprising at least 50 nucleotides located 3′ to the coding sequence of the heterologous gene.

26. The method of claim 25, wherein the sequence of the 3′ recognition zone is homologous to portion of a terminator sequence in one or more of the plurality of expression cassettes.

27. The method of claim 16, wherein at least two different coding constructs are transformed into the engineered host cell.

28. The method of claim 27, wherein the two different coding constructs comprise the coding sequence for the same heterologous gene but comprise a different 5′ or 3′ recognition zone flanking the coding sequence of the heterologous gene.

29. The method of claim 16, wherein the introduction comprises transformation and the coding constructs are transformed into the engineered host cell simultaneously.

30. The method of claim 16, wherein at least 3, 4, 5, 6, 7, 8, 9 or 10 different coding constructs are transformed into the engineered host cell simultaneously.

31. The method of claim 16, wherein each coding construct comprises the coding sequence of the same heterologous gene.

32. The method of claim 16, wherein the plurality of the coding constructs comprises the coding sequence of at least two different heterologous genes.

33. The method of claim 16, wherein the transcriptional elements non-native to the engineered host cells are selected from the group consisting of a promoter, a terminator sequence, a signal sequence non-native to the host cell, or combinations thereof.

34. The method of claim 33, wherein at least one of the combinations of transcriptional elements comprises a promoter sequence non-native to the host cell.

35. The method of claim 16, wherein one or more of the plurality of expression cassettes comprise an inducible promoter, a regulated promoter, a repressible promoter, and/or a constitutive promoter.

36. The method of claim 16, wherein at least one promoter in the plurality of expression cassettes comprises a unique barcode sequence.

37. The method of claim 16, wherein the engineered host cell is a yeast cell.

38. The method of claim 16, wherein two or more of the plurality of expression cassettes are integrated in different chromosomes in the engineered host cell's genome.

39. The method of claim 16, wherein two or more of the plurality of expression cassettes are integrated in the same chromosome in the engineered host cell's genome.

40. The method of claim 38, wherein two or more expression cassettes integrated in the same chromosome are oriented in opposite transcriptional directions.

41. The method of claim 38, wherein two or more expression cassettes integrated in the same chromosome are oriented in the same transcriptional direction.

42. The method of claim 16, wherein the engineered host cell is a bacterial cell.

43. The method of claim 16, wherein at least two of the plurality of expression cassettes comprise different promoters, different secretion signal sequences, different terminator sequences, or combinations thereof.

44. The method of claim 16, wherein the engineered host cell comprises one or more integrated helper expression cassettes comprising a promoter driving expression of a helper protein.

45. The method of claim 44, wherein the method further comprises removing one or more expression cassettes that do not comprise the coding construct from the engineered host cell after step (b).

46. The method of claim 45, wherein the removing of the one or more expression cassettes is performed by inducing a double stranded break in or near the expression cassette.

47. The method of claim 46, wherein the double stranded break is not induced by a Cas enzyme.

48. The method of claim 16, wherein the method further comprises culturing the engineered host cell in fermentation media and measuring an amount of the protein expressed by the one or more heterologous genes.

49. The method of claim 16, wherein the method further comprises incubating the engineered host cell in conditions that allow integration of a second plurality of expression cassettes into the engineered host genome;
wherein each of the second plurality of expression cassettes comprises two or more transcriptional elements selected from the group consisting of a promoter, signal sequence and terminator sequence; and
wherein at least one of the expression cassettes in the second plurality comprises a set of transcriptional elements that are non-native to the engineered host cell.

50. The method of claim 49, wherein the method further comprises
transforming a second plurality of coding constructs into the engineered host cell, each construct comprising a coding sequence for a heterologous gene;
incubating the engineered host cell with the second plurality of coding constructs in conditions that allow homologous recombination of the second plurality of coding constructs with the engineered host cell, thereby integrating the second plurality of coding constructs into the engineered host cell.

51. The method of claim 16, wherein the method further comprises sequencing the engineered host cell genome.

52. A method of producing an engineered host cell comprising,
a. transforming a plurality of expression cassettes into the engineered host cell, the plurality of expression cassettes each lacking a coding sequence of a heterologous gene to the engineered host cell;
b. incubating the engineered host cell in conditions that allow integration of the plurality of different expression cassettes into the host genome;
wherein at least one of the plurality of expression cassettes comprises two or more transcriptional elements non-native to the engineered host cell,
wherein the transcriptional elements are selected from the group consisting of a promoter, signal sequence, and terminator sequence; and
wherein the engineered host cell is capable of integrating the coding sequence of the heterologous gene into the one or more expression cassettes without requiring a nuclease enzyme.

53. The method of claim 52, wherein each of the plurality of expression cassettes do not comprise a nuclease targeting sequence.

54. The method of claim 53, wherein the engineered host cell comprises at least one heterologous expression cassette capable of driving expression of a heterologous gene sequence in the engineered host cell.

55. The method of claim 54, wherein the engineered host cell comprises at least one heterologous expression cassette driving expression of a helper factor gene sequence.

56. The method of claim 52, wherein the method further comprises mating the engineered host cell with a second host cell.

57. The method of claim 56, wherein the second host cell comprises a plurality of different expression cassettes driving expression of a heterologous gene sequence to the second host cell.

58. The method of claim 57, wherein the second host cell has an antibiotic resistance marker different from an antibiotic resistance marker in the engineered host cell.

59. A kit comprising a plurality of engineered host cells of claim 1 and a set of instructions for culturing the engineered host cells, at least, for expressing recombinant proteins.

60. A library of vectors for transformation of a host cell, wherein the library of vectors comprises a plurality of expression cassettes wherein:
a. at least one of the plurality of expression cassettes comprises two or more transcriptional elements non-native to the engineered host cell,
a. wherein the transcriptional elements are selected from the group consisting of a promoter and a terminator sequence, and signal sequence;
b. at least one of the expression cassettes comprises a combination of transcriptional elements that is non-native to the host cell;
wherein one or more of the plurality of expression cassettes lacks a coding sequence of a protein heterologous to the host cell.

61. A library of more than one different engineered host cell lines,
wherein a cell of each of the more than one different engineered host cell lines comprises a plurality of expression cassettes,
wherein at least one of the plurality of expression cassettes comprises two or more transcriptional elements non-native to the engineered host cell,
c. wherein the transcriptional elements are selected from the group consisting of a promoter and a terminator sequence, and signal sequence;
wherein one or more of the plurality of expression cassettes lacks a coding sequence of a protein heterologous to the host cell;
wherein each one of the different engineered host cell lines in the library comprises a different combination of expression cassettes.
